(19) United States
(12) Patent Application Publication
(10) Pub. No.: US 2007/0203147 A1
(43) Pub. Date: Aug. 30, 2007

(54) 2-Aminothiazole Compounds Useful As Aspartyl Protease Inhibitors

(76) Inventors: Craig A. Coburn, Royersford, PA (US); Amy S. Espeseth, Chalfont, PA (US); Shawn J. Stachel, Perkasie, PA (US); David B. Olsen, Lansdale, PA (US); Daria J. Hazuda, Doylestown, PA (US); M. Katharine Holloway, Lansdale, PA (US)

Correspondence Address: MERCK AND CO., INC, P O BOX 2000, RAHWAY, NJ, 07065-0907, US

(21) Appl. No.: 10/593,808

(22) PCT Filed: Mar. 25, 2005
(86) PCT No.: PCT/US05/10224

§ 371 Date: Sep. 20, 2006

Related U.S. Application Data
(60) Provisional application No. 60/557,769, filed on Mar. 30, 2004.
(60) Provisional application No. 60/591,386, filed on Jul. 27, 2004.

(51) Int. Cl. A61K 31/506; A61K 31/501; A61K 31/497; A61K 31/4709; A61K 31/4439; A61K 31/427; A61K 31/426; C07D 417/02

(52) U.S. Cl. 514/252.05; 514/255.05; 514/256; 514/307; 514/314; 514/342; 514/370; 544/238; 544/405; 544/333; 546/148; 546/176; 546/269.7; 548/190

(57) ABSTRACT

The present invention is directed to 2-aminothiazole compounds which are aspartyl protease inhibitors, and are inhibitors of both the beta-secretase enzyme and HIV protease, and that are useful in the treatment of diseases in which the beta-secretase enzyme and HIV are involved, such as Alzheimer's disease, HIV Infection and AIDS. The invention is also directed to pharmaceutical compositions comprising these compounds and the use of these compounds and compositions in the treatment of such diseases in which the beta-secretase enzyme and HIV protease are involved.

### CROSS-REFERENCE TO RELATED APPLICATIONS

This application claims priority under 35 U.S.C. § 119(e) of U.S. provisional application Ser. No. 60/557,769, filed Mar. 30, 2004, and U.S. provisional application Ser. No. 60/591,386, filed Jul. 27, 2004.

### FIELD OF THE INVENTION

The present invention is directed to 2-aminothiazole compounds which are useful as aspartyl protease inhibitors, their pharmaceutically acceptable salts, and their use as inhibitors of the beta secretase protease and HIV protease. The compounds of the present invention are useful for treating Alzheimer's Disease, for treating infection by HIV, and for treating AIDS.

### BACKGROUND OF THE INVENTION

Proteases, or proteolytic enzymes, are common biological control agents present in blood plasma, sperm and various mammalian tissues. Some proteases, such as the aspartyl proteases beta secretase protease and the HIV protease, contribute to the pathophysiology of human diseases. For example, beta secretase causes the production of the amyloid β (Aβ) protein in the brain, which is characteristic of Alzheimer's Disease. Also, the HIV protease is a viral enzyme which is present in the HIV genome, and is necessary for the replication of HIV (Kohl et al., Proc. Nat'l Acadl. Sci. 1988, 85:4686).

The compounds of the invention are useful as inhibitors of both beta secretase and HIV protease, and thus are useful in the treatement of diseases in which beta secretase and HIV protease are involved, such as Alzheimer's Disease, HIV infection and AIDS.

Alzheimer's disease is characterized by the abnormal deposition of amyloid in the brain in the form of extra-cellular plaques and intra-cellular neurofibrillary tangles. The rate of amyloid accumulation is a combination of the rates of formation, aggregation and egress from the brain. It is generally accepted that the main constituent of amyloid plaques is the 4 kD amyloid protein (βA4, also referred to as Aβ, β-protein and βAP) which is a proteolytic product of a precursor protein of much larger size. The amyloid precursor protein (APP or AβPP) has a receptor-like structure with a large ectodomain, a membrane spanning region and a short cytoplasmic tail. The Aβ domain encompasses parts of both extra-cellular and transmembrane domains of APP, thus its release implies the existence of two distinct proteolytic events to generate its NH₂— and COOH-termini. At least two secretory mechanisms exist which release APP from the membrane and generate soluble, COOH-truncated forms of APP (APPₛ). Proteases that release APP and its fragments from the membrane are termed “secretases.” Most APPₛ is released by a putative α-secretase which cleaves within the Aβ protein to release α-APP, and precludes the release of intact Aβ. A minor portion of APPₛ is released by a β-secretase (“β-secretase”), which cleaves near the NH₂-terminus of APP and produces COOH-terminal fragments (CTFs) which contain the whole Aβ domain.

Thus, the activity of β-secretase or β-site amyloid precursor protein-cleaving enzyme (“BACE”) leads to the abnormal cleavage of APP, production of Aβ, and accumulation of β amyloid plaques in the brain, which is characteristic of Alzheimer's disease (see R. N. Rosenberg, Arch. Neurol., vol. 59, September 2002, pp. 1367-1368; H. Fukumoto et al, Arch. Neurol., vol. 59, September 2002, pp. 1381-1389; J. T. Huse et al, J. Biol. Chem., vol 277, No. 18, issue of May 3, 2002, pp. 16278-16284; K. C. Chen and W. J. Howe, Biochem. Biophys. Res. Comm, vol. 292, pp 702-708, 2002). Therefore, therapeutic agents that can inhibit β-secretase or BACE may be useful for the treatment of Alzheimer's disease.

The compounds of the present invention are also inhibitors of HIV protease, and thus are useful for treating HIV infection and AIDS.

HIV is the etiological agent of the complex disease that includes progressive destruction of the immune system (acquired immune deficiency syndrome; AIDS) and degeneration of the central and peripheral nervous system. A common feature of retrovirus replication is the extensive post-translational processing of precursor polyproteins by a virally encoded protease to generate mature viral proteins required for virus assembly and function. Inhibition of this processing prevents the production of normally infectious virus. For example, Kohl et al., Proc. Nat'l Acad. Sci. 1988, 85: 4686, demonstrated that genetic inactivation of the FHV encoded protease resulted in the production of immature, non-infectious virus particles. These results indicated that inhibition of the HIV protease represents a viable method for the treatment of AIDS and the prevention or treatment of infection by HIV.

Nucleotide sequencing of HIV shows the presence of a pol gene in one open reading frame [Ratner et al., Nature 1985, 313: 277]. Amino acid sequence homology provides evidence that the pol sequence encodes reverse transcriptase, an endonuclease and an HIV protease [Toh et al., EMBO J. 1985, 4: 1267; Power et al., Science 1986, 231: 1567; Pearl et al., Nature 1987, 329: 351].

Several HIV protease inhibitors are presently in clinical use for the treatment of AIDS and HIV infection, including indinavir (see U.S. Pat. No. 5,413,999), nelfinavir (U.S. Pat. No 5,484,926), saquinavir (U.S. Pat. No. 5,196,438), and ritonavir (U.S. Pat. No. 5,484,801). Each of these protease inhibitors is a peptidomimetic, competitive inhibitor of the viral protease which prevents cleavage of the HIV gag-pol polyprotein precursor.

### SUMMARY OF THE INVENTION

The present invention is directed to 2-aminothiazole compounds useful as inhibitors of the β-secretase enzyme, and as inhibitors of HIV protease. The invention is also directed to pharmaceutical compositions comprising these compounds, and the use of these compounds and compositions in the treatment of such diseases in which the β-secretase enzyme and HIV protease is involved.

### DETAILED DESCRIPTION OF THE INVENTION

The present invention is directed to compounds of formula (I): 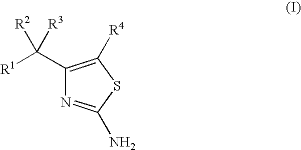
wherein:
R¹ is selected from the group consisting of:

(1) —C1-6alkyl,

(2) —C2-6 alkenyl,

(3) —C₀₋₆alkyl-C₃₋₆ cycloalkyl,

(4)

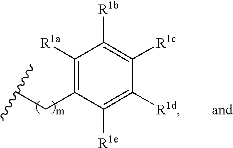

(5) heteroaryl selected from the group consisting of furyl, pyranyl, benzofuranyl, isobenzofuranyl, chromenyl, thienyl, benzothiophenyl, pyrrolyl, pyrazolyl, imidazolyl, pyridyl, pyrazinyl, pyrimidinyl, pyridazinyl, indolyl, indazolyl, benzimidazolyl, quinolyl and isoquinolyl,
wherein
(a) said alkyl, alkenyl or cycloalkyl is unsubstituted or substituted with one or more halogen, —C₁₋₆alkyl, —C₁₋₆alkoxy, hydroxy or cyano, and
(b) said heteroaryl is unsubstituted or substituted with one or more halogen, —C₁₋₆alkyl, —C₁₋₆alkoxy, phenyl, hydroxy or cyano,

and wherein R^{1a}, R^{1b}, R^{1c}, R^{1d} and R^{1e} are selected from the group consisting of:
(a) hydrogen,
(b) halogen,
(c) cyano,
(d) hydroxyl,
(e) —C₁₋₆ alkoxy,
(f) —C(═O)—O—R^{7a},
(g) —O—CO₁₋₆alkyl-C(═O)—R^{7a},
(h) —N—R^{7a}—S(O)ₚ—R^{7b},
or R^{1b} and R^{1c} are linked together to form —O—CH₂—O— or —CH═CH—CH═CH—;
wherein said aryl is unsubstituted or substituted with one or more halogen, —C₁₋₆alkyl, —C₁₋₆alkoxy, hydroxyl or cyano;
R² is selected from the group consisting of:

(1) hydrogen,

(2) halogen,

(3) —C₀₋₆alkyl-Q¹-C₁₋₆alkyl, wherein Q¹ is O or S,

(4) —C₁₋₆alkyl, and

(5) hydroxyl;
R³ is selected from the group consisting of:

(1) hydrogen,

(2) —C₁₋₆alkyl,

(3) —C₀₋₆alkyl-C₃₋₆cycloalkyl,

(4) —C₀₋₆alkyl-Q²-C₁₋₆alkyl, wherein Q² is O, S or —C(═O)—O—, and

(5)

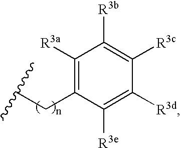

(6) —CH₂-heteroaryl, wherein said heteroaryl is selected from the group consisting of furyl, pyranyl, benzofuranyl, isobenzofuranyl, chromenyl, thienyl, benzothiophenyl, pyrrolyl, pyrazolyl, imidazolyl, pyridyl,pyrazinyl, pyrimidinyl, pyridazinyl, indolyl, indazolyl, benzimidazolyl, quinolyl and isoquinolyl,
wherein said alkyl or cycloalkyl is unsubstituted or substituted with one or more
   (a) halogen,
   (b) —C₁₋₆alkyl,
   (c) —C₂₋₆alkenyl,
   (d) —C₁₋₆alkoxy,
   (e) —C₆₋₁₀ aryl,
   (f) hydroxyl, or
   (g) cyano,
and said heteroaryl is unsubstituted or substituted with one or more
   (a) —C₁₋₆alkyl,
   (b) —NR_{3f}R^{3g}, wherein R^{3f} and R^{3g} are selected from the group consisting of:
      (i) hydrogen,
      (ii) —C₁₋₆ alkyl,
      (iii) —C₁₋₆alkyl-C₆₋₁₀ aryl, wherein said aryl can be substituted or unsubstited with halogen, cyano, C₁₋₆ alkyl or C₁₋₆ alkoxy, or
      (iv) —C₁₋₆alkyl-NR^{7a}R^{7b},
   or N, R^{3f} and R^{3g} together form a 5 or 6 membered heterocyclic group, optionally containing an N, S or O atom in addition to the N atom attached to R^{3f} and R^{3g};
   and R^{3a}, R^{3b}, R^{3c}, R^{3d} and R^{3e} are selected from the group consisting of:
      (i) hydrogen,
      (ii) halogen,
      (iii) cyano,
      (iv) hydroxyl,
      (v) —C₁₋₆ alkyl,
      (vi) —O—R^{7a},
      (vii) —(C═O)—O—R⁸,
      (viii) —NR^{7a}—S(O)ₚOR^{7b},
      (ix) —NR^{7a}—S(O)ₚR^{7b},
      (x) —C₀₋₆alkyl —S(O)ₘR^{7a},
      (xi) —C(═O)—NR^{7a}R^{7b},
      (xii) —C(═O)—R⁸
      (xiii) —NH—C(═O)—R^{7a},
      (xiv) —C₀₋₆alkyl-NR^{7a}R^{7b},
      (xv) —N₃,
      (xvi) —NO₂,
   (xvii) C₆₋₁₀ aryl, wherein said aryl can be unsubstituted or substited with one or more
      (A) halogen
      (B) cyano,
      (C) —C₁₋₆ alkyl,
      (D) —C₁₋₆ alkoxy,
      (E) —C(═O)—O—R^{7a},
      (F) —C(═O)—R^{7a},
      (G) —NR^{7a}R^{7b},
      (H) —NR^{7a}—S(O)ₚ—R^{7b}
      (I) —NR^{7a}—C(═O)—R^{7b},
      (J) —NO₂
      (xvii) heteroaryl selected from the group consisting of furyl, pyranyl, benzofuranyl, isobenzofuranyl, chromenyl, thienyl, benzothiophenyl, pyrrolyl, pyrazolyl, imidazolyl, pyridyl, pyrazinyl, pyrimidinyl, pyridazinyl, indolyl, indazolyl, benzimidazolyl, quinolyl and isoquinolyl,
      wherein said heteroaryl is unsubstituted or substituted with one or more
         (A) —C₁₋₆ alkyl, or
         (B) —C₁₋₆ alkoxy,
      or R^{3c} and R^{3d} are linked together to form phenyl or the group —O—CH₂—O— or —CH═CH—CH═CH—;
or R² and R³ are linked to form a carbocyclic ring (A) 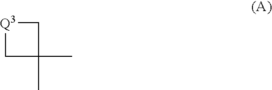
   wherein Q³ is selected from the group consisting of

(1) —CR^{7a}R^{7b}—,

(2) —CR^{7a}R^{7b}CR^{7c}R^{7d}—,

(3) —CR^{7a}═CR^{7b}—,

(4) —CR^{7a}R^{7b}CR^{7c}R^{7d}CR^{7e}R^{7f}—,

(5) —CR^{7a}═CR^{7b}CR^{7c}R^{7d}—, and

(6) —CR^{7a}R^{7b}CR^{7d}═CR^{7e}—;
R⁴ is selected from the group consisting of:

(1) hydrogen,

(2) halogen,

(3) —C₁₋₆alkyl,

(4) —C₂₋₆alkenyl,

(5) —C₂₋₆alkynyl,

(6) phenyl,

(7) benzyl, and

(8) heteroaryl selected from the group consisting of furyl, pyranyl, benzofuranyl, isobenzofuranyl, chromenyl, thienyl, benzothiophenyl, pyrrolyl, pyrazolyl, imidazolyl, pyridyl,pyrazinyl, pyrimidinyl, pyridazinyl, indolyl, indazolyl, benzimidazolyl, quinolyl and isoquinolyl,
wherein said alkyl, alkenyl, alkynyl and phenyl is unsubstituted or substituted with one or more
   (a) halogen,
   (b) cyano,
   (c) hydroxyl,
   (d) phenyl,
   (e) —C₁₋₆ alkyl,
   (f) —C₁₋₆ alkoxy,
   (g) —C(═O)—O—R^{7a},
   (h) —C(═O)—R^{7a},
   (i) —NR^{7a}R^{7b},
   (j) —NR^{7a}—S(O)ₚ—R^{7b},
   (k) —NR^{7a}—C(═O)—R^{7b},
   (l) —NO₂;
and said heteroaryl is unsubstituted or substituted with one or more
   (a) —C₁₋₆alkyl,
   (b) —C(═O)—O—R^{7a}
   (c) —C(═O)—R^{7a}
   (d) —NR^{3f}R^{3g}, wherein R^{3f} and R^{3g} selected from the group consisting of
      (i) hydrogen,
      (ii) —C₁₋₆ alkyl,
      (iii) —C₁₋₆alkyl-C₆₋₁₀ aryl, wherein said aryl can be substituted or unsubstited with halogen, cyano, C₁₋₆ alkyl or C₁₋₆ alkoxy, or
      (iv) —C₁₋₆alkyl-NR^{7a}R^{7b};
or R³ and R⁴ are joined together to form a 6-membered carbocyclic ring (B): 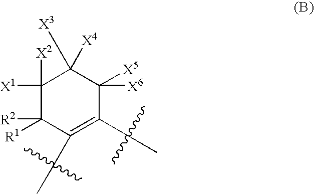
   provided that when R³ and R⁴ are joined together to form (B) then R¹ and R² are selected from the group consisting of hydrogen or C₁₋₆ alkyl, and X¹, X², X³, X⁴, X⁵ and X⁶ are selected from the group consisting of hydrogen, C₁₋₆ alkyl, C₃₋₆ cycloalkyl, cyano, alkylaryl or phenyl,
or R³ and R⁴ are joined together to form a 7-membered carbocyclic ring (C): 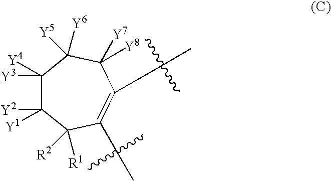
   provided that when R³ and R⁴ are joined together to form (C) then R¹ and R² are selected from the group consisting of hydrogen, C₁₋₆ alkyl or phenyl, or R¹ and R² can be linked together by the group —CH₂CH₂CH₂CH₂—; and Y¹, Y², Y³, Y⁴, Y⁵, Y⁶, Y⁷ and Y⁸ are selected from the group consisting of hydrogen, C₁₋₆ alkyl, C₃₋₆ cycloalkyl, cyano, alkylaryl or phenyl,
or R¹ and Y⁵, or R¹ and Y⁷, are linked together by —CH₂—,
or R¹ and Y¹, or Y¹ and Y³, are linked together to form a phenyl or cyclopentyl ring;
R^{7a}, R^{7b}, R^{7c}, R^{7d}, R^{7e} and R^{7f} are selected from the group consisting of:

(1) hydrogen,

(2) C₁₋₆ alkyl, and

(3) C₆₋₁₀ aryl,

wherein said alkyl or aryl is unsubstituted or substituted with one or more halogen, —C₁₋₆alkyl, —C₁₋₆alkoxy, hydroxyl or cyano;
R⁸ is selected from the group consisting of

(1) hydrogen,

(2) C₁₋₆ alkyl, and

(3) C₆₋₁₀ aryl, wherein said aryl is unsubstituted or substituted with one or more halogen, —C₁₋₆alkyl, —C₁₋₆alkoxy, hydroxy or cyano;
n is 0, 1, 2 or 3
m is 0 or 1;
p is 1 or 2;
and pharmaceutically acceptable salts thereof, and individual enantiomers and diastereomers thereof.

In one embodiment, the invention is directed to compounds of formula (I) wherein R² and R³ are not linked to form a cyclic group, and each of R¹, R² and R³ can be any of the groups defined above. In preferred groups, R³ is selected from the group consisting of:

(1) —C₁₋₆alkyl,

(2) —C₀₋₆alkyl-C₃₋₆cycloalkyl,

(3)

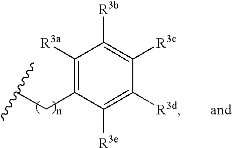

(4) —CH₂-heteroaryl.

In more preferred groups, R³ is 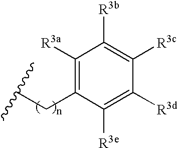
and n is 1. Preferably, R³ is in the (S) configuration, as depicted below: 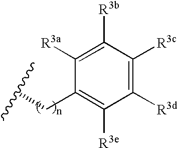
wherein n is 1. In even more preferred groups, R³ is in the (S) configuration as depicted above, n is 1 and R^{3a}, R^{3b}, R^{3c}, R^{3d} and R^{3e} are selected from the group consisting of:

(i) hydrogen,

(ii) halogen,

(iii) cyano,

(iv) hydroxyl,

(v) —C₁₋₆ alkyl,

(vi) —O—R^{7a}, and

(vii) —NO₂.

In preferred embodiments, R¹ is 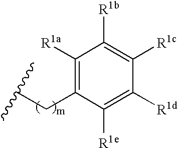
and m is 0. Preferably, R^{1a}, R^{1b}, R^{1d} and R^{1e} are hydrogen, and R^{1c} is selected from the group consisting of halogen, C₁₋₆ alkyl and C₁₋₆ alkoxy.

Thus, a preferred group of compounds is compounds of formula (II): 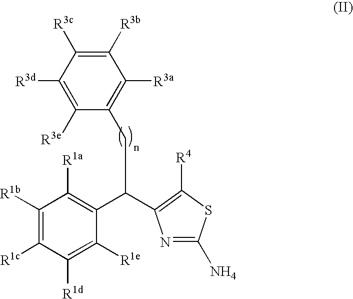
wherein R^{1a}, R^{1b}, R^{1c}, R^{1d}, R^{1e}, R^{3a}, R^{3b}, R^{3c}, R^{3d}, R^{3e}, R⁴ and n are as defined above.

In further preferred embodiments, R² is hydrogen. In other preferred embodiments, R⁴ is hydrogen.

In another embodiment, the invention is directed to compounds of formula (III) 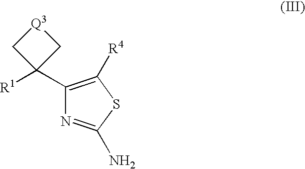
wherein R¹, R⁴ and Q³ are as defined above.

In preferred embodiments, Q³ is selected from the group consisting of

(1) —CR^{7a}R^{7b}—,

(2) —CR^{7a}R^{7b}CR^{7c}R^{7d}—, and

(3) —CR^{7a}R^{7b}CR^{7c}R^{7d}CR^{7e}R^{7f}—. Preferably, Q³ is selected from the group consisting of —CH₂CH₂— and —CH₂CH₂CH₂—.

In further preferred embodiments, R¹ is 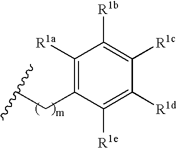
and m is 0. In more preferred embodiments, R^{1d} is selected from the group consisting of halogen, C₁₋₆ alkyl, C₁₋₆ alkoxy and cyano, and R^{1a}, R^{1b}, R^{1c} and R^{1e} are hydrogen. In other preferred embodiments, R^{1b} and R^{1d} are selected from the group consisting of halogen, C₁₋₆ alkyl, C₁₋₆ alkoxy and cyano, and R^{1a}, R^{1c} and R^{1e} are hydrogen.

In another embodiment, the invention is directed to compounds of formula (IV) 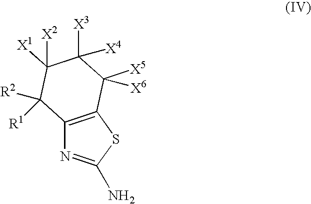
wherein R¹, R², X¹, X², X³, X⁴, X⁵ and X⁶ are as defined above.

Preferably, R¹ and R² are hydrogen, and X¹, X², X³, X⁴, X⁵ and X⁶ are selected from the group consisting of hydrogen, C₁₋₆ alkyl, cyano and phenyl.

In another embodiment, the invention is directed to compounds of formula (V) 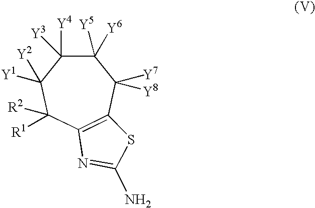
wherein R¹, R², Y¹, Y², Y³, Y⁴, Y⁵, Y⁶, Y⁷ and Y⁸ are as defined above.

Preferably, R¹ and R² are selected from the group consisting of hydrogen and phenyl, and Y¹, Y², Y³, Y⁴, Y⁵, Y⁶, Y⁷ and Y⁸ are selected from the group consisting of hydrogen, C₁₋₆ alkyl, cyano and phenyl.

Another embodiment of the present invention includes a compound which is selected from the title compounds of the following Examples and pharmaceutically acceptable salts thereof.

As used herein, the term “alkyl,” by itself or as part of another substituent, means a saturated straight or branched chain hydrocarbon radical having the number of carbon atoms designated (e.g., C₁₋₁₀ alkyl means an alkyl group having from one to ten carbon atoms). Preferred alkyl groups for use in the invention are C₁₋₆ alkyl groups, having from one to six carbon atoms. Exemplary alkyl groups include methyl, ethyl, n-propyl, isopropyl, n-butyl, isobutyl, tert-butyl, pentyl, hexyl, and the like.

A C₀ alkyl group, as used as part of another moiety, for example C₀₋₆ alkyl-C₃₋₆ cycloalkyl, represents a bond. Hence, if R³ is defined herein as C₀ alkyl-C₃₋₆ cycloalkyl, R³ is a -C₃₋₆ cycloalkyl group.

As used herein, the term “alkoxy,” by itself or as part of another substituent, means the group —O— alkyl, wherein alkyl is defined above, having the number of carbon atoms designated (e.g., C₁₋₁₀alkoxy means an alkoxy group having from one to ten carbon atoms). Preferred alkoxy groups for use in the invention are C₁₋₆ alkoxy groups. Exemplary preferred alkoxy groups include methoxy, ethoxy, propoxy, butoxy, sec-butoxy and pentoxy.

As used herein, the term “alkenyl,” by itself or as part of another substituent, means a straight or branched chain hydrocarbon radical having a single carbon-carbon double bond and the number of carbon atoms designated (e.g., C₂₋₁₀ alkenyl means an alkenyl group having from two to ten carbon atoms). Preferred alkenyl groups for use in the invention are C₂₋₆ alkenyl groups, having from two to six carbon atoms. Exemplary alkenyl groups include ethenyl and propenyl.

As used herein, the term “cycloalkyl,” by itself or as part of another substituent, means a saturated cyclic hydrocarbon radical having the number of carbon atoms designated (e.g., C₃₋₆ cycloalkyl means a cycloalkyl group having from three to eight carbon atoms). Exemplary cycloalkyl groups include cyclopropyl, cyclobutyl, cyclopentyl, cyclohexyl and the like.

As used herein, the term “aryl,” by itself or as part of another substituent, means an aromatic or cyclic radical having the number of carbon atoms designated (e.g., C₆₋₁₀ aryl means an aryl group having from six to ten carbons atoms). Preferred aryl groups for use in the invention include phenyl and naphthyl.

The term “halo” or “halogen” includes fluoro, chloro, bromo and iodo.

As used herein, the term “heteroaryl,” by itself or as part of another substituent, means an aromatic cyclic group having at least one ring heteroatom (O, N or S). Exemplary heteroaryl groups for use in the invention include furyl, pyranyl, benzofuranyl, isobenzofuranyl, chromenyl, thienyl, benzothiophenyl, pyrrolyl, pyrazolyl, imidazolyl, pyridyl,pyrazinyl, pyrimidinyl, pyridazinyl, indolyl, indazolyl, benzimidazolyl, quinolyl and isoquinolyl.

When a heteroaryl group as defined herein is substituted, the substituent may be bonded to a ring carbon atom of the heteroaryl group, or on a ring heteroatom (i.e., a nitrogen, oxygen or sulfur), which has a valence which permits substitution. Preferably, the substituent is bonded to a ring carbon atom.

Some of the compounds of the instant invention have at least one asymmetric center. Additional asymmetric centers may be present depending upon the nature of the various substituents on the molecule. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all such isomeric forms of these compounds.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art.

Alternatively, any enantiomer of a compound may be obtained by stereoselective synthesis using optically pure starting materials or reagents of known configuration by methods well known in the art.

The compounds claimed in this invention can be prepared according to the following general procedure methods A-D, and the specific examples 1-6.

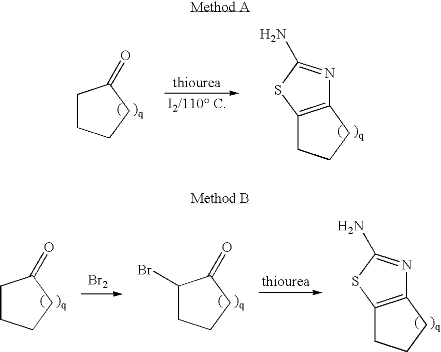

Methods A and B may be used to obtain compounds of formula (1) wherein R³ and R⁴ are linked together to form a C₆ carbocyclic ring of formula (B) (when q is 2), or compounds of formula (I) wherein R³ and R⁴ are linked together to form a C₇ carbocyclic ring of formula (C) (when q is 3). The aminothiazole ring system in method A may be formed in a single step by heating a neat mixture of an appropriately substituted ketone containing an (x-methylene group in a sealed tube with thiourea and iodine. An alternative two-step procedure is outlined in method B and involves the formation of an α-haloketone from the starting ketone with an halogenating agent such as N-bromosuccinimide or bromine in an appropriate solvent.

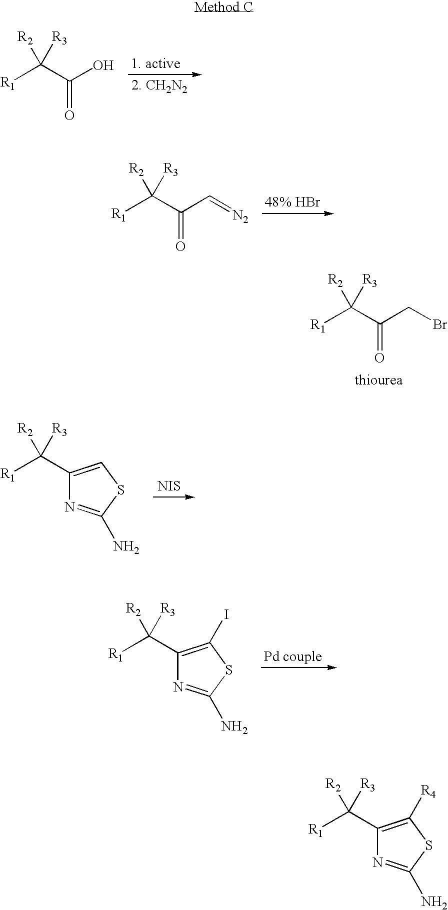

Method C forms compounds wherein neither R² nor R³ are linked to R⁴ to form a cyclic group, and each of R¹, R² and R³ can be any of the groups defined above. Methods C and D may also be used to form compounds wherein R² and R³ are linked to form a carbocyclic ring. Method C requires an appropriately substituted carboxylic acid as the starting material. The carboxyl group is converted to an activated carboxy functional group, such as an acid halide or a mixed anhydride, by known methods. The activated group is displaced by ethereal diazomethane at ambient temperature over a period of up to 72h, and the subsequently formed α-diazoketone is converted to an-α-haloketone by exposure to a solution of HCl gas or aqueous hydrobromic acid. The thiazole ring system can be formed by stirring the haloketone in a solvent such as methanol or ethanol with at least one equivalent of thiourea with or without an acid scavenger such as sodium bicarbonate. Further functionalization of the thiazole ring may be effected by halogenation at the 5 position by reaction with an halogenating agent such as N-iodosuccinimide in acetonitrile. Carbon-carbon bond formation can occur by a palladium mediated coupling reaction of the halothiazole with an appropriate organometallic agent.

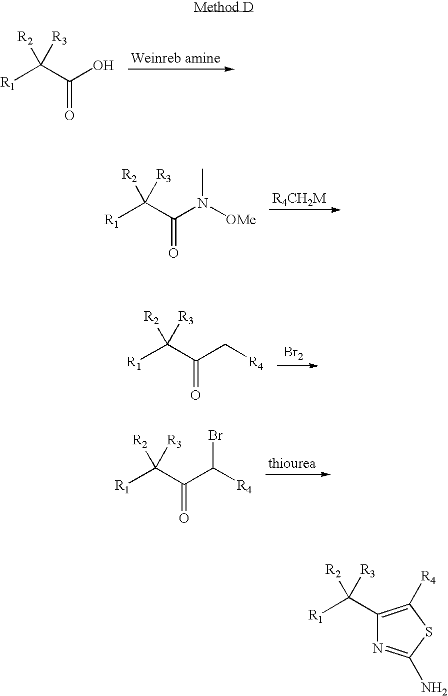

Alternatively, in Method D the R⁴ group may be introduced starting from a carboxylic acid and converting it to the corresponding Weinreb amide by known methods. Ketone formation can occur by reacting the aforementioned amide with an organometallic agent, such as an organolithium or Grignard reagent, in a solvent such as THF or ether at −70° C. to room temperature. Halogenation can be effected with a reagent such as bromine in chloroform at about 50° C. The thiazole ring system can be formed by stirring the haloketone in a solvent, such as methanol or ethanol, with at least one equivalent of thiourea with or without an acid scavenger, such as sodium bicarbonate.

The term “substantially pure” means that the isolated material is at least 90% pure, and preferably 95% pure, and even more preferably 99% pure as assayed by analytical techniques known in the art.

The term “pharmaceutically acceptable salts” refers to salts prepared from pharmaceutically acceptable non-toxic bases or acids including inorganic or organic bases and inorganic or organic acids. The compounds of the invention may be mono, di or tris salts, depending on the number of acid functionalities present in the free base form of the compound. Free bases and salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, trifluoroacetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, trifluoroacetic, maleic, phosphoric, sulfuric, fumaric, and tartaric acids.

The present invention is directed to the use of the compounds disclosed herein as inhibitors of β-secretase enzyme activity or β-site amyloid precursor protein-cleaving enzyme (“BACE”) activity, in a patient or subject such as a mammal in need of such inhibition, comprising the administration of an effective amount of the compound. The compounds of the present invention are useful for treating Alzheimer's disease by inhibiting the activity of β-secretase or BACE, thus preventing the formation of insoluble Aβ and arresting the production of Aβ. The terms “P-secretase enzyme,” “β-site amyloid precursor protein-cleaving enzyme,” and “BACE” are used interchangeably in this specification. In addition to humans, a variety of other mammals can be treated according to the method of the present invention.

The present invention is further directed to a method for the manufacture of a medicament or a composition for inhibiting β-secretase enzyme activity in humans and animals comprising combining a compound of the present invention with a pharmaceutical carrier or diluent.

The compounds of the present invention have utility in treating, ameliorating, controlling or reducing the risk of Alzheimer's disease. For example, the compounds may be useful for the prevention of dementia of the Alzheimer's type, as well as for the treatment of early stage, intermediate stage or late stage dementia of the Alzheimer's type. The compounds may also be useful in treating, ameliorating, controlling or reducing the risk of diseases mediated by abnormal cleavage of amyloid precursor protein (also referred to as APP), and other conditions that may be treated or prevented by inhibition of β-secretase. Such conditions include mild cognitive impairment, Trisomy 21 (Down Syndrome), cerebral amyloid angiopathy, degenerative dementia, Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, Down syndrome, pancreatitis, inclusion body myositis, other peripheral amyloidoses, diabetes and atherosclerosis.

The compounds of the present invention are also useful in the inhibition of HIV protease, the prevention of infection by HIV, the treatment of infection by HIV and in the treatment of AIDS and/or ARC, when used as compounds or pharmaceutically acceptable salts or hydrates (when appropriate) thereof, optionally as pharmaceutical composition ingredients, and optionally in combination with other HIV protease inhibitors, antivirals, anti-infectives, immunomodulators, antibiotics or vaccines.

The present invention is further directed to a method for the manufacture of a medicament or a composition for inhibiting HIV protease activity in humans and animals comprising combining a compound of the present invention with a pharmaceutical carrier or diluent.

The compounds of the present invention may be used in combination with one or more other drugs in the treatment of diseases or conditions for which the compounds of the present invention have utility, where the combination of the drugs together are safer or more effective than either drug alone. Additionally, the compounds of the present invention may be used in combination with one or more other drugs that treat, prevent, control, ameliorate, or reduce the risk of side effects or toxicity of the compounds of the present invention. Such other drugs may be administered, by a route and in an amount commonly used therefor, contemporaneously or sequentially with the compounds of the present invention. Accordingly, the pharmaceutical compositions of the present invention include those that contain one or more other active ingredients, in addition to the compounds of the present invention. The combinations may be administered as part of a unit dosage form combination product, or as a kit or treatment protocol wherein one or more additional drugs are administered in separate dosage forms as part of a treatment regimen.

Examples of combinations of the compounds of the present invention with other drugs in either unit dose or kit form include combinations with anti-Alzheimer's agents, for example other beta-secretase inhibitors or gamma-secretase inhibitors; HMG-CoA reductase inhibitors; NSAIDs including ibuprofen; vitamin E; anti-amyloid antibodies, including anti-amyloid humanized monoclonal antibodies; CB-1 receptor antagonists or CB-1 receptor inverse agonists; antibiotics such as doxycycline and rifampin; N-methyl-D-aspartate (NMDA) receptor antagonists, such as memantine; cholinesterase inhibitors such as galantamine, rivastigmine, donepezil, and tacrine; growth hormone secretagogues such as ibutamoren, ibutamoren mesylate, and capromorelin; histamine H₃ antagonists; AMPA agonists; PDE IV inhibitors; GABA_{A} inverse agonists; neuronal nicotinic agonists; or other drugs that affect receptors or enzymes that either increase the efficacy, safety, convenience, or reduce unwanted side effects or toxicity of the compounds of the present invention. The foregoing list of anti-Alzheimer's agents suitable for combinations is illustrative only and not intended to be limiting in any way.

The present invention is also directed to combinations of the compounds of the invention with one or more agents useful in the treatment of AIDS. For example, the compounds of this invention may be effectively administered, whether at periods of pre-exposure and/or post-exposure, in combination with effective amounts of the AIDS antivirals, imunomodulators, antiinfectives, or vaccines. Suitable anti-viral agents which may be used in combination with the compounds of the invention include non-nucleoside HIV reverse transcriptase inhibitors, nucleoside HIV reverse transcriptase inhibitors, CCR5 receptor antagonists, HIV integrase inhibitors and cytochrome P450 monooxygenase inhibitor (e.g., indinavir or ritonavir or a pharmaceutically acceptable salt thereof).

Examples of particular anti-AIDS or anti-HIV agents (including antivirals, immunomodulators, antiinfecives, and other agents) which are suitable for combinations are listed in Tables 1-4, as follows:

TABLE 1
ANTIVIRALS

| DRUG NAME | MANUFACTURER | INDICATION |
|---|---|---|
| Abacavir | GlaxoSmithKline | HIV infection, AIDS, ARC |
| | (ZIAGEN ™) | (nRTI) |
| Abacavir + | GlaxoSmithKline | HIV infection, AIDS, ARC |
| lamivudine + | (TRIZIVIR ™) | (nRTI) |
| zidovudine | | |
| Amprenavir | GlaxoSmithKline | HIV infection, AIDS, |
| | (AGENERASE ™) | ARC (PI) |
| ACH 126443 | Achillion Pharm. | HIV infection, AIDS, ARC |
| | | (nRTI) |
| Acemannan | Carrington Labs | ARC |
| | (Irving, TX) | |
| Acyclovir | GlaxoSmithKline | HIV infection, AIDS, |
| | (ZOVIRAX ™) | ARC, in combination with |
| | | AZT |
| AD-439 | Tanox Biosystems | HIV infection, AIDS, ARC |
| AD-519 | Tanox Biosystems | HIV infection, AIDS, ARC |
| Adefovir | Gilead Sciences | HIV infection, AIDS, |
| dipivoxil | | ARC (RTI) |
| AL-721 | Ethigen | ARC, PGL |
| | (Los Angeles, CA) | HIV positive, AIDS |
| Alpha | GlaxoSmithKline | Kaposi's sarcoma, |
| Interferon | | HIV in combination |
| | | w/Retrovir |
| AMD3100 | AnorMed | HIV infection, AIDS, ARC |
| Ansamycin | Adria | ARC |
| LM 427 | Laboratories | |
| | (Dublin, OH) | |
| | Erbamont | |
| | (Stamford, CT) | |
| Antibody which | Advanced | AIDS, ARC |
| neutralizes pH | Biotherapy | |
| labile alpha | Concepts | |
| aberrant | (Rockville, MD) | |
| Interferon | | |
| AR177 | Aronex Pharm | HIV infection, AIDS, ARC |
| Atazanavir | Bristol-Myers- | HIV infection, AIDS, |
| | Squibb | ARC (PI) |
| | (REYATAZ ™) | |
| beta-fluoro-ddA | Nat'l Cancer | AIDS-associated diseases |
| | Institute | |
| BMS-232623 | Bristol-Myers | HIV infection, AIDS, ARC |
| (CGP-73547) | Squibb/Novartis | (PI) |
| BMS-234475 | Bristol-Myers | HIV infection, AIDS, ARC |
| (CGP-61755) | Squibb/Novartis | (PI) |
| CI-1012 | Warner-Lambert | HIV-1 infection |
| Cidofovir | Gilead Sciences | CMV retinitis, herpes, |
| | (VISTIDE ™) | papillomavirus |
| Curdlan sulfate | AJI Pharma USA | HIV infection |
| Cytomegalovirus | MedImmune | CMV retinitis |
| Immune | | |
| Globulin | | |
| Delavirdine | Pfizer | HIV infection, AIDS, ARC |
| | (RESCRIPTOR ™) | (RTI) |
| Dextran Sulfate | Ueno Fine Chem. | AIDS, ARC, HIV |
| | Ind. Ltd. | positive asymptomatic |
| | (Osaka, Japan) | |
| Didanosine (ddI, | Bristol-Myers | HIV infection, AIDS, ARC; |
| 2′,3′- | Squibb | combination with AZT/d4T |
| Dideoxyinosine) | (VIDEX ™) | |
| DPC 681, DPC 684 | Bristol | HIV infection, AIDS, |
| | Myers Squibb | ARC (PI) |
| DPC 961, DPC 083 | Bristol | HIV infection, AIDS, ARC |
| | Myers Squibb | (nnRTI) |
| Efavirenz | DuPont | HIV infection, AIDS, ARC |
| | (SUSTIVA ™), | (nnRTI) |
| | Merck | |
| | (STOCRIN ™) | |
| EL10 | Elan Corp. | HIV infection |
| Emtricitabine | Gilead Sciences | HIV infection, AIDS, ARC |
| (FTC) | (COVIRACIL ™) | (nRTI) |
| Emvirine | Gilead Sciences | HIV infection, AIDS, ARC |
| | (COACTINON ™) | (nNRTI) |
| Enfuvirtide | Roche | HIV infection, AIDS, ARC |
| | (FUZEON ™) | (fusion inhibitor) |
| Famciclovir | Novartis | herpes zoster, herpes |
| | (FAMVIR ™) | simplex |
| Ganciclovir | Roche | sight threatening CMV |
| | (CYTOVENE ™) | peripheral CMV |
| | | retinitis |
| GS 840 | Gilead | HIV infection, AIDS, ARC |
| | | (RTI) |
| HBY097 | Hoechst | HIV infection, AIDS, ARC |
| | Marion Roussel | (nnRTI) |
| Hypericin | VIMRx Pharm. | HIV infection, AIDS, ARC |
| Recombinant | Triton | AIDS, Kaposi's |
| Human | Biosciences | sarcoma, ARC |
| Interferon | (Almeda, CA) | |
| Beta | | |
| Interferon | Interferon | ARC, AIDS |
| alfa-n3 | Sciences | |
| Indinavir | Merck | HIV infection, AIDS, ARC, |
| | (CRIXIVAN ™) | asymptomatic HIV positive, |
| | | also in combination with |
| | | AZT/ddI/ddC |
| ISIS 2922 | ISIS | CMV retinitis |
| | Pharmaceuticals | |
| JE2147/AG1776 | Agouron | HIV infection, AIDS, ARC |
| | | (protease inhibitor) |
| KNI-272 | Nat'l | HIV-assoc. diseases |
| | Cancer Institute | |
| Lamivudine, 3TC | GlaxoSmithKline | HIV infection, AIDS, ARC |
| | (EPIVIR ™) | (RTI); also with AZT |
| Lamivudine + | GlaxoSmithKline | HIV infection, AIDS, ARC |
| zidovudine | (COMBIVIR ™) | (nRTI) |
| Lobucavir | Bristol-Myers | CMV infection |
| | Squibb | |
| Lopinavir | Abbott | HIV infection, AIDS, |
| (ABT-378) | | ARC (PI) |
| Lopinavir + | Abbott | HIV infection, AIDS, |
| ritonavir | (KALETRA ™) | ARC (PI) |
| Mozenavir | AVID | HIV infection, AIDS, |
| (DMP-450) | (Camden, NJ) | ARC (PI) |
| Nelfinavir | Pfizer | HIV infection, AIDS, |
| | (VIRACEPT ™) | ARC (PI) |
| Nevirapine | Boehringer | HIV infection, AIDS, |
| | Ingelheim | ARC (nnRTI) |
| | (VIRAMUNE ™) | |
| Novapren | Novaferon | HIV inhibitor |
| | Labs, Inc. | |
| | (Akron, OH) | |
| Peptide T | Peninsula Labs | AIDS |
| Octapeptide | (Belmont, CA) | |
| Sequence | | |
| PRO 140 | Progenics | HIV infection, AIDS, |
| | | ARC (CCR5 co-receptor |
| | | inhibitor) |
| PRO 542 | Progenics | HIV infection, AIDS, |
| | | ARC (attachment |
| | | inhibitor) |
| Probucol | Vyrex | HIV infection, AIDS |
| RBC-CD4 | Sheffield Med. | HIV infection, AIDS, |
| | Tech (Houston | ARC |
| | TX) | |
| Ribavirin | Viratek/ICN | asymptomatic HIV |
| | (VIRAZOLE ™) | positive, LAS, ARC |
| | (Costa Mesa, CA) | |
| Ritonavir | Abbott | HIV infection, AIDS, |
| | | ARC (PI) |
| Saquinavir | Roche | HIV infection, AIDS, |
| | (INVIRASE ™) | ARC (PI) |
| Stavudine (d4T, | Bristol-Myers | HIV infection, AIDS, |
| Didehydrodeoxy- | Squibb | ARC (nRTI) |
| Thymidine) | (ZERIT ™) | |
| T-1249 | Trimeris | HIV infection, AIDS, |
| | | ARC (fusion inhibitor) |
| TAK-779 | Takeda | HIV infection, AIDS, |
| | | ARC (injectabe CCR5 |
| | | receptor antagonist) |
| Tenofovir | Gilead | HIV infection, AIDS, |
| | Sciences | ARC (nRTI) |
| | (VIREAD ™) | |
| Tipranavir | Boehringer | HIV infection, AIDS, |
| | Ingelheim | ARC (PI) |
| TMC 120 & | Tibotec | HIV infection, AIDS, |
| TMC 125 | | ARC (nnRTI) |
| TMC 126 | Tibotec | HIV infection, AIDS, |
| | | ARC (PI) |
| Trisodium | Astra | CMV retinitis, HIV |
| Phosphonoformate | Pharmaceuticals | infection, other CMV |
| | | infections |
| Valaciclovir | GlaxoSmithKline | Genital HSV & CMV |
| | | infections |
| VX-478 | Vertex | HIV infection, AIDS, ARC |
| Zalcitabine (ddC, | Roche (HIVID ™) | HIV infection, AIDS, ARC |
| 2′,3′- | | |
| Dideoxycytidine) | | |
| Zidovudine; | GlaxoSmithKline | HIV infection, AIDS, ARC, |
| AZT | (RETROVIR ™) | Kaposi's sarcoma, in |
| | | combination with other |
| | | therapies |

TABLE 2
Immunomodulators

| DRUG NAME | MANUFACTURER | INDICATION |
|---|---|---|
| AS-101 | Wyeth | AIDS |
| Bropirimine | Pfizer | advanced AIDS |
| CL246, 738 | American Cyanamid | AIDS, Kaposi's |
| | Lederle Labs | sarcoma |
| Etanercept | Immunex Corp. | Rheumatoid arthritis |
| | (ENBREL ™) | |
| FP-21399 | Fuki ImmunoPharm | blocks HIV fusion with CD4+ |
| | | cells |
| Gamma | Genentech | ARC, in combination |
| Interferon | | w/TNF (tumor necrosis factor) |
| Granulocyte | Genetics | AIDS |
| Macrophage | Institute/Sandoz | |
| Colony | | |
| Stimulating | | |
| Factor | | |
| Granulocyte | Hoechst- | AIDS |
| Macrophage | Roussel/Immunex | |
| Colony | | |
| Stimulating | | |
| Factor | | |
| Granulocyte | Schering-Plough | AIDS, combination |
| Macrophage | | w/AZT |
| Colony | | |
| Stimulating | | |
| Factor | | |
| HIV Core | Rorer | seropositive HIV |
| Particle | | |
| Immunostimulant | | |
| IL-2 | Cetus | AIDS, in combination |
| Interleukin-2 | | w/AZT |
| IL-2 | Roche/Immunex | AIDS, ARC, HIV, in |
| Interleukin-2 | | combination w/AZT |
| IL-2 | Chiron | AIDS, increase in CD4 cell |
| Interleukin-2 | | counts |
| (aldeslukin) | | |
| Infliximab | Centocor | Rheumatoid arthritis, |
| | (REMICADE ™) | Crohn's Disease |
| Immune Globulin | Cutter Biological | pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol | Merieux Institute | AIDS, ARC |
| Diethyl | | |
| Dithio | | |
| Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl- | | |
| Tripeptide | | |
| Granulocyte | Amgen | AIDS, in combination |
| Colony | | w/AZT |
| Stimulating | | |
| Factor | | |
| Remune | Immune | immunotherapeutic |
| | Response Corp. | |
| rCD4 | Genentech | AIDS, ARC |
| Recombinant | | |
| Soluble | | |
| Human CD4 | | |
| Recombinant | Biogen | AIDS, ARC |
| Soluble | | |
| Human CD4 | | |
| Interferon | Roche | Kaposi's sarcoma |
| Alfa 2 a | | AIDS, ARC, in |
| | | combination w/AZT |
| Thymopentin | Immunobiology | HIV infection |
| | Research | |
| | Institute | |
| | (Annandale, NJ) | |
| Tumor Necrosis | Genentech | ARC, in combination w/gamma |
| Factor; TNF | | Interferon |

TABLE 3
ANTI-INFECTIVES

| DRUG NAME | MANUFACTURER | INDICATION |
|---|---|---|
| Clindamycin with | Pfizer | PCP |
| Primaquine | | |
| Fluconazole | Pfizer | cryptococcal |
| | | meningitis, candidiasis |
| Nystatin | Bristol Myers | prevention of |
| (pastille) | Squibb Corp. | oral candidiasis |
| Eflornithine | Aventis (ORNIDYL ™) | PCP |
| Pentamidine | various | PCP |
| Isethionate | | |
| Trimethoprim | various | antibacterial |
| Piritrexim | Burroughs Wellcome | PCP treatment |
| Spiramycin | Rhone-Poulenc | cryptosporidial |
| | | diarrhea |
| Intraconazole- | Janssen | histoplasmosis; |
| R51211 | Pharmaceuticals | cryptococcal |
| | | meningitis |
| Trimetrexate | Warner-Lambert | PCP |

TABLE 4
OTHER

| DRUG NAME | MANUFACTURER | INDICATION |
|---|---|---|
| Daunorubicin | Various | Karposi's sarcoma |
| Recombinant Human | Ortho Pharm. Corp. | severe anemia |
| Erythropoietin | | assoc. with AZT therapy |
| Recombinant Human | Serono | AIDS-related wasting, |
| Growth Hormone | | cachexia |
| Megestrol Acetate | Bristol-Myers Squibb | treatment of |
| | | anorexia assoc. w/AIDS |
| Testosterone | Various | AIDS-related wasting |
| Total Enteral | Norwich Eaton | diarrhea and |
| Nutrition | Pharmaceuticals | malabsorption |
| | | related to AIDS |

AIDS = Acquired Immune Deficiency Syndrome
ARC = AIDS related complex
PI = protease inhibitor
RTI = reverse transcriptase inhibitor
nRTI = nucleoside reverse transcriptase inhibitor
nnRTI = non-nucleoside reverse transcriptase inhibitor
PGL = persistent generalized lymphadenopathy
PCP = pneumocystis carinii pneumonia
CMV = cytomegalovirus

It will be understood that the scope of combinations of the compounds of this invention with AIDS antivirals, immunomodulators, anti-infectives or vaccines is not limited to the list in Tables 1-4 above, but includes in principle any combination with any pharmaceutical composition useful for the treatment of AIDS.

One suitable combination is a compound of the present invention and a nucleoside inhibitor of HIV reverse transcriptase such as AZT, 3TC, ddC, or ddI. Another suitable combination is a compound of the present invention and a non-nucleoside inhibitor of HIV reverse transcriptase, such as efavirenz, and optionally a nucleoside inhibitor of HIV reverse transcriptase, such as AZT, 3TC, ddC or ddI.

Still another suitable combination is any one of the combinations in the preceding paragraph, further comprising an additional HIV protease inhibitor such as indinavir, nelfinavir, ritonavir, saquinavir, amprenavir, or abacavir. An aspect of this combination is the combination wherein the additional inhibitor of HIV protease is the sulfate salt of indinavir. Another aspect of this combination is the combination in which the additional protease inhibitor is selected from nelfinavir and ritonavir. Still another aspect of this combination is the combination in which the additional inhibitor of HIV protease is saquinavir, which is typically administered in a dosage of 600 or 1200 mg tid.

Other suitable combinations include a compound of the present invention with the following (1) efavirenz, optionally with AZT and/or 3TC and/or ddl and/or ddC, and optionally with indinavir; (2) any of AZT and/or ddI and/or ddC and/or 3TC, and optionally with indinavir; (3) d4T and 3TC and/or AZT; (4) AZT and 3TC; and (5) AZT and d4T.

Another aspect of the present invention is co-administration of a compound of the present invention with an inhibitor of cytochrome P450 monooxygenase in an amount effective to improve the pharmacokinetics of the compound. Compounds of the invention can be metabolized, at least in part, by cytochrome P450 (CYP3A4). Co-administration of compounds of the invention with a cytcochrome P450 inhibitor can improve the pharmacokinetic profile of the compound in subjects (e.g., humans); i.e., co-administration can increase Cmax (the maximum plasma concentration of the compound), AUC (area under the curve of plasma concentration of the compound versus time), and/or the half-life of the compound. Suitable P450 inhibitors include, but are not limited to, indinavir and ritonavir. It is to be understood that the primary role of indinavir and ritonavir in this circumstance is as a pharmacokinetic modulator and not as a protease inhibitor; i.e., an amount of indinavir or ritonavir which is effective for improving the pharmacokinetics of the compound can provide a secondary or even negligible contribution to the antiviral effect. Improvements in the pharmacokinetic profile have been observed for compounds of the present invention, when co-dosed with P450-inhibiting amounts of either ritonavir or indinavir.

The composition of the present invention can also be administered in combination with an HIV integrase inhibitor such as a compound described in WO 99/62520, WO 99/62513, or WO 99/62897. The composition of the present invention can also be administered in combination with a CCR⁵ receptor antagonist, such as a compound described in WO 00/59502 or WO 00/59503.

In the above-described combinations, the compound of the present invention and other active agents may be administered together or separately. In addition, the administration of one agent may be prior to, concurrent with, or subsequent to the administration of other agent(s). These combinations may have unexpected or synergistic effects on limiting the spread and degree of infection of HIV.

The subject or patient to whom the compounds of the present invention is administered is generally a human being, male or female, in whom inhibition of β-secretase enzyme or HIV protease activity is desired, but may also encompass other mammals, such as dogs, cats, mice, rats, cattle, horses, sheep, rabbits, monkeys, chimpanzees or other apes or primates, for which aspartyl protease inhibition (in particular, inhibition of β-secretase enzyme activity and/or inhibition of HIV protease) or treatment of the above noted disorders is desired.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Other pharmaceutical compositions include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

The pharmaceutical compositions may be in the form of a sterile injectable aqueous or oleaginous suspension, which may be formulated according to the known art, or may be administered in the form of suppositories for rectal administration of the drug.

The compounds of the present invention may also be administered by inhalation, by way of inhalation devices known to those skilled in the art, or by a transdermal patch.

By “pharmaceutically acceptable” it is meant the carrier, diluent or excipient must be compatible with the other ingredients of the formulation and not deleterious to the recipient thereof.

The terms “administration of“ or “administering a” compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” means the amount of the subject compound that will elicit the biological or medical response of a tissue, system, animal or human that is being sought by the researcher, veterinarian, medical doctor or other clinician. As used herein, the term “treatment” refers to the treatment of the mentioned conditions, particularly in a patient who demonstrates symptoms of the disease or disorder.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The compositions containing compounds of the present invention may conveniently be presented in unit dosage form and may be prepared by any of the methods well known in the art of pharmacy. The term “unit dosage form” is taken to mean a single dose wherein all active and inactive ingredients are combined in a suitable system, such that the patient or person adminstering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples of unit dosage forms.

The compositions containing compounds of the present invention may conveniently be presented as a kit, whereby two or more components, which may be active or inactive ingredients, carriers, diluents, and the like, are provided with instructions for preparation of the actual dosage form by the patient or person adminstering the drug to the patient. Such kits may be provided with all necessary materials and ingredients contained therein, or they may contain instructions for using or making materials or components that must be obtained independently by the patient or person administering the drug to the patient.

When treating, ameliorating, controlling or reducing the risk of Alzheimer's disease, AIDS or other diseases for which compounds of the present invention are indicated, generally satisfactory results are obtained when the compounds of the present invention are administered at a daily dosage of from about 0.1 mg to about 100 mg per kg of animal body weight, preferably given as a single daily dose or in divided doses two to six times a day, or in sustained release form. The total daily dosage is from about 1.0 mg to about 2000 mg, preferably from about 0.1 mg to about 20 mg per kg of body weight. In the case of a 70 kg adult human, the total daily dose will generally be from about 7 mg to about 1,400 mg. This dosage regimen may be adjusted to provide the optimal therapeutic response. The compounds may be administered on a regimen of 1 to 4 times per day, preferably once or twice per day.

Specific dosages of the compounds of the present invention, or pharmaceutically acceptable salts thereof, for administration include 1 mg, 5 mg, 10 mg, 30 mg, 80 mg, 100 mg, 150 mg, 300 mg and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 100 mg active ingredient. Specific pharmaceutical compositions useful for treatment may comprise about 1 mg, 5 mg, 10 mg, 30 mg, 80 mg, 100 mg, 150 mg, 300 mg and 500 mg of active ingredient.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The utility of the compounds in accordance with the present invention as inhibitors of β-secretase enzyme activity may be demonstrated by methodology known in the art. β-secretase enzyme inhibition is determined as follows:

ECL Assay: A homogeneous end point electrochemiluminescence (ECL) assay was used with a biotinylated BACE substrate. The Km of the substrate is greater than 100 μM and can not be determined due to the limit of solubility of the substrate. A typical reaction contained approximately 0.1 nM enzyme, 0.25 μM of the substrate, and buffer (50 mM NaOAc, pH 4.5, 0.1 mg/ml BSA, 0.2% CHAPS, 15 mM EDTA and 1 mM deferoxamine) in a total reaction volume of 100 μl. The reaction proceeded for 30 min and was then stopped by the addition of 25 μL of 1 M Tris-HCl, pH 8.0. The resulting enzymatic product was assayed by adding a ruthenylated antibody which specifically recognized the C-terminal residue of the product. Streptavidin coated magnetic beads were added into the solution and the samples were subjected to M-384 (Igen Inc., Gaithersburg, Md.) analysis. Under these conditions, less than 10% of substrate was processed by BACE 1. The enzyme used in these studies was soluble (transmembrane domain and cytoplasmic extension excluded) human protein produced in a baculovirus expression system. To measure the inhibitory potency for compounds, solutions of inhibitor in DMSO (12 concentrations of the inhibitors were prepared starting from 100 μM with three fold series dilution) were included in the reaction mixture (final DMSO concentration is 10%). All experiments were conducted at room temperature using the standard reaction conditions described above. To determine the IC50 of the compound, a four parameter equation is used for curve fitting. The errors in reproducing the dissociation constants are typically less than two-fold.

HPLC assay: A homogeneous end point HPLC assay was used with the substrate (coumarin-CO-REVNFEVEFR), which is cleaved by BACE 1 to release the N-terminal fragment attached with coumarin. The Km of the substrate is greater than 100 μM and can not be determined due to the limit of solubility of the substrate. A typical reaction contains approximately 2 nM enzyme, 1.0 μM of the substrate, and buffer (50 mM NaOAc, pH 4.5, 0.1 mg/ml BSA, 0.2% CHAPS, 15 mM EDTA and 1 mM deferoxamine) in a total reaction volume of 100 μl. The reaction proceeded for 30 min and was stopped by the addition of 25 μL of 1 M Tris-HCl, pH 8.0. The resulting reaction mixture was loaded on the HPLC and the product was separated from substrate with 5 min linear gradient. Under these conditions, less than 10% of substrate was processed by BACE 1. The enzyme used in these studies was soluble (transmembrane domain and cytoplasmic extension excluded) human protein produced in a baculovirus expression system. To measure the inhibitory potency for compounds, solutions of inhibitor in DMSO (12 concentrations of the inhibitors were prepared and the concentration rage was dependent on the potency predicted by ECL) were included in the reaction mixture (final DMSO concentration is 10%). All experiments were conducted at room temperature using the standard reaction conditions described above. To determine the IC₅₀ of the compound, a four parameter equation is used for curve fitting. The errors in reproducing the dissociation constants are typically less than two-fold.

In particular, the compounds of the following examples had activity in inhibiting the beta-secretase enzyme in the aforementioned assays, generally with an IC₅₀ from about 1 nM to 100 μM. Such a result is indicative of the intrinsic activity of the compounds in use as inhibitors of beta-secretase enzyme activity.

The utility of the compounds of the present invention as inhibitors of HIV protease may be demonstrated by methodology known in the art. HIV protease inhibition is determined as follows:

HIV Protease Assay: All enzyme-catalyzed reactions were performed under initial velocity and steady-state conditions. Specifically, conditions for the enzyme catalyzed hydrolysis of the MA/CA cleavage site peptide VSQN-(-naphthylalanine)-PIV were established with respect to time and enzyme concentration to yield linear initial velocity data. The enzyme concentrations used in the assay were as follows: wild-type, 5 pM; A-44 and A-44r, 200 pM; V-18, K-60, and K-60r, 10 pM; V-18r, 20 pM (r=active site revertant). Binding constants for each competitive inhibitor were first estimated by determining IC₅₀ values with 12 inhibitor concentrations and solving for an estimated Ki value using the equation Ki=IC₅₀×KM/(KM+[S]). The Ki value was then redetermined in separate assays using a series of inhibitor concentrations that equaled 0.5, 1, 2, and 3 times the estimated Ki value. Six substrate concentrations ranging from 50 to 600 μM were used for each inhibitor concentration. The final Ki values were derived from replots of KM/Vmax versus inhibitor concentration from double-reciprocal plots. The Ki values for each inhibitor with wild-type enzyme and selected others (e.g. the K-60 and saquinavir pair) were determined multiple times to yield an average S.D. of 4.2% (n=14). Other assay conditions were as described previously (Schock, H. et al (1996) J. Biol. Chem. 271, 31957-31963) with the exception that detection of product was monitored with fluorescence (excitation=270 nm, emission=330 nm).

In particular, the compounds of the following examples had activity in inhibiting HIV protease in the aforementioned assays, generally with an IC₅₀ from about 1 nM to 100 μM. Such a result is indicative of the intrinsic activity of the compounds in use as inhibitors of HIV protease activity.

Several methods for preparing the compounds of this invention are illustrated in the Schemes and Examples herein. Starting materials are made according to procedures known in the art or as illustrated herein. The following examples are provided so that the invention might be more fully understood. These examples are illustrative only and should not be construed as limiting the invention in any way.

Example 1 illustrates a synthesis according to Method A. Example 2 illustrates a synthesis according to Method B. Examples 3-5 and 7 illustrate syntheses according to Method C. Example 6 illustrates a synthesis according to Method D.

The following abbreviations are used throughout the text:

Me: methyl

Et: ethyl

Ar: aryl

Ph: phenyl

Ac: acetyl

DMF: N,N′-dimethyl formamide

THF: tetrahydrofuran

DMSO: dimethylsulfoxide

EDTA: ethylene diamine tetraacetic acid

Boc: tert-butyloxy carbonyl

BOP: Benzotriazol-1-yloxy-tris(dimethylamino)phosphonium hexafluorophosphate

BSA: bovine serum albumin

CHAPS: 3-[(3-cholamidopropyl)dimethylammonio]-2-hydroxy-1-propanesulfonate

TEA: triethylamine

TFA: trifluoroacetic acid

NIS: N-iodo succinimide

NaHMDS: sodium bis(trimethylsilyl)amide

DIPEA: diisopropylethylamine

DCM: dichloromethane

Nu: nucleophile

AIBN: 2,2′-azobisisobutyronitrile

MNNG: 1-methyl-3-nitro-1-nitrosoguanidine

rt: room temperature

HPLC: high performance liquid chromatography

LCMS: liquid chromatography mass spectrometry

### EXAMPLE 1

### (±)5,6,7,8-tetrahydro-4H-4,7-methanocyclohepta[d][1,3]-thiazol-2-amine

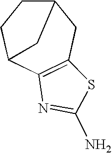

A mixture containing 124 mg (1.0 mmol) of bicyclo[3.2.1]octan-2-one, 253 mg (1.0 mmol) of iodine and 152 mg (2.0 mmol) of thiourea were heated at 110° C. in a sealed tube for 17 h. The dark reaction mixture was cooled and dissolved in 2 mL of methanol and subjected to reverse phase chromatography to yield the TFA salt of the desired aminothiazole as a white solid. 1H NMR (CD3OD) δ 8.65 (bs, 2H), 3.15 (t, 1H), 2.79 (dd, 1H), 2.64 (bt, 1H), 2.22 (d, 1H), 2.1-1.7 (m, 5H), 1.45 (dq, 1H). LCMS (M+H)=181.24

### EXAMPLE 2

### (±)6-phenyl-4,5,6,7-tetrahydro-1,3-benzothiazol-2-amine

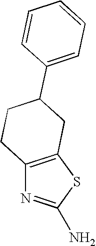

Step A: To a solution of 6.0 g (34.4 mrnmol) of 4-phenylcyclohexanone in 75 mL of CCl₄ was added 5.51 g (39.9 mmol) of N-bromosuccinimide and 83 mg (0.34 mmol) of AIBN. The mixture was stirred for 20 min at reflux before it was cooled and filtered. The filtrate was concentrated and subjected to column chromatography (9:1 Hexanes/EtOAc) to yield 2-bromo-4-phenylcyclohexanone.

Step B: A solution containing 6.0 g (23.7 mmol) of the bromo ketone from step A was treated with 1.8 g (23.7 mmol) of thiourea and the resulting mixture was stirred at ambient temperature over 48 h. The mixture was concentrated and triturated with ether to subjected to afford the desired compound as the HBr salt. 1H NMR (DMSO-d6) δ 9.21 (bs, 2H), 7.42-7.21 (m, 5H), 3.05 (m, 1H), 2.79 (m, 1H) 2.6-2.4 (m, 4H), 2.0-1.8 (m, 2H). LCMS (M+H)=231.23

### EXAMPLE 3

### 4-[1-(3-fluorophenyl)cyclopentyl]-1,3-thiazol-2-amine

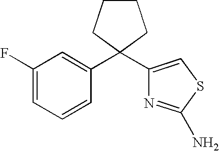

Step A: To a −70° C. solution containing 1.04 g (5.00 mmol) of 1-(3-fluorophenyl)-1-cyclopentane carboxylic acid in 25 mL of ether was added 530 mg (5.24 mmol) of N-methylmorpholine and 716 mg (5.24 mmol) of isobutyl chloroformate. The reaction mixture was stirred for 1 h then filtered through a fine frit funnel. The filtrate was cooled to 0° C. and excess CH₂N₂ (40 mL of diazomethane prepared from 50 mL ether/15 mL 40% KOH and 4.4 g MNNG) was pipetted into the flask containing the mixed anhydride. The resulting mixture was stirred until LCMS showed complete conversion to the diazoketone (14 h) then the excess CH₂N₂ was evaporated. The resulting yellow oil was dissolved in ether and cooled to 0° C. and treated with 48% HBr (1.5 mL). Effervescence occurred within 10 seconds and LCMS detected complete conversion to a new peak after 1 h. The reaction mixture was diluted with 50 mL of ether and washed with saturated bicarbonate 2×10 mL, water (10 mL) and brine (10 mL). Evaporation of the solvent left the bromo ketone which was used without further purification.

Step B: A stirred mixture containing 1.3 g (4.56 mmol) of the bromo ketone from step 3-A and 383 mg (4.56 mmol) of NaHCO₃, and 347 mg (4.56 mmol) of thiourea in 25 mL of EtOH was heated at reflux for 1 h. The reaction mixture was cooled, concentrated and subjected to reverse phase chromatography to afford the TFA salt of the desired compound as a white solid. 1H NMR (CD3OD) δ 7.38 (q, 1H), 7.17 (d, 1H), 7.09 (d, 1H), 7.01 (t, 1H), 6.76 (s, 1H), 2.4-2.2 (m, 4H). LCMS (M+H)=263.1

### EXAMPLE 4

### N′-{5-[2-(amino-1,3-thiazol-4-yl)-2-(4-methoxyphenyl)ethyl]pyridin-2-yl}, N-N-dimethylethane-1,2-diamine

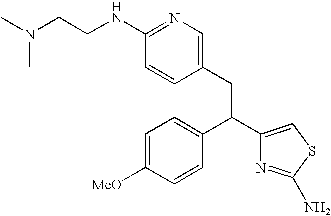

Step A: NaHMDS (8.0 mL, 8.0 mmol) was added to a −70° C. solution of ethyl 4-methoxyphenyl acetate (1.55 g, 8.0 mmol) and 2-chloro-5-chloromethylpyridine (1.29 g, 8.0 mmol) in 25 mL of THF. The reaction mixture was stirred to rt over a period of 16 h after which time the solvent was evaporated and the residue partitioned between 20 mL of EtOAc and 20 mL of saturated ammonium chloride. The aqueous phase was washed 2×25 mL of EtOAc and the combined organic extracts were washed with brine (20 mL) and dried over MgSO₄. Evaporation of the solvent left the monoalkylated target as a colorless oil.

Step B: To a solution containing 851 mg (2.66 mmol) of the ester from step 4-A in 20 mL of dioxane was added 8 mL (8 mmol) of a 1M LiOH. The reaction was allowed to stir over 16 h and the solvent was evaporated and the residue was treated with 3N HCl to pH=6. The aqueous mixture was extracted with EtOAc (3×20 mL) and the combined organic washings were dried over MgSO₄ and evaporated to afford the desired carboxylic acid.

Step C: To a −70° C. solution containing 760 mg (2.61 mmol) of the carboxylic acid from step 4-B in 15 mL of ether was added 0.30 mL (2.74 mmol) of N-methylmorpholine and 374 mg (2.74 mmol) of isobutyl chloroformate. The reaction mixture was stirred for 15 min then quenched with 5 mL of water. The phases were separated and the ether layer was dried and evaporated. Excess CH₂N₂ (40 mL of diazomethane prepared from 50 mL ether/15 mL 40% KOH and 4.4 gram MNNG) was pipetted into the flask containing the mixed anhydride at rt and the resulting mixture was stirred until LCMS showed complete conversion to the diazoketone (30 min to 24 h). Excess CH₂N₂ was evaporated and the resulting yellow oil was redissolved in ether and cooled to 0° C. and treated with 48% HBr (1.5 mL). Effervescence occurred within 10 seconds and LCMS detected complete conversion to a new peak after 1 h. The reaction mixture was diluted with 50 mL of ether and washed with saturated bicarbonate 2×10 mL, water (10 mL) and brine (10 mL). Evaporation of the solvent left the bromo ketone as a white solid that was used without further purification.

Step D: A solution containing 790 mg (2.14 mmol) of the bromo ketone from step 4-C in 10 mL of MeOH was treated with 180 mg (2.14 mmol) of NaHCO₃ and 163 mg (2.14 mmol) of thiourea and heated at 50° C. for 1 h. The mixture was then concentrated and extracted with water and EtOAc. The organic phase was dried, concentrated and chromatographed (EtOAc) to afford the desired 2-aminothiazole as an off-white solid. 1H NMR (CDCl3) δ 8.05 (d, J=2.2 Hz, 1H), 7.24 (dd, J=2.4, 8.2 Hz, 1H), 7.14 (m, 2H), 6.80 (d, J=8.6 Hz, 1H), 6.06 (s, 1H), 4.80 (s, 2H), 4.00 (t, J=7.5 Hz, 1H), 3.77 (s, 3H), 3.45 (dd, J=7.0, 13.7 Hz, 1H), 3.08 (dd, J=8.6, 13.7 Hz, 1H). LCMS (M+H)=346.03.

Step E: A neat mixture containing 56 mg (0. 16 mmol) of the chloropyridine from step 4-D and 301 mg (3.40 mmol) of N,N-dimethylehylenediamine was heated at 140° C. in a sealed tube for 17 h. The reaction was cooled and dissolved in 2 mL of methanol and subjected to reverse phase chromatography. The solvents were evaporated and the residue was dissolved in methanol and treated with gaseous HCl. Evaporation of the solvent left the tris HCl salt of the desired aminothiazole as a tan colored solid. 1H NMR (CD3OD) δ 8.90 (d, J=8.9 Hz, 1H), 7.61 (s, 1H), 7.12 (d, J=8.7 Hz, 2H), 7.01 (d, J=9.2 Hz, 1H), 6.89 (d, J=8.9 Hz, 2H), 6.71 (s, 1H), 4.15 (t, J=7.5 Hz, 1H), 3.77 (m, 2H), 3.75 (s, 3H), 3.41 (m, 3H), 3.28 (s, 3H), 3.05 (m, 1H), 2.97 (s, 3H). LCMS (M+H)=398.12.

### EXAMPLE 5

### 4-[(1S)-2-(4-iodophenyl)-1-(4-methoxyphenyl)ethyl]-1,3-thiazol-2-amine

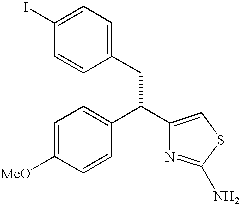

Step A. (S)-4-benzyl-2-oxazolidinone (8.00 g, 45.1 mmol) and p-methoxyphenylacetic acid (15.0 g, 90.3 mmol) were dissolved in 90 mL of toluene and treated with 18.2 g (180.5 mmol) of TEA. Pivaloyl chloride (10.9 g, 90.2 mmol) in 50 mL of toluene was added dropwise and the resulting solution was heated at reflux for 17 h. The reaction mixture was cooled and the organic phase was washed with 1N HCl (2×50 mL), water, saturated NaHCO₃ (2×50 mL), and brine. After drying (MgSO₄), the solution was concentrated and chromatographed (20% to 30% EtOAc/Hexanes) to provide the desired compound. 1H NMR (CDCl3) δ 7.4-7.2 (m, 5H), 7.17 (d, J=7.8 Hz, 2H), 6.84 (d, J=7.8 Hz, 2H), 4.64 (m, 1H), 4.3-4.1 (m, 2H), 3.81 (s, 3H), 3.22 (dd, J=3.1, 13.3 Hz, 1H), 2.65 (dd, J=9.5, 13.6 Hz, 1H). LCMS (M+H)=326.14

Step B. NaHMDS (40.5 mL, 40.5 mmol) was added to a −70° C. solution of the oxazolidinones from step 5-A (10.99 g, 33.77 mmol) and 4-iodobenzyl bromide (20.0 g, 67.5 mmol) in 100 mL of THF. The reaction mixture was stirred at this temperature for 5 h then quenched with 90 mL of saturated NH₄Cl solution. The mixture was extracted with EtOAc×3 and the combined organics were washed with 20 mL of brine. Evaporation and chromatography (10% to 30% EtOAc/Hexanes) left the desired compound as a single diastereomer. 1H NMR (CDCl3) δ 7.60 (d, J=7.8 Hz, 2H), 7.33 (d, J=7.8 Hz, 2H), 7.25 (m, 4H), 6.99 (m, 3H), 6.84 (d, J=7.8 Hz, 2H), 5.32 (dd, J=6.2, 9.3 Hz, 1H), 4.58 (m, 1H), 4.11 (m, 2H), 3.76 (s, 3H), 3.42 (dd, J=9.4, 13.6 Hz, 1H), 3.08 (dd, J=3.1, 13.3 Hz, 1H) 2.96 (dd, J=6.2, 13.5 Hz, 1H), 2.60 (dd, J=9.0, 13.6 Hz, 1H). LCMS (M+H)=542.20

Step C. The oxazolidinones from step 5-B (514 mg, 0.949 mmol) in 3:1 THF/water (8 mL) was cooled to 0° C. and treated with 45 mg LiOH monohydrate dissolved in 1.5 mL of water then 0.38 mL of hydrogen peroxide. The mixture was stirred for 45 min then quenched with 20 mL of saturated Na₂SO₃. The reaction mixture was extracted 3×25 mL of dichloromethane and the combined organic extracts were discarded. The aqueous phase was acidified with 4 mL of 1N HCl, washed with DCM×5 then dried over MgSO₄. Evaporation of the solvent left the desired carboxylic acid. 1H NMR (CDCl3) δ 7.58 (d, J=7.8 Hz, 2H), 7.33 (d, J=7.8 Hz, 2H), 7.25 (d, J=7.6 Hz, 4H), 6.84 (d, J=7.8 Hz, 2H), 3.76 (s, 3H), 3.75 (m, 1H), 3.24 (dd, J=8.2, 13.9 Hz, 1H), 2.91 (dd, J=7.3, 13.9 Hz, 1H).

Step D. To a 0° C. solution containing 253 mg (0.66 mmol) of the carboxylic acid from step 5-C in 3 mL of THF was added 70 mg (0.69 mmol) of N-methylmorpholine and 95 mg (0.69 mmol) of isobutyl chloroformate. The reaction mixture was stirred for 15 min then the solid NMM salt was filtered off and the filtrate was evaporated. Excess CH₂N₂ (prepared from 11 mL ether/3.5 mL 40% KOH and 976 mg MNNG) was pipetted into the flask containing the mixed anhydride at rt and the resulting mixture was stirred until LCMS showed complete conversion to the diazoketone (16 h). Excess CH₂N₂ was evaporated and the resulting yellow oil was redissolved in ether and cooled to 0° C. and treated with 48% HBr (107 mg). Effervescence occurred within 10 seconds and LCMS detected complete conversion to a new peak after 1 h. The reaction mixture was diluted with 10 mL of ether and washed with saturated bicarbonate 2×3 mL, water (3 mL) and brine (3 mL). Evaporation of the solvent left the bromo ketone as a an oil that was used without further purification.

Step E. A solution containing 307 mg (0.669 mmol) of the bromo ketone from step 5-D in 3 mL of MeOH was treated with 56 mg (0.669 mmol) of NaHCO₃ and 51 mg (0.669 mmol) of thiourea and heated at 50° C. for 15 min. The mixture was then concentrated and extracted with water and EtOAc. The organic phase was dried, concentrated and chromatographed (reverse phase LC) to afford the TFA salt of the desired 2-aminothiazole as an off-white solid. 1H NMR (CD3OD) δ 7.48 (d, J=7.8 Hz, 2H), 7.11 (d, J=7.8 Hz, 2H), 6.88 (d, J=7.6 Hz, 4H), 6.15 (s, 1H), 3.99 (t, J=8.5 Hz, 1H), 3.70 (s, 3H), 3.74 (m, 1H), 3.03 (dd, J=7.3, 13.9 Hz, 1H). LCMS (M+H)=437.1

### EXAMPLE 6

### 4-[1-(4-chlorophenyl)cyclopentyl]-5-phenyl-1,3-thiazol-2-amine

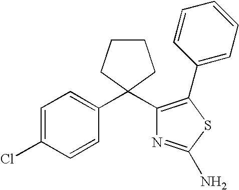

Step A: To a 0° C. solution containing 486 mg (2.0 mmol) of 1-(4-chlorophenyl)-1-cyclopentanecarbonyl chloride and 196 mg (2.0 mmol) of N,O-dimethylhydroxylamine HCL in 20 mL of DCM was added 1.4 mL (10.0 mmol) of TEA. The reaction mixture was stirred to rt over 16 h then washed with water (2×5 mL), 1N HCl (2×5 mL), and brine. The dried organic extract was chromatographed (1: Hexanes/EtOAc) to yield the desired amide.

Step B: To a 0° C. solution of 358 mg (1.38 mmol) of the Weinreb amide from step 6-A in 10 mL of THF was added 1.4 mL (2.8 mmol) of benzyl magnesium bromide. The reaction was allowed to stir to rt over 17 h before it was diluted with 20 mL of ether and quenched with 5 mL of saturated ammonium chloride. The organic phase was isolated and washed with brine. Column chromatography (4:1 Hexanes/EtOAc) left the desired ketone which was used in the next step. LCMS (M+H)=299.10.

Step C: A solution containing 320 mg (1.0 mmol) of the ketone from step 6-B in 10 mL of chloroform was treated with 171 mg (1.0 mmol) of bromine and heated at 50° C. for 30 min. The reaction mixture was cooled and washed with saturated bicarbonate solution (2×5 mL), water, then brine. The organic phase was dried over MgSO₄ and evaporated to leave the desired α-bromo ketone which was used without further purification.

Step D: A solution containing 377 mg (1.0 mmol) of the bromo ketone from step 6-C, 84 mg (1.0 mmol) of NaHCO₃, and 76 mg (1.0 mmol) of thiourea in 10 mL of methanol was heated at 50° C. for 16 h. The reaction was cooled and concentrated to ¼ volume and chromatographed using reverse phase LC to afford the desired inhibitor as the mono TFA salt. 1H NMR (CDC₁₃ δ 9.02 (bs, 2H), 7.42-7.17 (m, 9H), 2.22 (m, 1H), 2.01 (m, 1H), 1.65 (m, 1H), 1.45 (m, 1H). LCMS (M+H)=355.01.

### EXAMPLE 7

### 5-(2-furyl)-4-[2-(2-methoxy-5-nitrophenyl)-1-(4-methoxyphenyl)ethyl-1,3-thiazol-2-amine

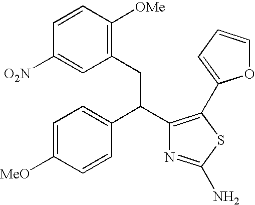

Step A: NaHMDS (30.0 mL, 30.0 mmol) was added to a −70° C. solution of ethyl 4-methoxyphenyl acetate (5.83 g, 30.0 mmol) and 2-methoxy-5-nitrobenzyl bromide (7.38 g, 30.0 mmol) in 200 mL of THF. The reaction mixture was stirred to rt over a period of 16 h after which time the solvent was evaporated and the residue partitioned between 150 mL of EtOAc and 20 mL of saturated ammonium chloride. The aqueous phase was washed 2×25 mL of EtOAc and the combined organic extracts were washed with brine (20 mL) and dried over MgSO₄. The organic phase was dried, concentrated and chromatographed (0-50% EtOAc/hexane) to afford the monoalkylated target.

Step B: To a solution containing 6.67 g (18.6 mmol) of the ester from step 1 in 100 mL of methanol and 100 mL of THF was added 37 mL (37 mmol) of a 1M LiOH. The reaction was allowed to stir over 16 h and the solvent was evaporated and the residue was treated with 3N HCl to pH=6. The aqueous mixture was extracted with EtOAc (3×20 mL) and the combined organic washings were dried over MgSO₄ and evaporated to afford the desired carboxylic acid.

Step C: To a −70° C. solution containing 2.5 g (7.54 mmol) of the carboxylic acid from step B in 100 mL of ether was added 0.87 mL (7.92 mmol) of N-methylmorpholine and 1.08 g (7.92 mmol) of isobutyl chloroformate. The reaction mixture was stirred for 15 min then filtered through a fine fritted funnel. Excess CH₂N₂ (75 mL of diazomethane prepared from 75 mL ether/23 mL 40% KOH and 6.64 gram MNNG) was pipetted into the flask containing the mixed anhydride at rt and the resulting mixture was stirred until LCMS showed complete conversion to the diazoketone (30 min to 24 h). Excess CH₂N₂ was evaporated and the resulting yellow oil was redissolved in ether and cooled to 0° C. and treated with 48% HBr (2.0 mL). Effervescence occurred within 10 seconds and LCMS detected complete conversion to a new peak after 1 h. The reaction mixture was diluted with 50 mL of ether and washed with saturated bicarbonate 2×10 mL, water (10 mL) and brine (10 mL). Evaporation of the solvent left the bromo ketone as a white solid that was used without further purification.

Step D: A solution containing 3.1 g (7.54 mmol) of the bromo ketone from step C in 10 mL of MeOH was treated with 634 mg (7.54 mmol) of NaHCO₃ and 574 mg (7.54 mmol) of thiourea and heated at 50° C. for 1 h. The mixture was then concentrated and extracted with water and CH₂Cl₂. The organic phase was dried, concentrated and chromatographed (EtOAc) to afford the desired aminothiazole. LCMS (M+H)=386.0.

Step E: To a 0° C. solution containing 710 mg (1.84 mmol) of the aminothiazole from step D in 3 mL of CHCl₃ was added 608 mg (2.39 mmol) iodine. The solution warmed to rt and stirred for 16 h. The solution was diluted with 10 mL of CH₂Cl₂ and washed with saturated NaHCO₃. The organic layer was concentrated and chromatographed (20-100% EtOAc/hexane) to give the 5-iodinated aminothiazole. LCMS (M+H)=511.9.

Step F: To a solution containing 59.8 mg (0.12 mmol) of the 5-iodinated aminothiazole from step E in 2 mL of DMF was added 41.8 mg (0.12 mmol) tributyl(2-furyl)tin. The solution was degassed and 4.1 mg (0.01 mmol) bis(triphenylphosphine) palladium(II) chloride was added. The solution was heated at 90° C. for 16 h. The solution was cooled and chromatographed (RPLC) to give the desired aminothiazole. ¹H NMR (CD₃OD) δ 8.01 (m, 1H), 7.89 (d, J=2.74 Hz, 1H), 7.45 (s, 1H), 7.34 (d, J=8.70 Hz, 2H), 7.00 (m, 1H), 6.95 (m, 2H), 6.45 (m, 1H), 6.31 (d, J=3.4 Hz, 1H), 3.86 (s, 3H), 3.81 (s, 3H), 3.47 (m, 1H), 3.35 (m, 1H). LCMS (M+H)=451.97

The compounds of the following examples were prepared in an analogous manner to that described in the Examples above, using methods A-D as described above.

| Example | Structure | Method | M + H |
|---|---|---|---|
| 8 | 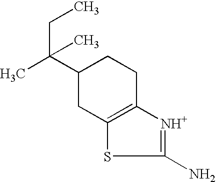 | A | 225.38 |
| 9 | 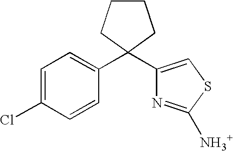 | C | 279.8 |
| 10 | 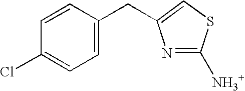 | C | 225.02 |
| 11 | 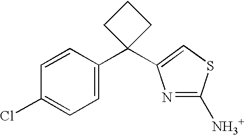 | C | 265.05 |
| 12 | 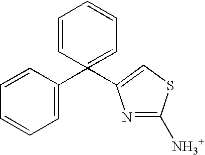 | C | 267.36 |
| 13 | 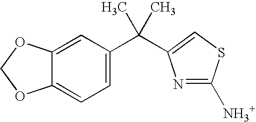 | C | 263.08 |
| 14 | 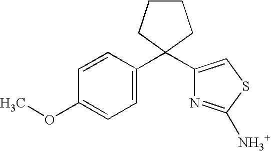 | C | 275.11 |
| 15 | 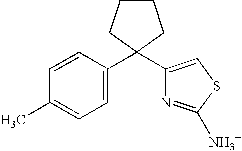 | C | 259.12 |
| 16 | 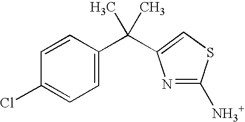 | C | 253.76 |
| 17 | 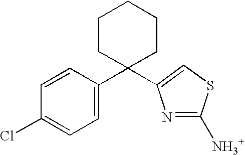 | C | 293.08 |
| 18 | 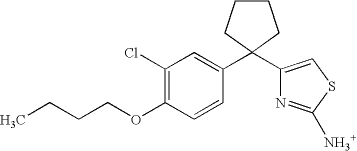 | C | 351.12 |
| 19 | 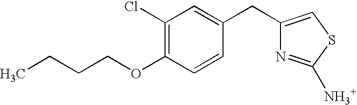 | C | 297.08 |
| 20 | 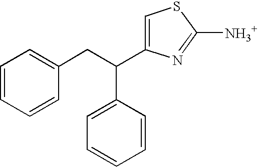 | C | 281.39 |
| 21 | 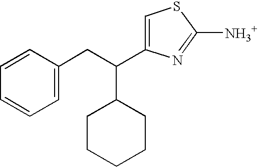 | C | 273.41 |
| 22 | 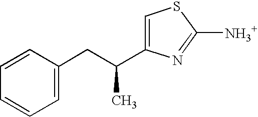 | C | 205.29 |
| 23 | 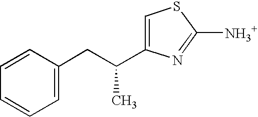 | C | 205.29 |
| 24 | 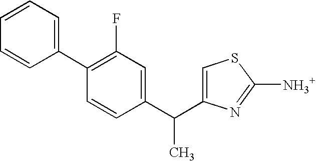 | C | 299.38 |
| 25 | 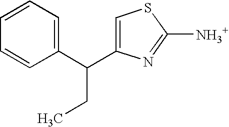 | C | 219.32 |
| 26 | 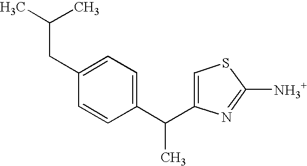 | C | 261.4 |
| 27 | 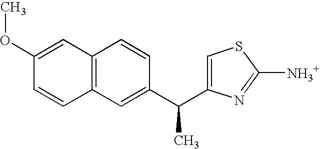 | C | 285.38 |
| 28 | 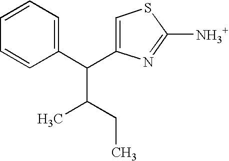 | C | 247.37 |
| 29 | 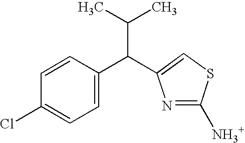 | C | 267.79 |
| 30 | 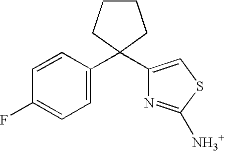 | C | 263.09 |
| 31 | 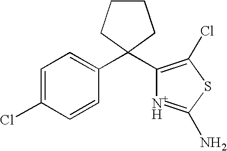 | C | 313.03 |
| 32 | 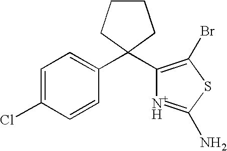 | C | 356.97 |
| 33 | 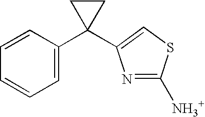 | C | 217.07 |
| 34 | 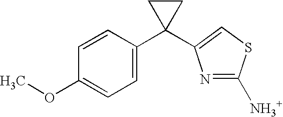 | C | 247.08 |
| 35 | 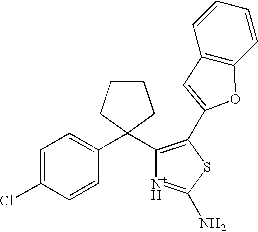 | C | 395.09 |
| 36 | 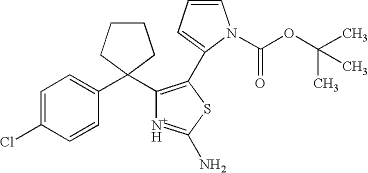 | C | 444.14 |
| 37 | 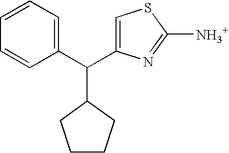 | C | 259.39 |
| 38 | 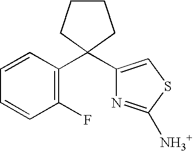 | C | 263.09 |
| 39 | 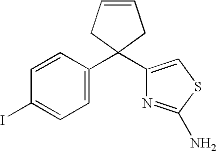 | C | 369 |
| 40 | 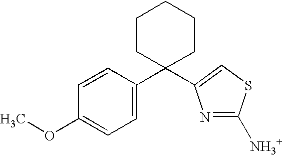 | C | 289.13 |
| 41 | 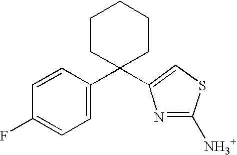 | C | 277.11 |
| 42 | 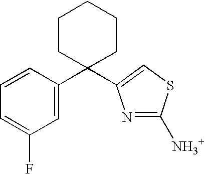 | C | 277.11 |
| 43 | 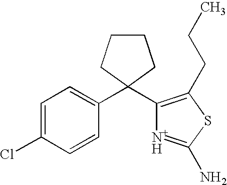 | D | 321.11 |
| 44 | 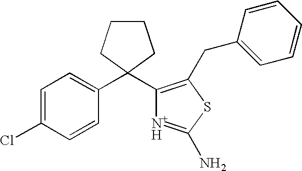 | D | 369.11 |
| 45 | 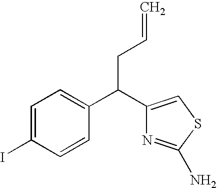 | C | 357.23 |
| 46 | 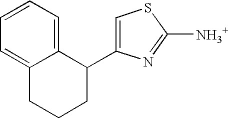 | A | 231.33 |
| 47 | 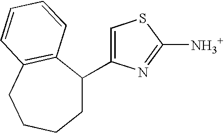 | A | 245.36 |
| 48 | 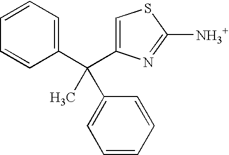 | C | 281.1 |
| 49 | 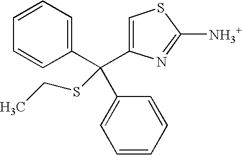 | C | 327.09 |
| 50 | 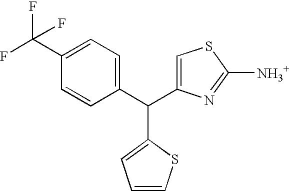 | C | 341.36 |
| 51 | 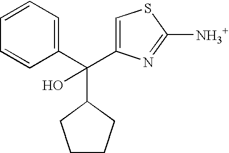 | C | 275.13 |
| 52 | 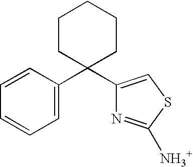 | C | 259.12 |
| 53 | 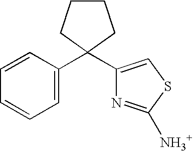 | C | 245.1 |
| 54 | 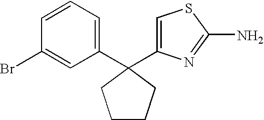 | C | 323.01 |
| 55 | 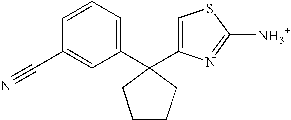 | C | 270.1 |
| 56 | 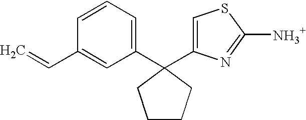 | C | 271.12 |
| 57 | 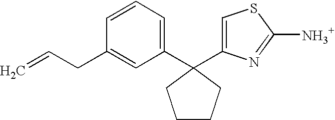 | C | 385.13 |
| 58 | 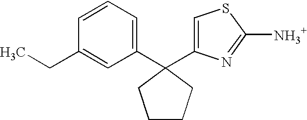 | C | 273.12 |
| 59 | 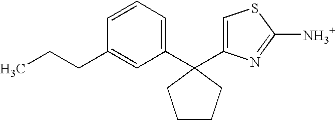 | C | 287.15 |
| 60 | 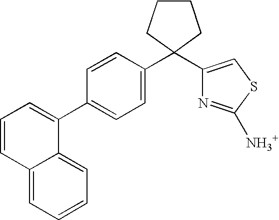 | C | 371.15 |
| 61 | 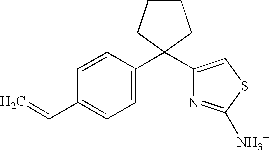 | C | 271.12 |
| 62 | 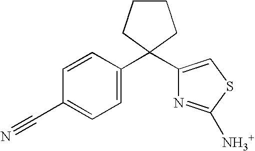 | C | 270.1 |
| 63 | 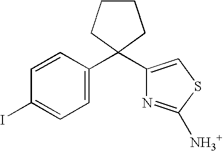 | C | 371 |
| 64 | 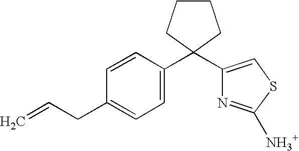 | C | 385.13 |
| 65 | 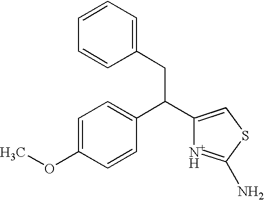 | C | 311.42 |
| 66 | 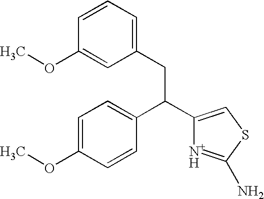 | C | 341.44 |
| 67 | 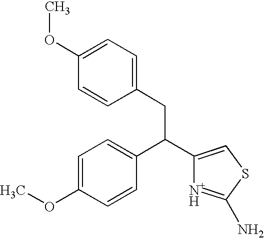 | C | 341.44 |
| 68 | 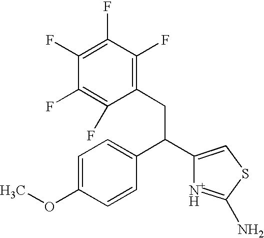 | C | 401.37 |
| 69 | 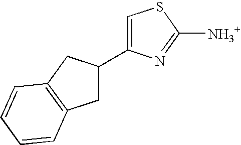 | C | 217.07 |
| 70 | 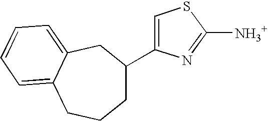 | C | 245.34 |
| 71 | 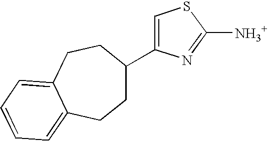 | C | 245.34 |
| 72 | 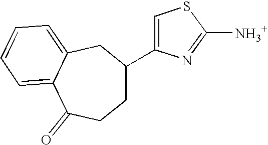 | C | 259.31 |
| 73 | 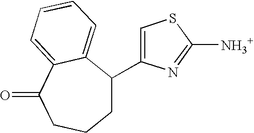 | C | 259.32 |
| 74 | 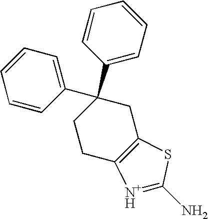 | A | 321.12 |
| 75 | 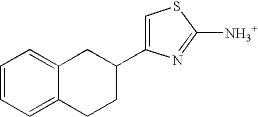 | C | 231.09 |
| 76 | 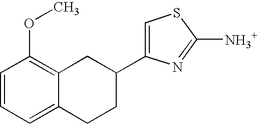 | C | 261.1 |
| 77 | 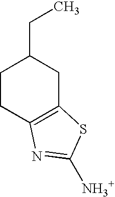 | A | 183.11 |
| 78 | 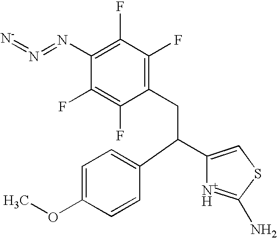 | C | 424.39 |
| 79 | 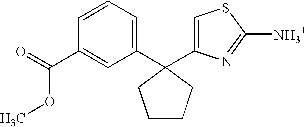 | C | 303.11 |
| 80 | 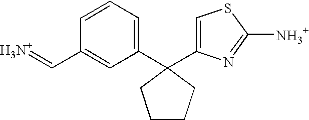 | C | 274.13 |
| 81 | 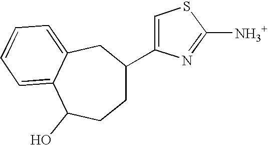 | C | 261.36 |
| 82 | 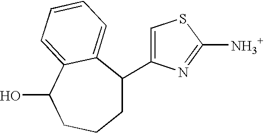 | C | 261.37 |
| 83 | 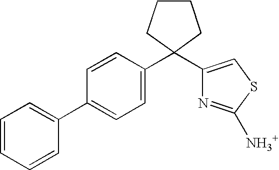 | C | 321.13 |
| 84 | 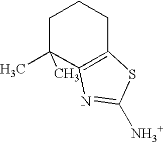 | A | 183.09 |
| 85 | 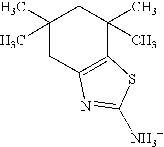 | A | 211.12 |
| 86 | 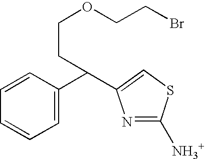 | C | 342.27 |
| 87 | 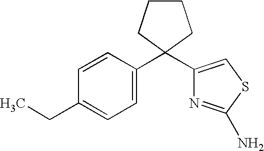 | C | 273.12 |
| 88 | 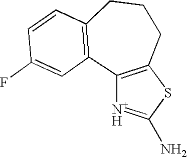 | C | 235.06 |
| 89 | 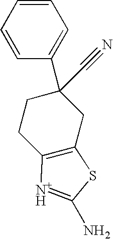 | A | 256.08 |
| 90 | 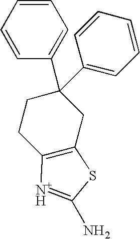 | A | 307.11 |
| 91 | 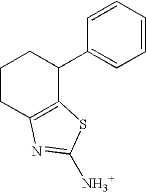 | A | 231.09 |
| 92 | 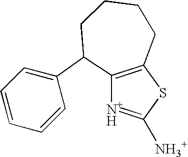 | B | 245.36 |
| 93 | 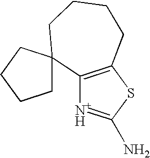 | A | 223.12 |
| 94 | 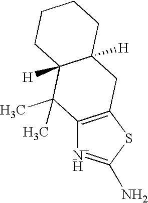 | B | 237.13 |
| 95 | 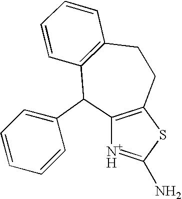 | A | 293.41 |
| 96 | 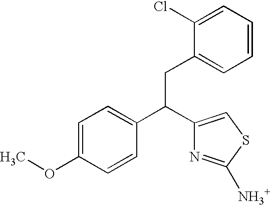 | C | 345.86 |
| 97 | 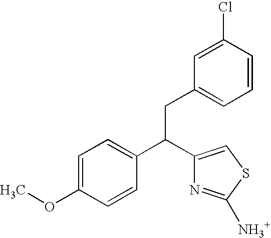 | C | 345.86 |
| 98 | 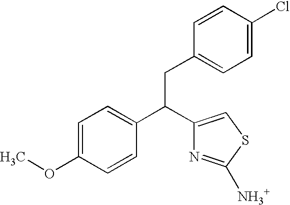 | C | 245.86 |
| 99 | 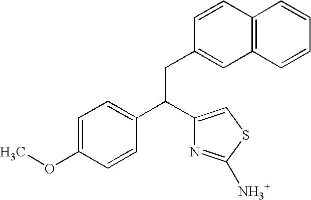 | C | 361.48 |
| 100 | 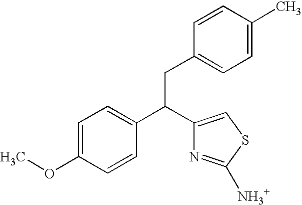 | C | 325.45 |
| 101 | 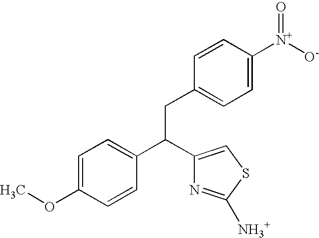 | C | 356..42 |
| 102 | 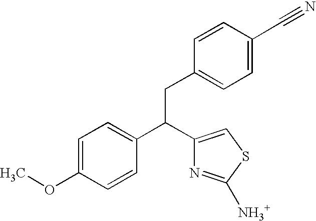 | C | 336.43 |
| 103 | 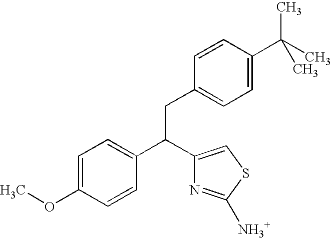 | C | 367.53 |
| 104 | 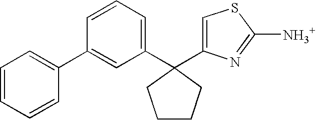 | C | 321.13 |
| 105 | 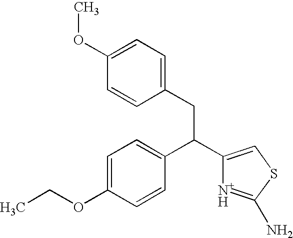 | C | 355.47 |
| 106 | 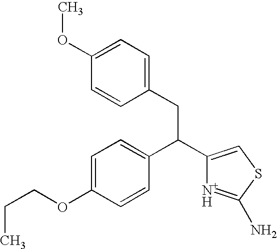 | C | 368.5 |
| 107 | 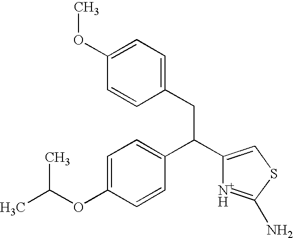 | C | 369.5 |
| 108 | 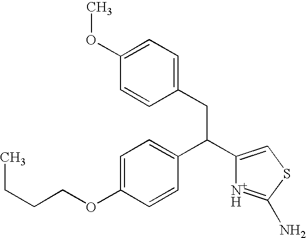 | C | 383.53 |
| 109 | 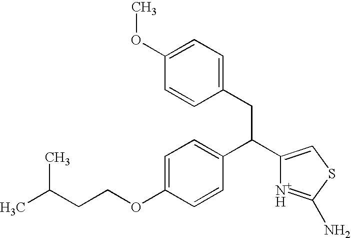 | C | 397.55 |
| 110 | 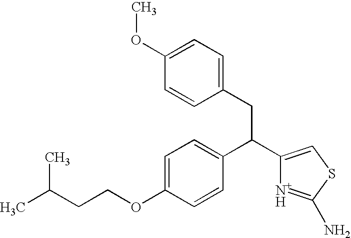 | C | 397.55 |
| 111 | 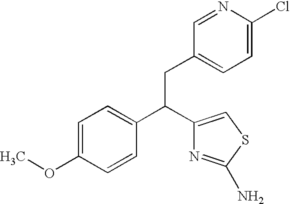 | C | 346.85 |
| 112 | 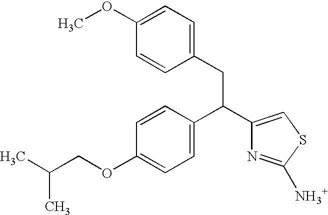 | C | 385.53 |
| 113 | 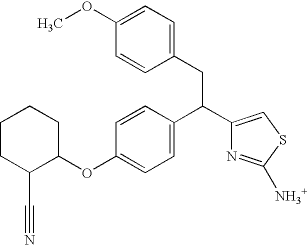 | C | 428.53 |
| 114 | 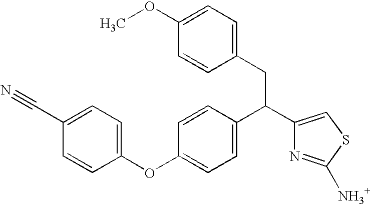 | C | 428.53 |
| 115 | 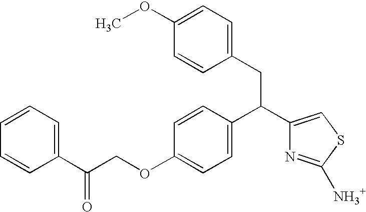 | C | 445.55 |
| 116 | 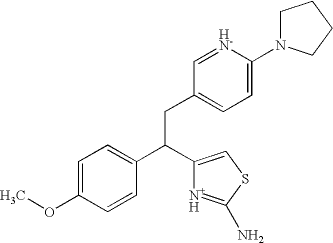 | C | 381.51 |
| 117 | 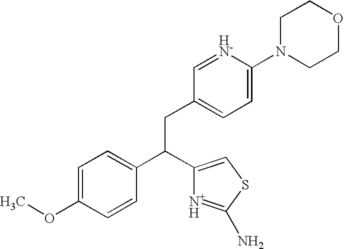 | C | 397.55 |
| 118 | 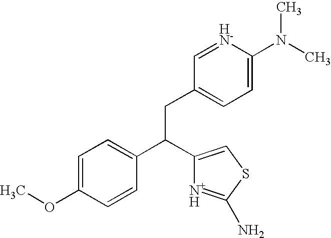 | C | 355.47 |
| 119 | 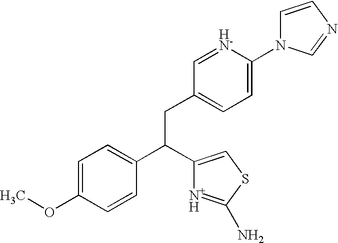 | C | 378.47 |
| 120 | 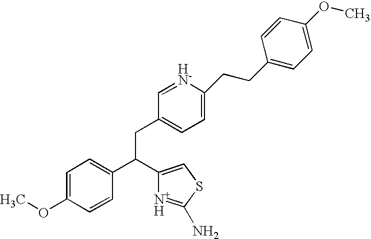 | C | 447.57 |
| 121 | 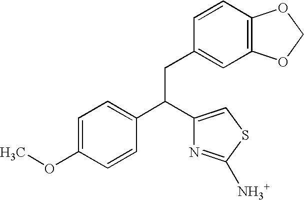 | C | 355.43 |
| 122 | 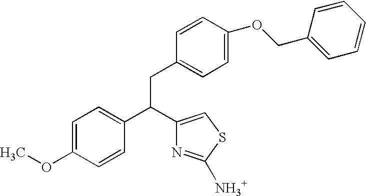 | C | 417.55 |
| 123 | 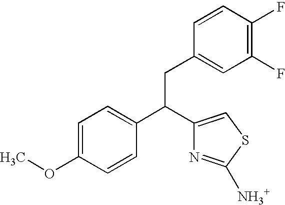 | C | 347.4 |
| 124 | 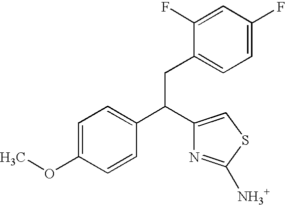 | C | 347.41 |
| 125 | 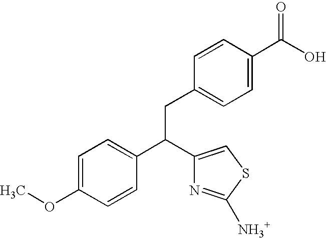 | C | 355.44 |
| 126 | 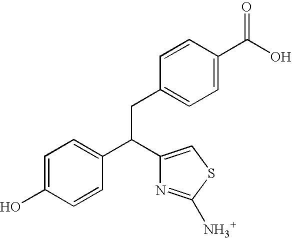 | C | 341.4 |
| 127 | 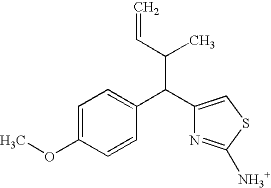 | C | 275.38 |
| 128 | 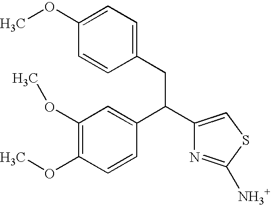 | C | 371.47 |
| 129 | 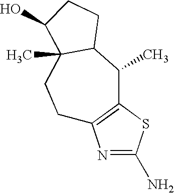 | A | 253.13 |
| 130 | 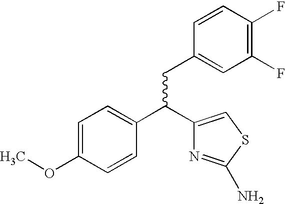 | C | 247.4 |
| 131 | 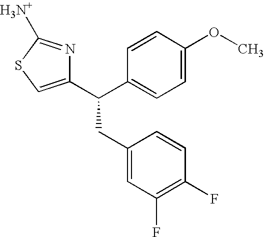 | C | 247.42 |
| 132 | 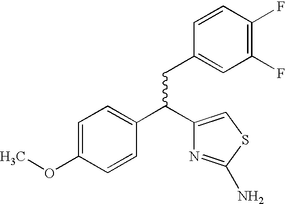 | C | 347.38 |
| 133 | 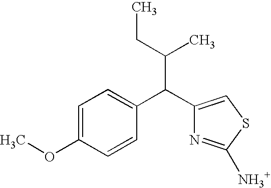 | C | 277.4 |
| 134 | 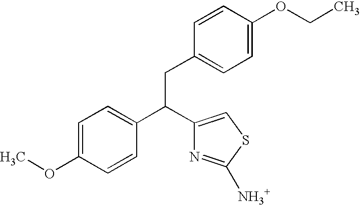 | C | 355.47 |
| 135 | 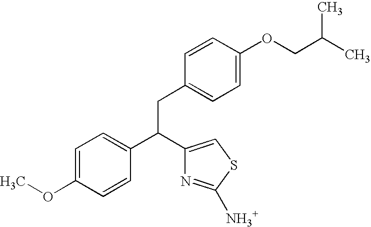 | C | 383.53 |
| 136 | 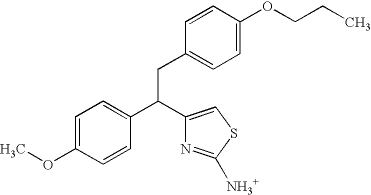 | C | 369.5 |
| 137 | 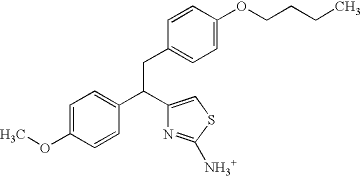 | C | 383.53 |
| 138 | 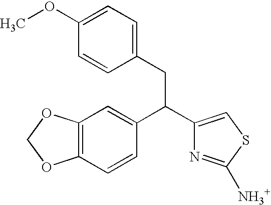 | C | 355.43 |
| 139 | 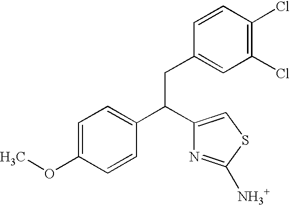 | C | 380.31 |
| 140 | 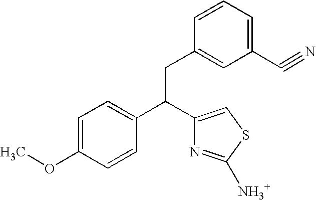 | C | 336.43 |
| 141 | 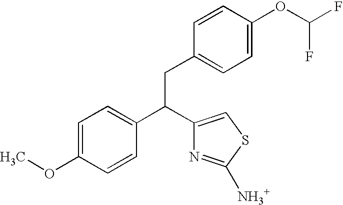 | C | 377.42 |
| 142 | 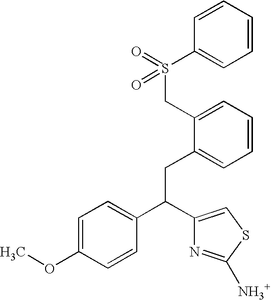 | C | 465.61 |
| 143 | 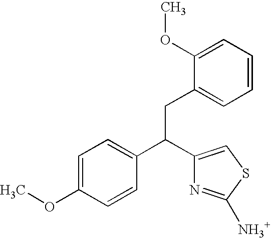 | C | 341.45 |
| 144 | 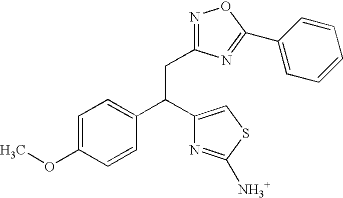 | C | 379.45 |
| 145 | 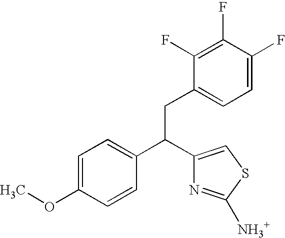 | C | 365.39 |
| 146 | 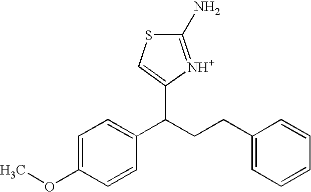 | C | 325.44 |
| 147 | 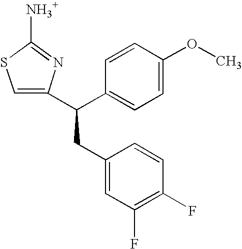 | C | 347.4 |
| 148 | 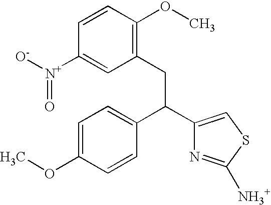 | C | 386.44 |
| 149 | 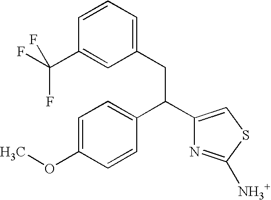 | C | 379.42 |
| 150 | 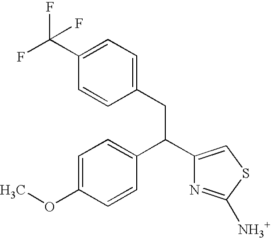 | C | 379.42 |
| 151 | 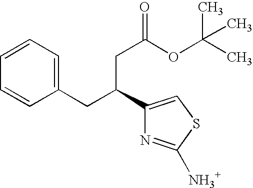 | C | 305.12 |
| 152 | 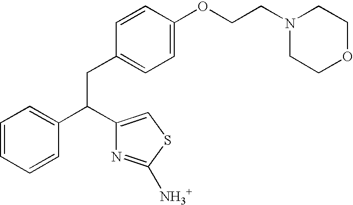 | C | 410.55 |
| 153 | 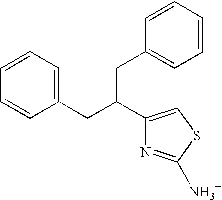 | C | 295.12 |
| 154 | 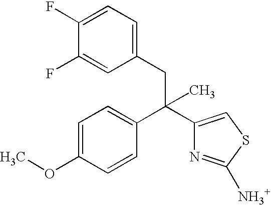 | C | 361.11 |
| 155 | 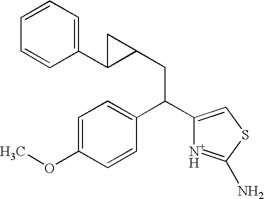 | C | 351.49 |
| 156 | 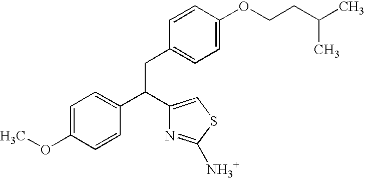 | C | 397.55 |
| 157 | 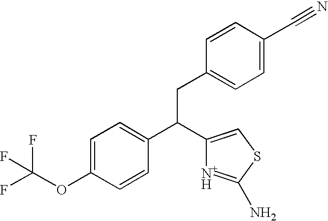 | C | 390.4 |
| 158 | 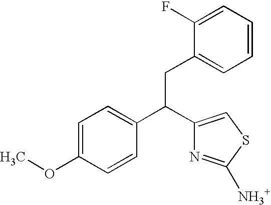 | C | 329.41 |
| 159 | 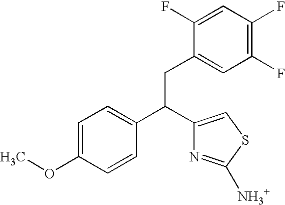 | C | 365.39 |
| 160 | 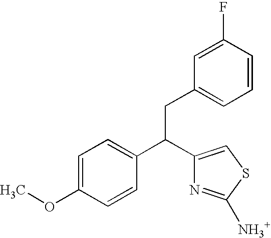 | C | 329.41 |
| 161 | 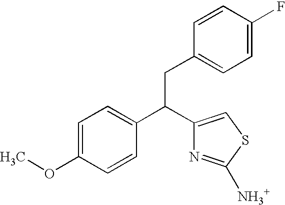 | C | 329.41 |
| 162 | 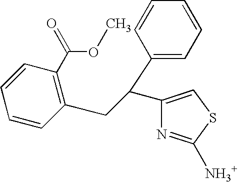 | C | 339.43 |
| 163 | 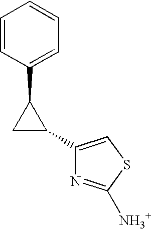 | C | 217.07 |
| 164 | 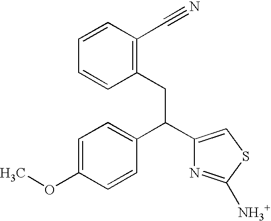 | C | 336.43 |
| 165 | 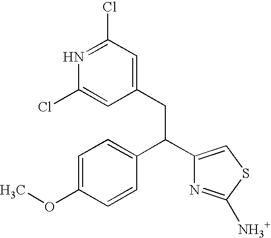 | C | 381.3 |
| 166 | 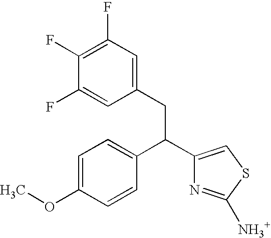 | C | 365.39 |
| 167 | 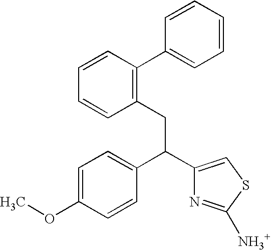 | C | 387.52 |
| 168 | 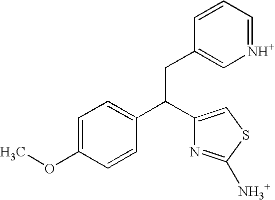 | C | 312.41 |
| 169 | 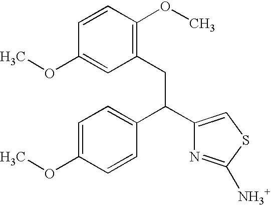 | C | 371.47 |
| 170 | 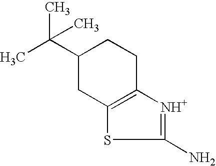 | A | 337.46 |
| 171 | 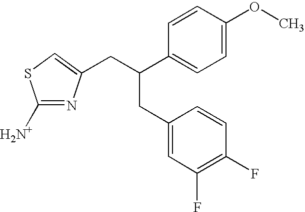 | C | 361.1 |
| 172 | 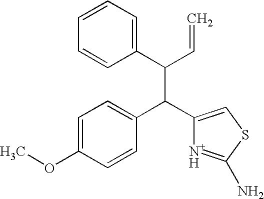 | C | 337.46 |
| 173 | 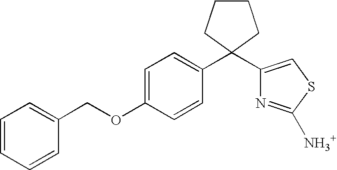 | C | 351.15 |
| 174 | 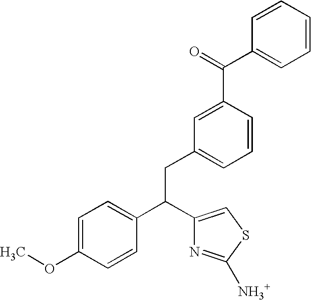 | C | 415.53 |
| 175 | 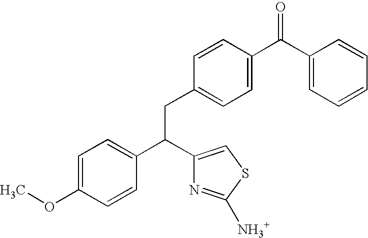 | C | 415.53 |
| 176 | 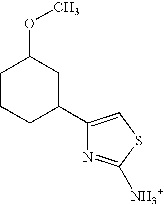 | A | 213.1 |
| 177 | 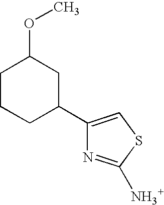 | A | 213.1 |
| 178 | 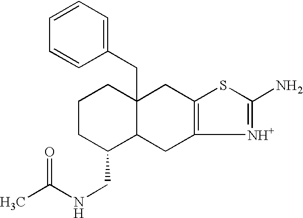 | B | 370.19 |
| 179 | 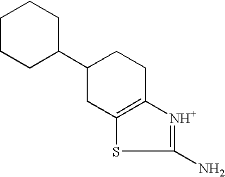 | A | 237.13 |
| 180 | 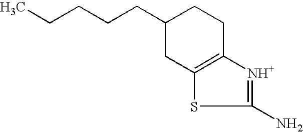 | A | 225.13 |
| 181 | 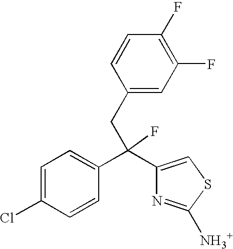 | C | 365.09 |
| 182 | 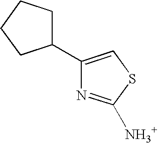 | A | 169.07 |
| 183 | 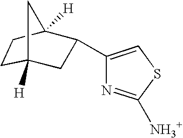 | A | 195.09 |
| 184 | 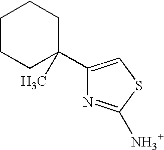 | A | 197.1 |
| 185 | 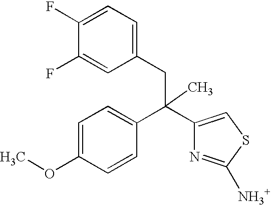 | C | 361.11 |
| 186 | 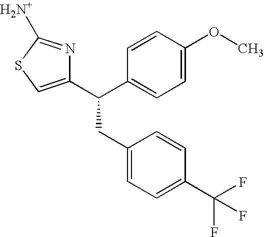 | C | 379.41 |
| 187 | 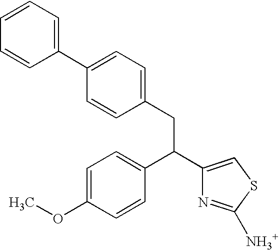 | C | 387.52 |
| 188 | 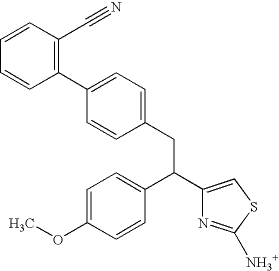 | C | 412.53 |
| 189 | 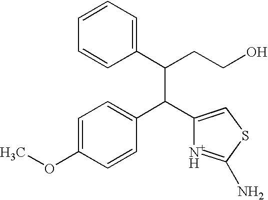 | C | 355.47 |
| 190 | 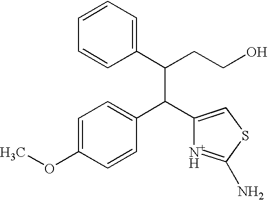 | C | 355.47 |
| 191 | 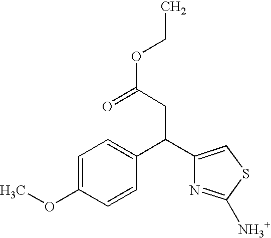 | C | 307.38 |
| 192 | 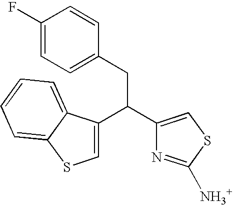 | C | 355.07 |
| 193 | 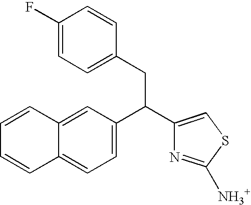 | C | 349.44 |
| 194 | 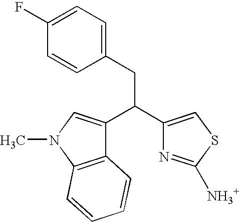 | C | 352.12 |
| 195 | 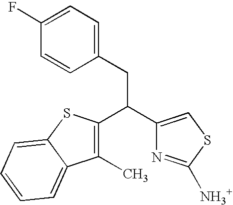 | C | 369.08 |
| 196 | 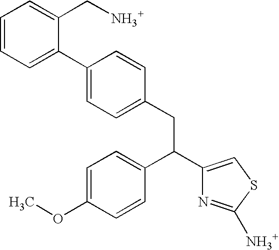 | C | 416.56 |
| 197 | 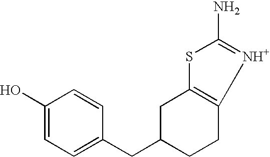 | A | 261.1 |
| 198 | 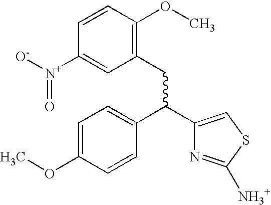 | C | 386.44 |
| 199 | 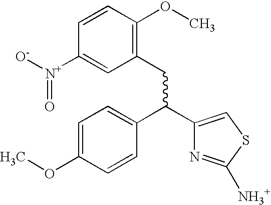 | C | 386.44 |
| 200 | 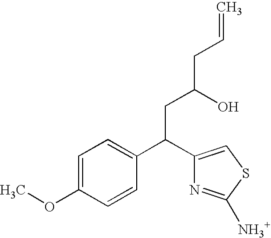 | C | 305.41 |
| 201 | 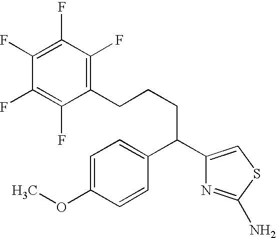 | C | 428.43 |
| 202 | 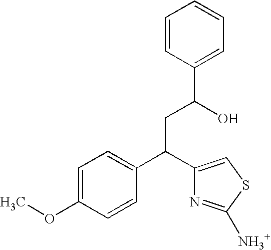 | C | 341.44 |
| 203 | 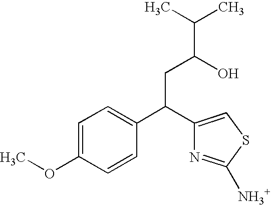 | C | 307.43 |
| 204 | 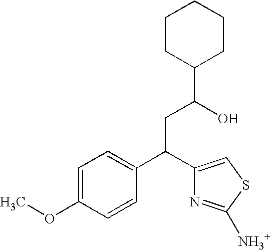 | C | 347.49 |
| 205 | 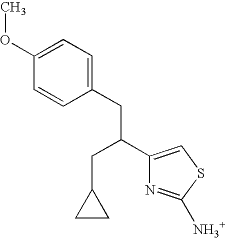 | C | 289.13 |
| 206 | 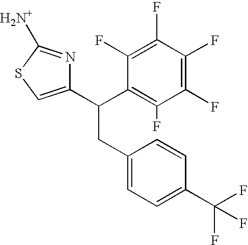 | C | 439.35 |
| 207 | 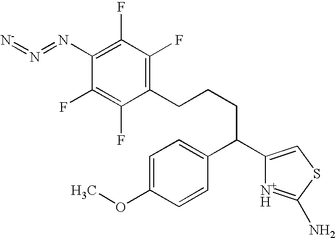 | C | 452.44 |
| 208 | 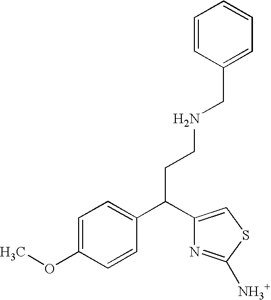 | C | 354.49 |
| 209 | 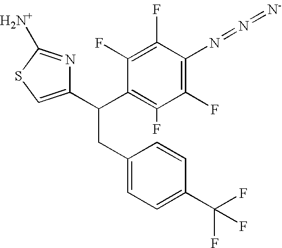 | C | 462.36 |
| 210 | 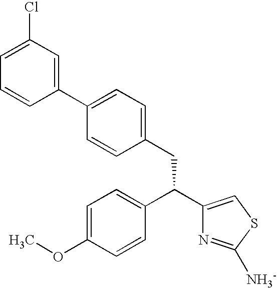 | C | 421.96 |
| 211 | 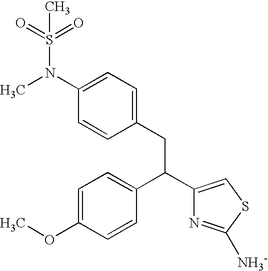 | C | 418.55 |
| 212 | 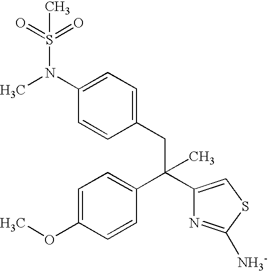 | C | 433.51 |
| 213 | 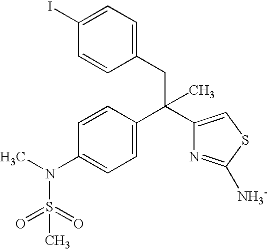 | C | 514.42 |
| 214 | 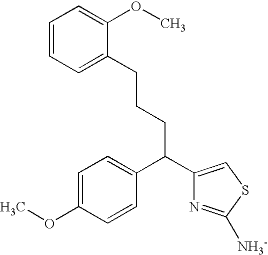 | C | 369.5 |
| 215 | 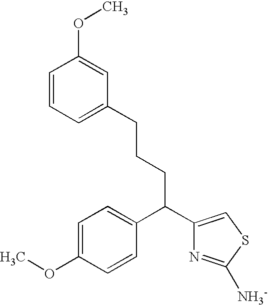 | C | 369.5 |
| 216 | 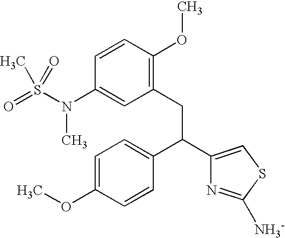 | C | 448.58 |
| 217 | 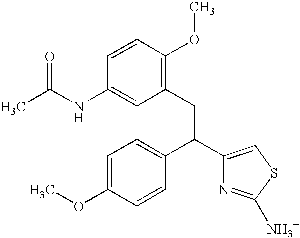 | C | 395.8 |
| 218 | 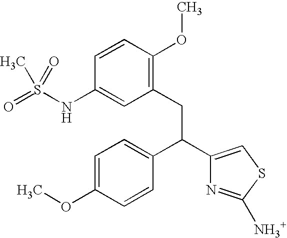 | C | 434.55 |
| 219 | 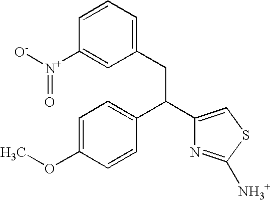 | C | 356.41 |
| 220 | 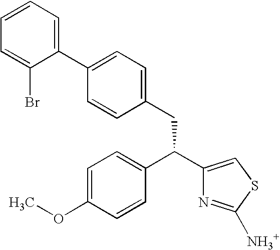 | C | 466.41 |
| 221 | 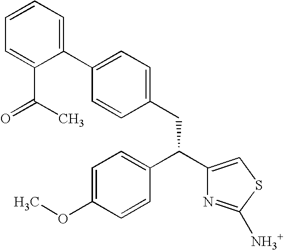 | C | 466.41 |
| 222 | 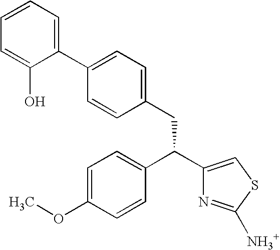 | C | 403.51 |
| 223 | 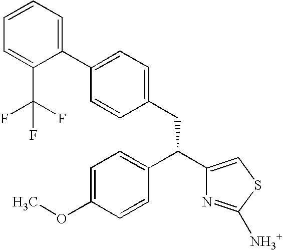 | C | 455.51 |
| 224 | 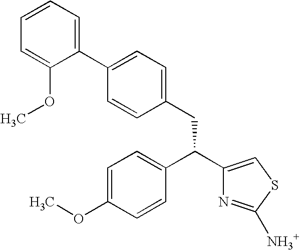 | C | 416.55 |
| 225 | 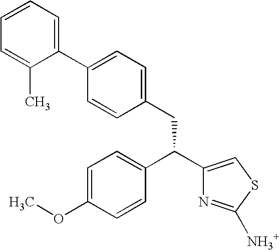 | C | 401.54 |
| 226 | 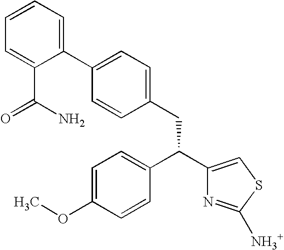 | C | 430.54 |
| 227 | 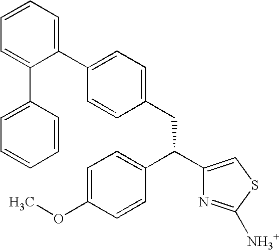 | C | 463.61 |
| 228 | 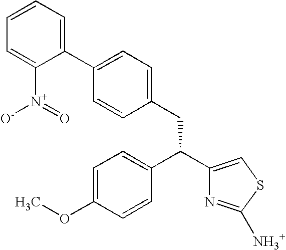 | C | 432.51 |
| 229 | 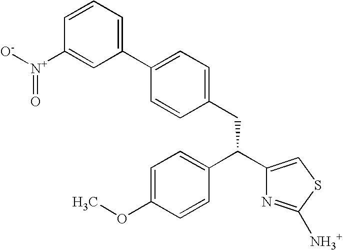 | C | 432.51 |
| 230 | 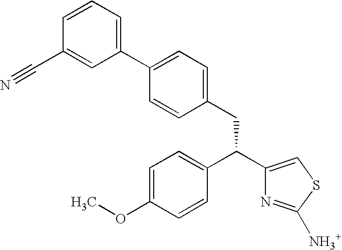 | C | 412.53 |
| 231 | 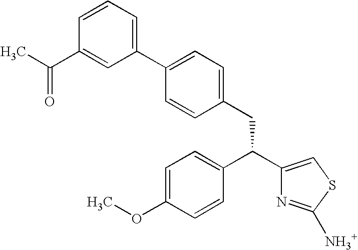 | C | 429.55 |
| 232 | 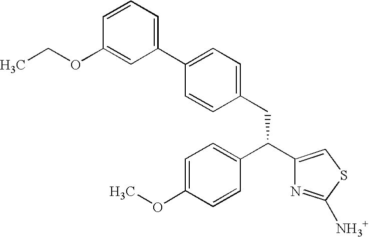 | C | 431.57 |
| 233 | 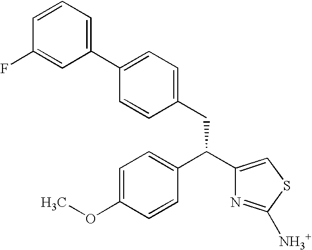 | C | 405.51 |
| 234 | 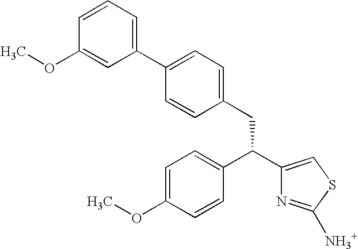 | C | 417.55 |
| 235 | 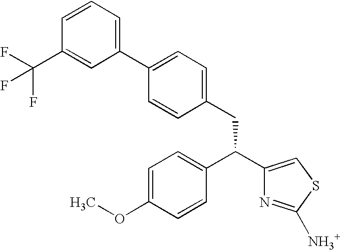 | C | 455.51 |
| 236 | 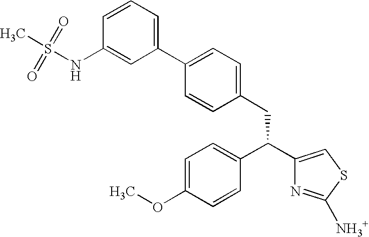 | C | 480.62 |
| 237 | 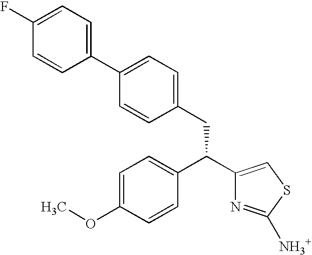 | C | 405.51 |
| 238 | 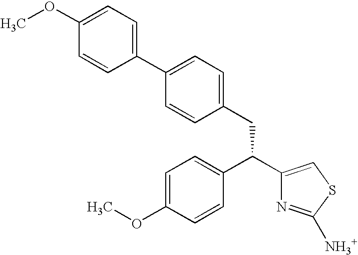 | C | 417.54 |
| 239 | 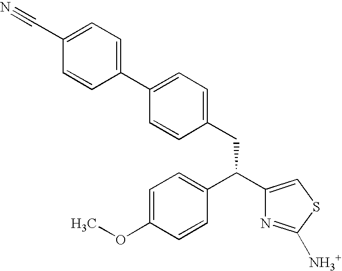 | C | 412.53 |
| 240 | 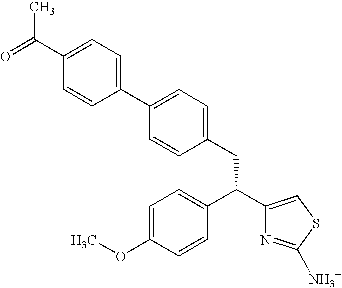 | C | 429.55 |
| 241 | 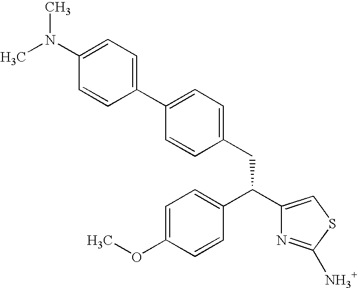 | C | 430.58 |
| 242 | 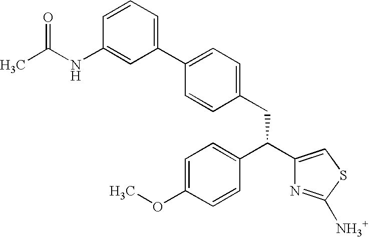 | C | 444.57 |
| 243 | 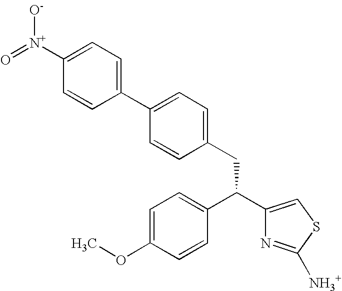 | C | 432.51 |
| 244 | 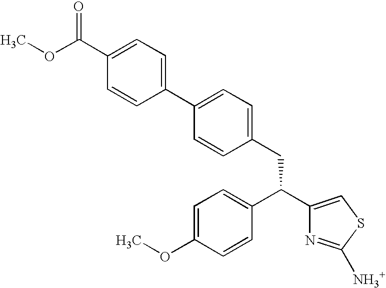 | C | 445.55 |
| 245 | 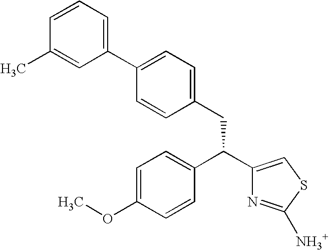 | C | 401.55 |
| 246 | 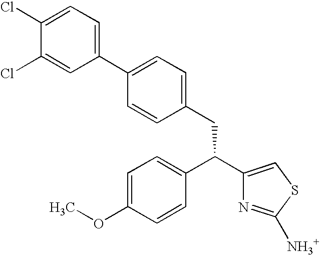 | C | 456.41 |
| 247 | 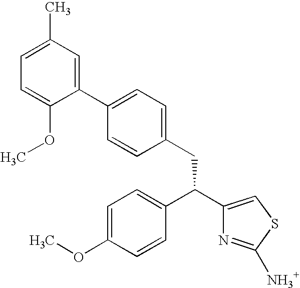 | C | 430.57 |
| 248 | 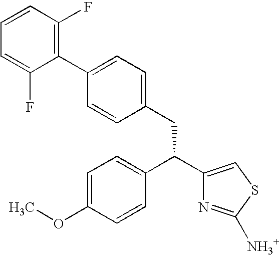 | C | 423.5 |
| 249 | 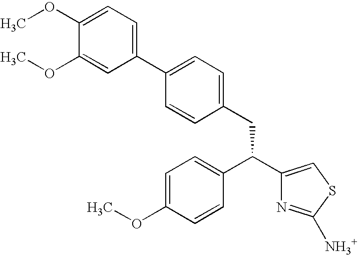 | C | 447.57 |
| 250 | 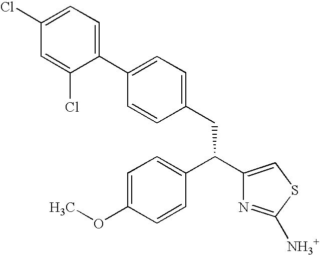 | C | 456.42 |
| 251 | 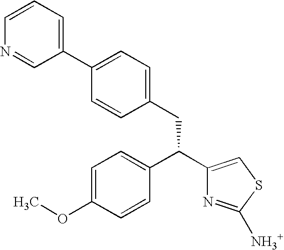 | C | 388.5 |
| 252 | 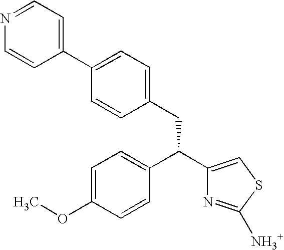 | C | 388.51 |
| 253 | 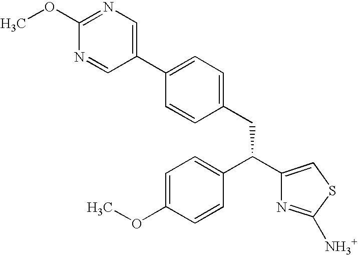 | C | 419.53 |
| 254 | 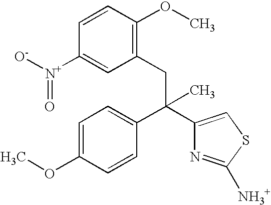 | C | 401.44 |
| 255 | 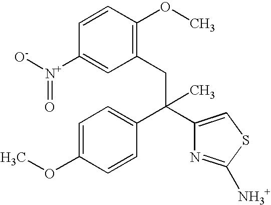 | C | 401.44 |
| 256 | 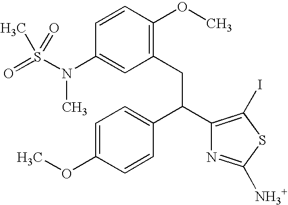 | C | 573.9 |
| 257 | 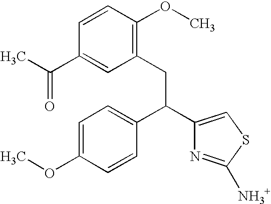 | C | 383.1 |
| 258 | 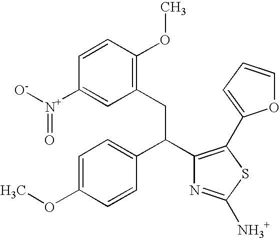 | C | 451.97 |
| 259 | 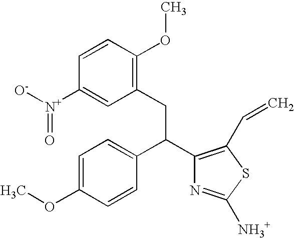 | C | 474 |
| 260 | 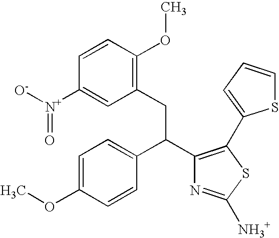 | C | 468 |
| 261 | 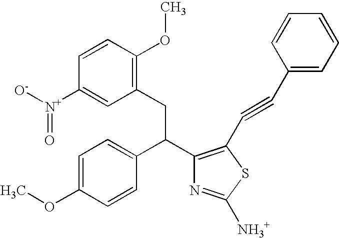 | C | 486 |
| 262 | 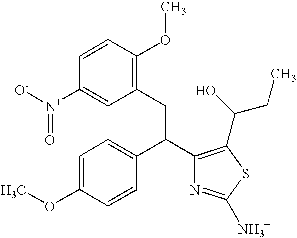 | C | 444 |
| 263 | 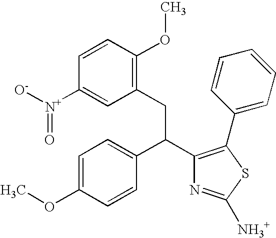 | C | 462 |
| 264 | 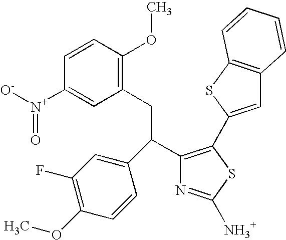 | C | 536.01 |
| 265 | 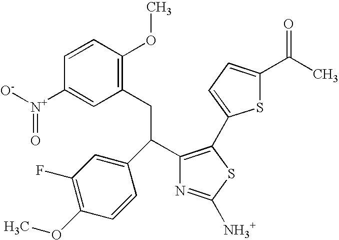 | C | 528.01 |
| 266 | 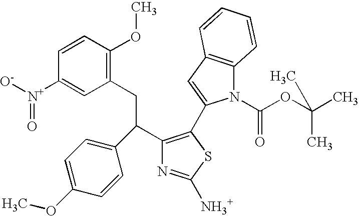 | C | 602.13 |
| 267 | 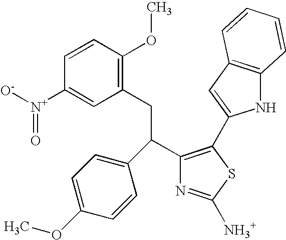 | C | 501.1 |
| 268 | 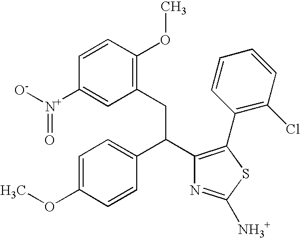 | C | 496.01 |
| 269 | 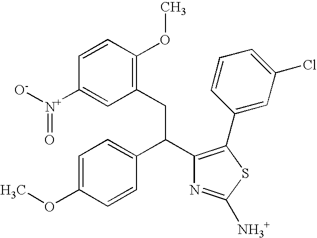 | C | 496.01 |
| 270 | 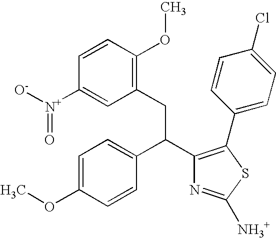 | C | 496.01 |

While some the compounds depicted in the table above are represented in their acid form, the invention is intended to encompass both the salt and free base forms of the compounds described above.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1. A compound of formula (I): 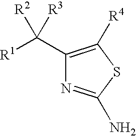
wherein:
R¹ is selected from the group consisting of:
(1) C₁₋₆alkyl,
(2) C₂₋₆ alkenyl,
(3) —C₀₋₆alkylC₃₋₆ cycloalkyl,
(4) 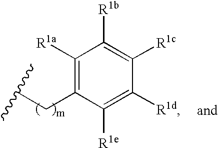
(5) heteroaryl selected from the group consisting of furyl, pyranyl, benzofliranyl, isobenzofuranyl, chromenyl, thienyl, benzothiophenyl, pyrrolyl, pyrazolyl, imidazolyl, pyridyl, pyrazinyl, pyrimidinyl, pyridazinyl, indolyl, indazolyl, benzimidazolyl, quinolyl and isoquinolyl,
wherein
(a) said alkyl, alkenyl or cycloalkyl is unsubstituted or substituted with one or more halogen, —C₁₋₆alkyl, —C₁₋₆alkoxy, hydroxy or cyano, and
(b) said heteroaryl is unsubstituted or substituted with one or more halogen, —C₁₋₆alkyl, —C₁₋₆alkoxy, phenyl, hydroxy or cyano,
and wherein R^{1a}, R^{1b}, R^{1c}, R^{1d} and R^{1e} are selected from the group consisting of:
(a) hydrogen,
(b) halogen,
(c) cyano,
(d) hydroxyl,
(e) —C₁₋₆ alkoxy,
(f) —C(═O)OR^{7a},
(g) —C₀₋₆alkyl-C(═O)—R^{7a},
(h) —N—R^{7a}—S(O)ₚ—R^{7b},
or R^{1b} and R^{1c} are linked together to form —O—CH₂—O— or —CH═CH—CH═CH—;
wherein said aryl is unsubstituted or substituted with one or more halogen, —C₁₋₆alkyl, —C₁₋₆alkoxy, hydroxyl or cyano;
R² is selected from the group consisting of:
(1) hydrogen,
(2) halogen,
(3) —C₀₋₆alkyl-Q¹-C₁₋₆alkyl, wherein Q¹ is O or S,
(4) —C₁₋₆alkyl, and
(5) hydroxyl;
R³ is selected from the group consisting of:
(1) hydrogen,
(2) —C₁₋₆alkyl,
(3) —C₀₋₆alkyl-C₃₋₆cycloalkyl,
(4) —C₀₋₆alkyl-Q²-C₁₋₆alkyl, wherein Q² is O, S or —C(═O)—O—, and
(5) 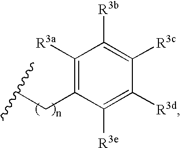
(6) —CH₂-heteroaryl, wherein said heteroaryl is selected from the group consisting of furyl, pyranyl, benzofuranyl, isobenzofuranyl, chromenyl, thienyl, benzothiophenyl, pyrrolyl, pyrazolyl, imidazolyl, pyridyl, pyrazinyl, pyrimidinyl, pyridazinyl, indolyl, indazolyl, benzimidazolyl, quinolyl and isoquinolyl,
wherein said alkyl or cycloalkyl is unsubstituted or substituted with one or more
(a) halogen,
(b) —C₁₋₆alkyl,
(c) —C₂₋₆alkenyl,
(d) —C₁₋₆alkoxy,
(e) —C₆₋₁₀ aryl,
(f) hydroxyl, or
(g) cyano,
and said heteroaryl is unsubstituted or substituted with one or more
(a) —C₁₋₆alkyl,
(b) —NR^{3f}R^{3g}, wherein R^{3f} and R^{3g} are selected from the group consisting of:
(i) hydrogen,
(ii) —C₁₋₆ alkyl,
(iii) —C₁₋₆alkyl-C₆₋₁₀ aryl, wherein said aryl can be substituted or unsubstited with halogen, cyano, C₁₋₆ alkyl or C₁₋₆ alkoxy, or
(iv) —C₁₋₆alkyl-NR^{7a}R^{7b},
or N, R^{3f} and R^{3g} together form a 5 or 6 membered heterocyclic group, optionally containing an N, S or O atom in addition to the N atom attached to R^{3f} and R^{3g};
and R^{3a}, R^{3b}, R^{3c}, R^{3d} and R^{3e} are selected from the group consisting of:
(i) hydrogen,
(ii) halogen,
(iii) cyano,
(iv) hydroxyl,
(v) —C₁₋₆ alkyl,
(vi) —O—R^{7a},
(vii) —(C═O)—O—R⁸,
(viii) —NR^{7a—S(O)}ₚOR^{7b},
(ix) —NR^{7a}—S(O)ₚR^{7b},
(x) —C₀₋₆alkyl —S(O)ₘR^{7a},
(xi) —C(═O)NR^{7a}R^{7b},
(xii) —C(═O)—R⁸
(xiii) —NH—C(═O)—R^{7a},
(xiv) —C₀₋₆alkyl-NR^{7a}R^{7b},
(xv) —N₃,
(xvi) —NO₂,
(xvii) C₆₋₁₀ aryl, wherein said aryl can be unsubstituted or substituted with one or more
(A) halogen,
(B) cyano,
(C) —C₁₋₆ alkyl,
(D) —C₁₋₆ alkoxy,
(E) —C(═O)—O—R^{7a},
(F) —C(═O)—R^{7a},
(G) —NR^{7a}R^{7b},
(H) —NR^{7a}—S(O)ₚ—R^{7b},
(I) —NR^{7a}—C(═O)—R^{7b},
(J) —NO₂
(xviii) heteroaryl selected from the group consisting of furyl,pyranyl, benzofuiranyl, isobenzofuiranyl, chromenyl, thienyl, benzothiophenyl, pyrrolyl, pyrazolyl, imidazolyl, pyridyl, pyrazinyl, pyrimidinyl, pyridazinyl, indolyl, indazolyl, benzimidazolyl, quinolyl and isoquinolyl,
wherein said heteroaryl is unsubstituted or substituted with one or more
(A) —C₁₋₆ alkyl, or
(B) —C₁₋₆ alkoxy;
or R^{3c} and R^{3d} are linked together to form phenyl or the group —O—CH₂—O— or —CH═CH—CH═CH—;
or R² and R³ are linked to form a carbocyclic ring (A): 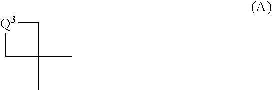
wherein Q³ is selected from the group consisting of:
(1) —CR^{7a}R^{7b}—,
(2) —CR^{7a}R^{7b}CR^{7c}R^{7d}—,
(3) —CR^{7a}═CR^{7b}—,
(4) —CR^{7a}R^{7b}CR^{7c}R^{7d}CR^{7e}R^{7f}—,
(5) —CR^{7a}═CR^{7b}CR^{7c}R^{7d}—, and
(6) —CR^{7a}R^{7b}CR^{7d}═CR^{7e}—;
R⁴ is selected from the group consisting of:
(1) hydrogen,
(2) halogen,
(3) —C₁₋₆alkyl,
(4) —C₂₋₆alkenyl,
(5) —C₂₋₆alkynyl,
(6) phenyl,
(7) benzyl, and
(8) heteroaryl selected from the group consisting of furyl, pyranyl, benzofuranyl, isobenzofuranyl, chromenyl, thienyl, benzothiophenyl, pyrrolyl, pyrazolyl, imidazolyl, pyridyl, pyrazinyl, pyrimidinyl, pyridazinyl, indolyl, indazolyl, benzimidazolyl, quinolyl and isoquinolyl,
wherein said alkyl, alkenyl, alkynyl and phenyl is unsubstituted or substituted with one or more
(a) halogen,
(b) cyano,
(c) hydroxyl,
(d) phenyl,
(e) —C₁₋₆ alkyl,
(f) —C₁₋₆ alkoxy,
(g) —C(═O)—O—R^{7a},
(h) —C(═O)—R^{7a},
(i) —NR^{7a}R^{7b},
(j) —NR^{7a}—S(O)ₚ—R^{7b},
(k) —NR^{7a}—C(═O)—R^{7b},
(l) —NO₂;
and said heteroaryl is unsubstituted or substituted with one or more:
(a) —C₁₋₆alkyl,
(b) —C(═O)—O—R^{7a}
(c) —C(═O)—R^{7a}
(d) —NR^{3f}R^{3g}, wherein R^{3f} and R^{3g} selected from the group consisting of
(i) hydrogen,
(ii) —C₁₋₆ alkyl,
(iii) —C₁₋₆alkyl-C₆₋₁₀ aryl, wherein said aryl can be substituted or unsubstited with halogen, cyano, C₁₋₆ alkyl or C₁₋₆ alkoxy, or
(iv) —C₁₋₆alkyl-NR^{7a}R^{7b};
or R³ and R⁴ may be joined together to form a 6-membered carbocyclic ring (B): 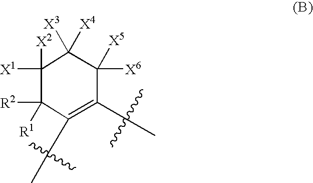
provided that when R³ and R⁴ are joined together to form (B) then R¹ and R² are selected from the group consisting of hydrogen or C₁₋₆ alkyl, and X¹, X², X³, X⁴, X⁵ and X⁶ are selected from the consisting of hydrogen, C₁₋₆ alkyl, C₃₋₆ cycloalkyl, cyano, alkylaryl or phenyl,
or R³ and R⁴ may be joined together to form a 7-membered carbocyclic ring (C): 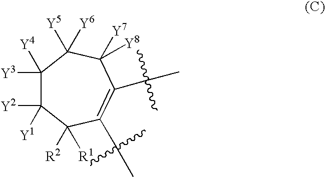
provided that when R³ and R⁴ are joined together to form (C) then R¹ and R² are selected from the group consisting of hydrogen, C₁₋₆ alkyl or phenyl, or R¹ and R² can be linked together by the group —CH₂CH₂CH₂CH₂—; and Y¹, Y², Y³, Y⁴, Y⁵, Y⁶, Y⁷ and Y⁸ are selected from the group consisting of hydrogen, C₁₋₆ alkyl, C₃₋₆ cycloalkyl, cyano, alkylaryl or phenyl,
or R¹ and Y⁵, or R¹ and Y⁷, are linked together by —CH₂—,
or R¹ and Y¹, or Y¹ and Y³, are linked together to form a phenyl or cyclopentyl ring;
R^{7a}, R^{7b}, R^{7c}, R^{7d}, R^{7e} and R^{7f} are selected from the group consisting of:
(1) hydrogen,
(2) C₁₋₆ alkyl, and
(3) C₆₋₁₀ aryl;
wherein said alkyl or aryl is unsubstituted or substituted with one or more halogen, —C₁₋₆alkyl, —C₁₋₆alkoxy, hydroxyl or cyano;
R⁸ is selected from the group consisting of:
(1) hydrogen,
(2) C₁₋₆ alkyl, and
(3) C₆₋₁₀ aryl, wherein said aryl is unsubstituted or substituted with one or more halogen, —C₁₋₆alkyl, —C₁₋₆alkoxy, hydroxy or cyano;
n is 0, 1, 2 or 3
m is 0 or 1;
p is 1 or 2;
and pharmaceutically acceptable salts thereof, and individual enantiomers and diastereomers thereof.

2. The compound of claim 1 wherein R³ is selected from the group consisting of:
(1) —C₁₋₆alkyl,
(2) —C₀₋₆alkyl-C₃₋₆cycloalkyl,
(3) 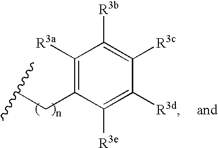
(4) —CH₂-heteroaryl, wherein said heteroaryl is selected from the group consisting of furyl, pyranyl, benzofuiranyl, isobenzofuranyl, chromenyl, thienyl, benzothiophenyl, pyrrolyl, pyrazolyl, imidazolyl, pyridyl,pyrazinyl, pyrimidinyl, pyridazinyl, indolyl, indazolyl, benzimidazolyl, quinolyl and isoquinolyl.

3. The compound of claim 2 wherein R³ is 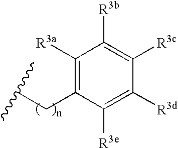
and n is 1.

4. The compound of claim 2 wherein R¹ is 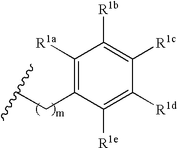
and m is 0.

5. The compound of claim 4 wherein R^{1a}, R^{1b}, R^{1d} and R^{1e} are hydrogen, and R^{1c} is selected from the group consisting of halogen, C₁₋₆ alkyl and C₁₋₆ alkoxy.

6. The compound of claim 2 wherein R² is hydrogen.

7. The compound of claim 2 wherein R⁴ is hydrogen.

8. The compound of claim 1 which is a compound of formula (III)

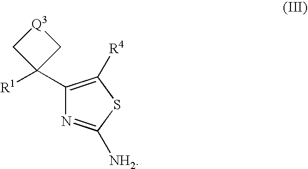

9. The compound of claim 8 wherein R¹ is 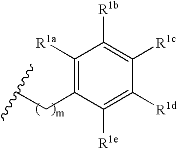
and m is 0.

10. The compound of claim 9 wherein Q³ is selected from the group consisting of
(1) —CR^{7a}R^{7b}—,
(2) —CR^{7a}R^{7b}CR^{7c}R^{7d}—, and
(3) —CR^{7a}R^{7b}CR^{7c}R^{7d}CR^{7e}R^{7f}—.

11. The compound of claim 10 wherein R^{1d} is selected from the group consisting of halogen, C₁₋₆ alkyl, C₁₋₆ alkoxy and cyano, and R^{1a}, R^{1b}, R^{1c} and R^{1e} are hydrogen.

12. The compound of claim 9 wherein R^{1b} and R^{1d} are selected from the group consisting of halogen, C₁₋₆ alkyl, C₁₋₆ alkoxy and cyano, and R^{1a}, R^{1c} and R^{1e} are hydrogen.

13. The compound of claim 8 wherein Q³ is selected from the group consisting of —CH₂CH₂— and —CH₂CH₂CH₂—.

14. The compound of claim I which is a compound of formula (IV)

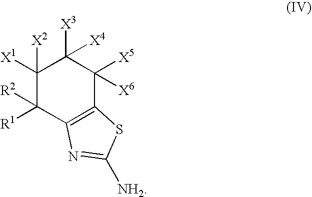

15. The compound of claim 14 wherein R¹ and R² are hydrogen.

16. The compound of claim 1 which is a compound of formula (V)

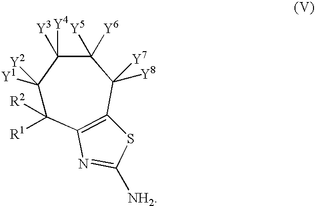

17. The compound of claim 1 which is selected from the group consisting of 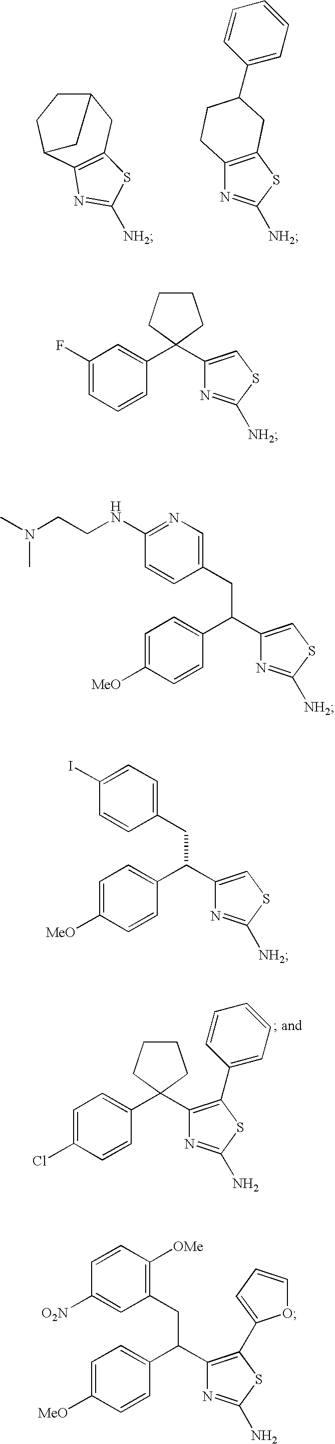
and pharmaceutically acceptable salts thereof.

18. The compound of claim 1 which is selected from the group consisting of

| Example | Structure |
|---|---|
| 8 | 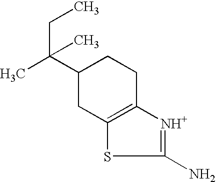 |
| 9 | 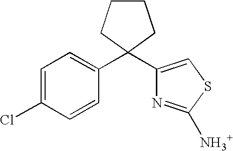 |
| 10 | 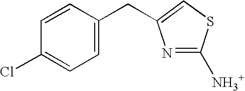 |
| 11 | 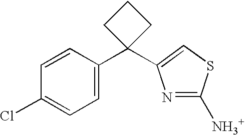 |
| 12 | 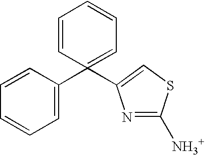 |
| 13 | 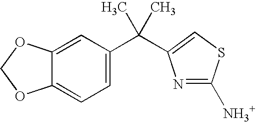 |
| 14 | 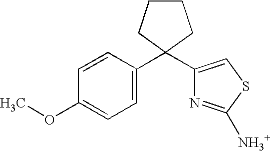 |
| 15 | 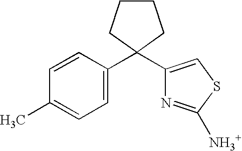 |
| 16 | 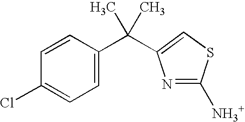 |
| 17 | 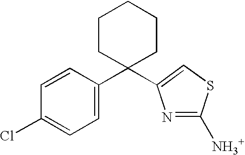 |
| 18 | 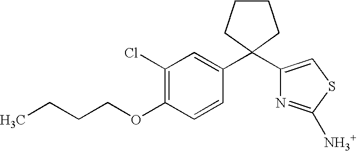 |
| 19 | 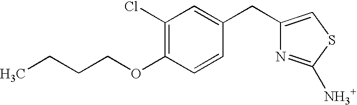 |
| 20 | 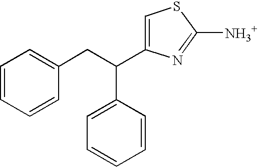 |
| 21 | 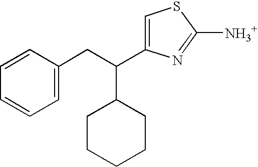 |
| 22 | 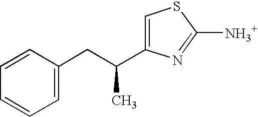 |
| 23 | 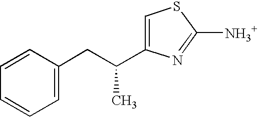 |
| 24 | 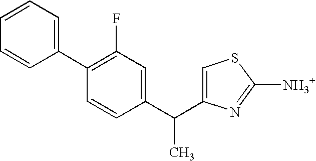 |
| 25 | 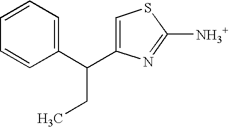 |
| 26 | 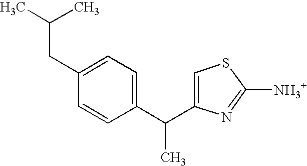 |
| 27 | 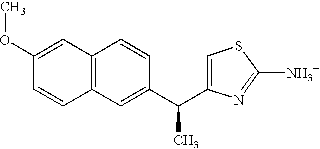 |
| 28 | 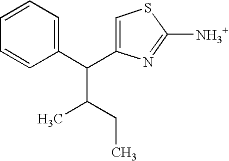 |
| 29 | 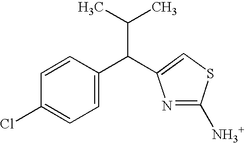 |
| 30 | 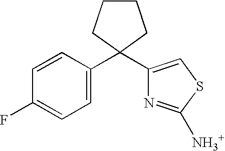 |
| 31 | 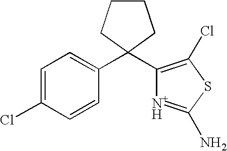 |
| 32 | 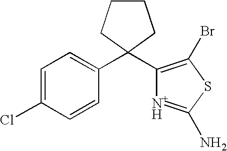 |
| 33 | 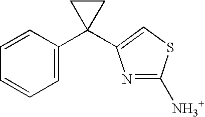 |
| 34 | 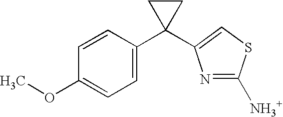 |
| 35 | 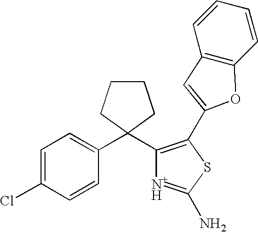 |
| 36 | 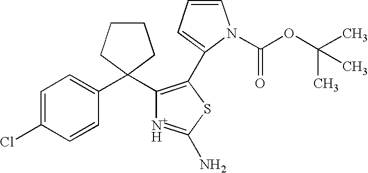 |
| 37 | 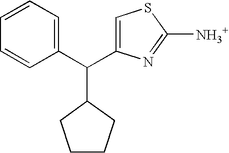 |
| 38 | 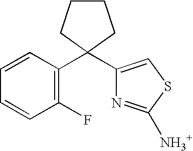 |
| 39 | 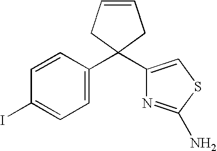 |
| 40 | 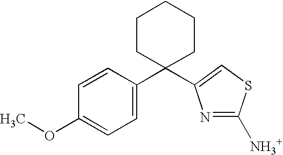 |
| 41 | 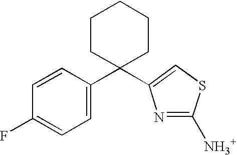 |
| 42 | 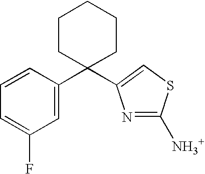 |
| 43 | 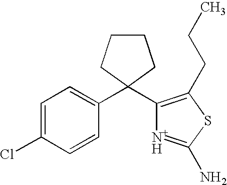 |
| 44 | 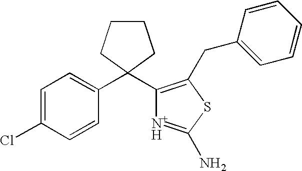 |
| 45 | 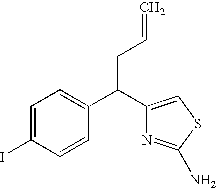 |
| 46 | 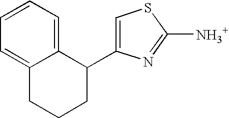 |
| 47 | 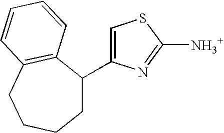 |
| 48 | 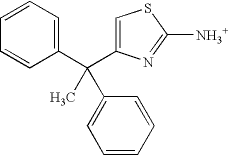 |
| 49 | 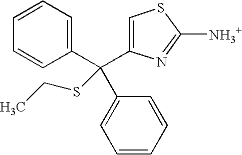 |
| 50 | 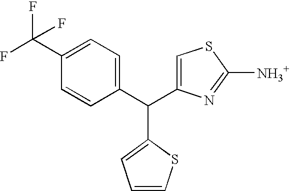 |
| 51 | 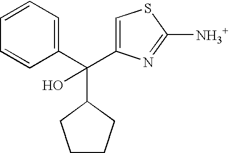 |
| 52 | 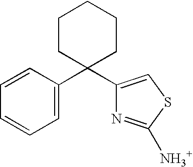 |
| 53 | 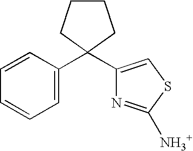 |
| 54 | 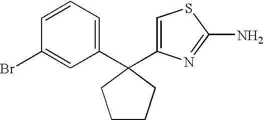 |
| 55 | 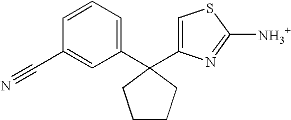 |
| 56 | 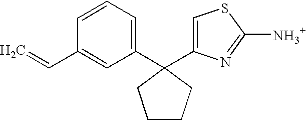 |
| 57 | 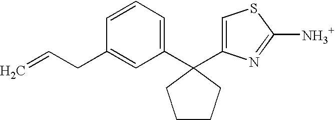 |
| 58 | 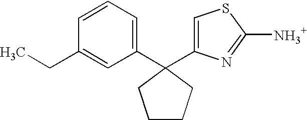 |
| 59 | 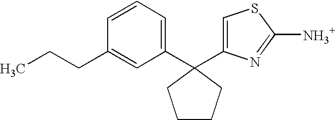 |
| 60 | 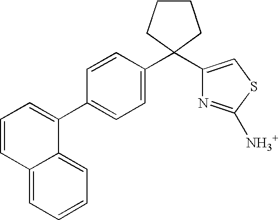 |
| 61 | 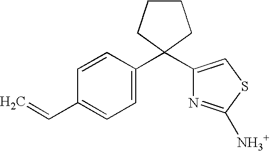 |
| 62 | 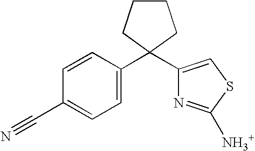 |
| 63 | 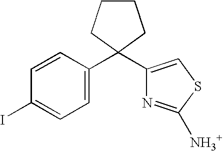 |
| 64 | 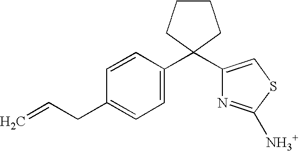 |
| 65 | 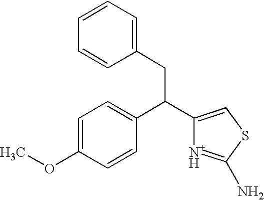 |
| 66 | 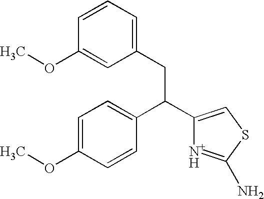 |
| 67 | 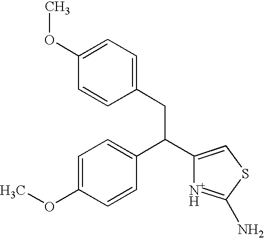 |
| 68 | 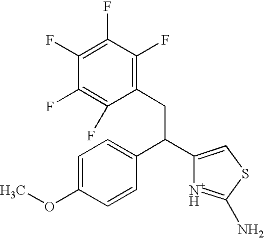 |
| 69 | 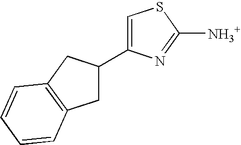 |
| 70 | 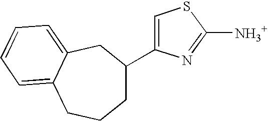 |
| 71 | 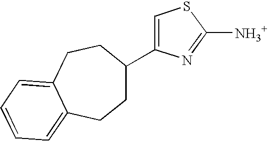 |
| 72 | 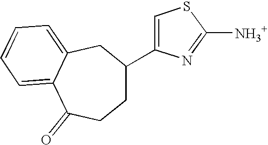 |
| 73 | 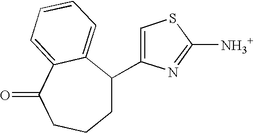 |
| 74 | 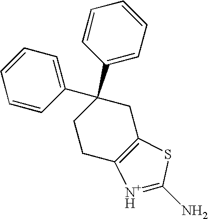 |
| 75 | 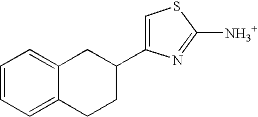 |
| 76 | 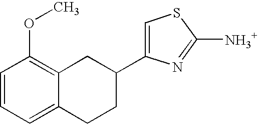 |
| 77 | 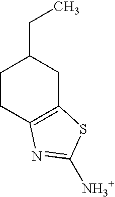 |
| 78 | 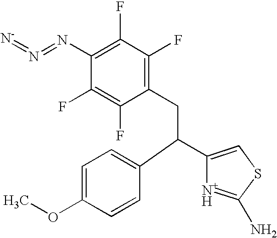 |
| 79 | 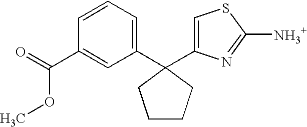 |
| 80 | 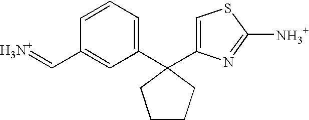 |
| 81 | 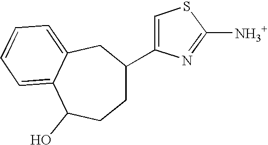 |
| 82 | 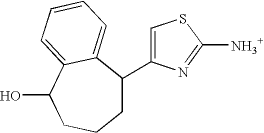 |
| 83 | 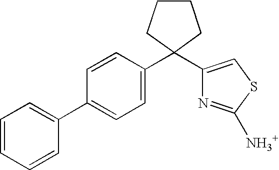 |
| 84 | 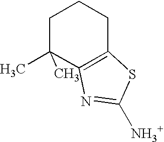 |
| 85 | 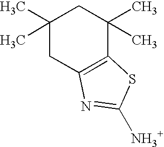 |
| 86 | 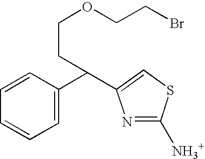 |
| 87 | 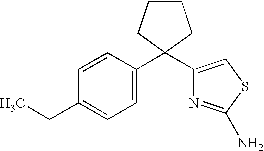 |
| 88 | 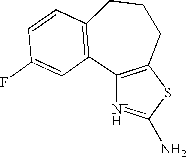 |
| 89 | 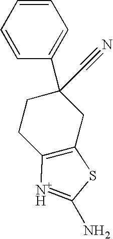 |
| 90 | 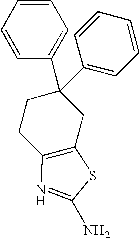 |
| 91 | 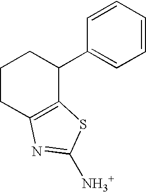 |
| 92 | 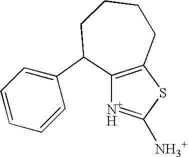 |
| 93 | 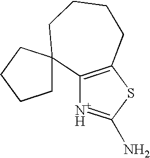 |
| 94 | 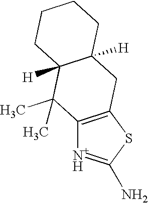 |
| 95 | 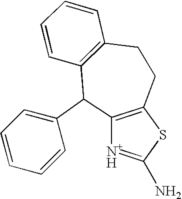 |
| 96 | 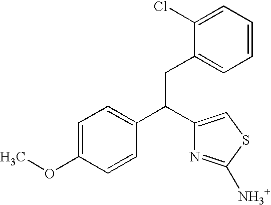 |
| 97 | 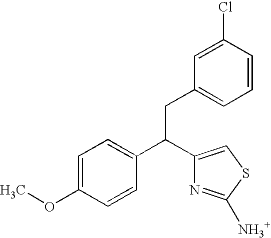 |
| 98 | 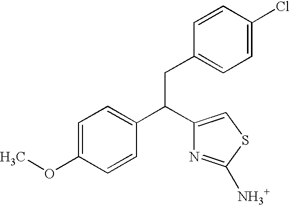 |
| 99 | 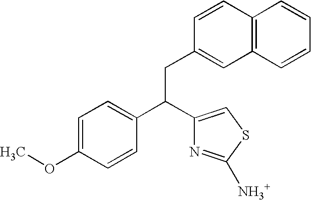 |
| 100 | 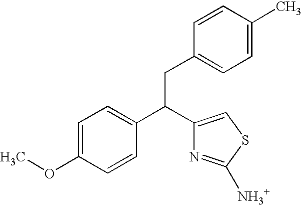 |
| 101 | 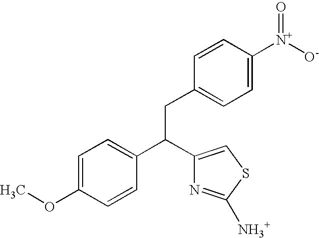 |
| 102 | 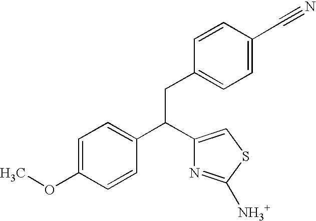 |
| 103 | 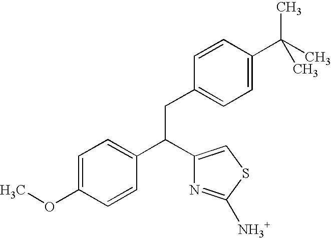 |
| 104 | 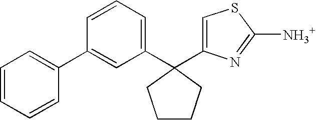 |
| 105 | 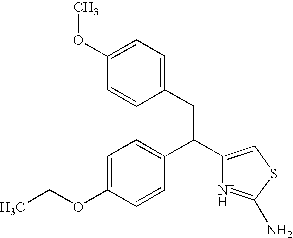 |
| 106 | 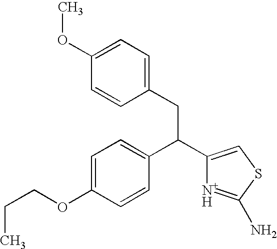 |
| 107 | 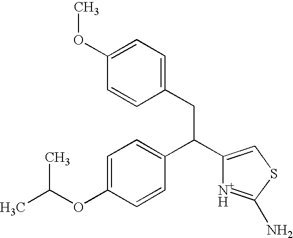 |
| 108 | 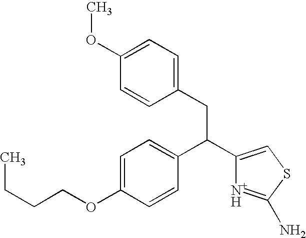 |
| 109 | 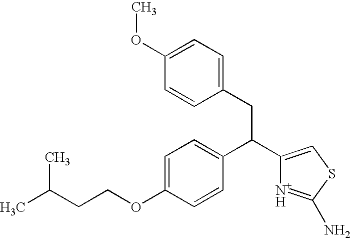 |
| 110 | 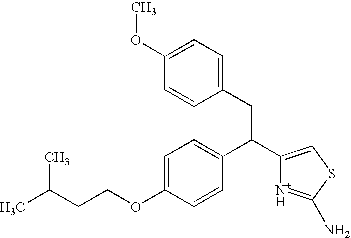 |
| 111 | 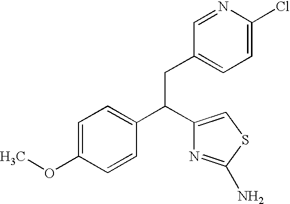 |
| 112 | 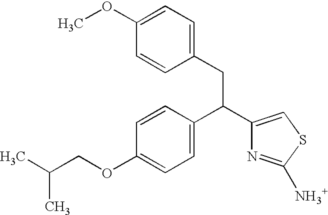 |
| 113 | 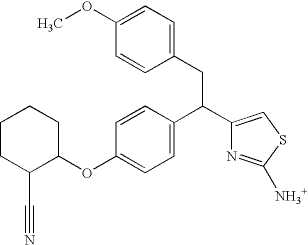 |
| 114 | 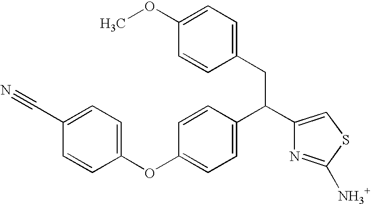 |
| 115 | 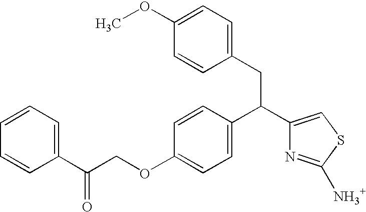 |
| 116 | 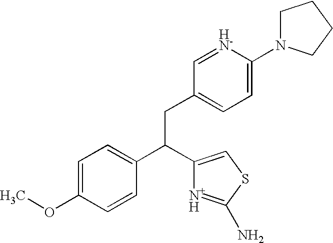 |
| 117 | 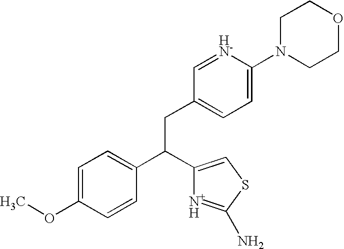 |
| 118 | 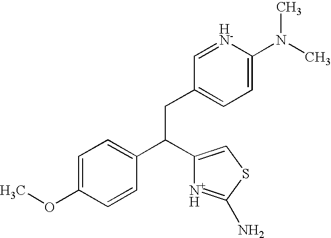 |
| 119 | 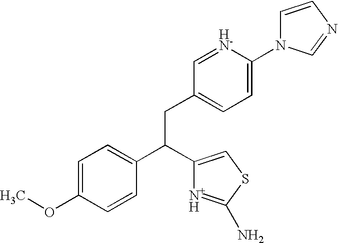 |
| 120 | 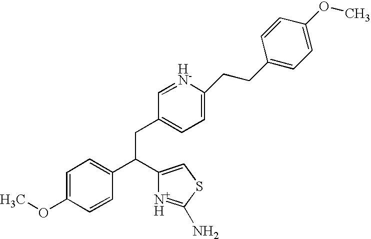 |
| 121 | 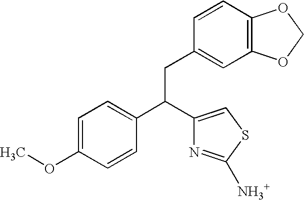 |
| 122 | 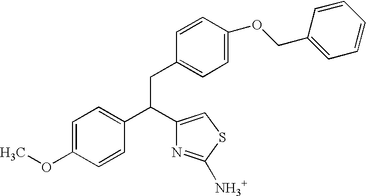 |
| 123 | 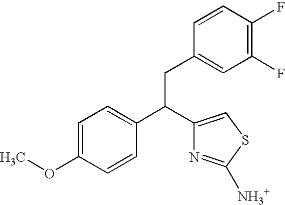 |
| 124 | 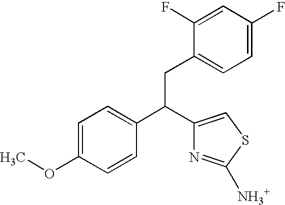 |
| 125 | 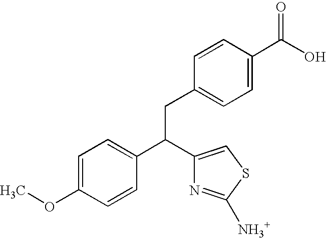 |
| 126 | 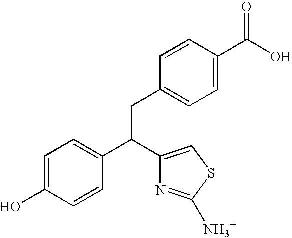 |
| 127 | 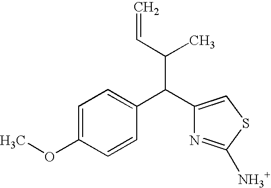 |
| 128 | 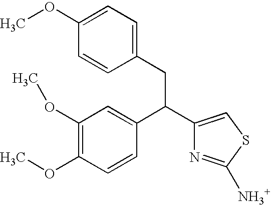 |
| 129 | 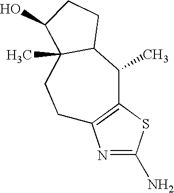 |
| 130 | 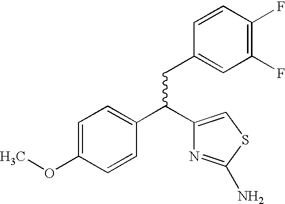 |
| 131 | 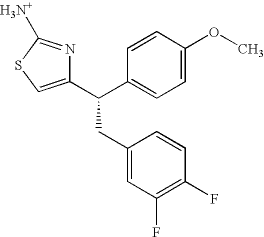 |
| 132 | 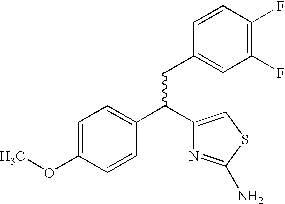 |
| 133 | 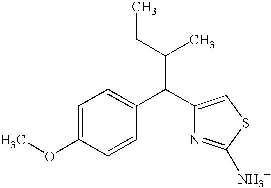 |
| 134 | 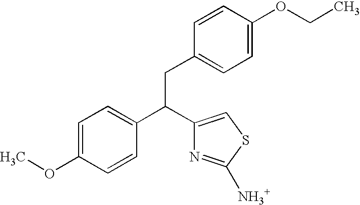 |
| 135 | 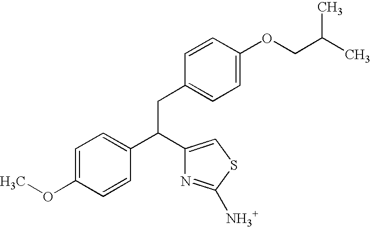 |
| 136 | 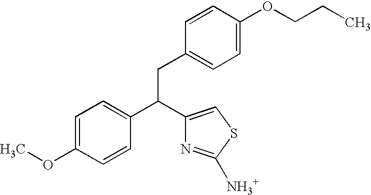 |
| 137 | 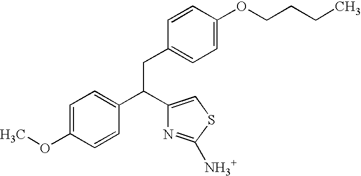 |
| 138 | 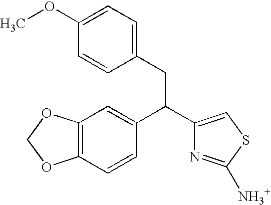 |
| 139 | 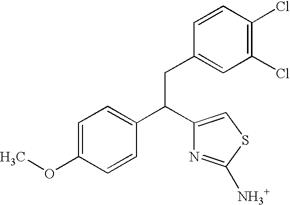 |
| 140 | 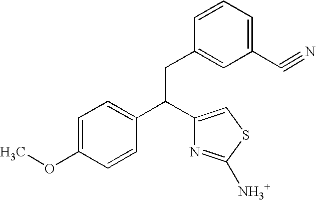 |
| 141 | 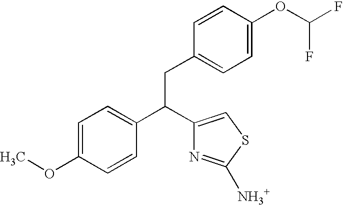 |
| 142 | 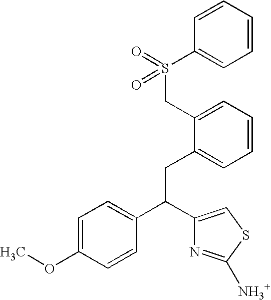 |
| 143 | 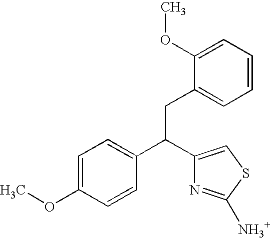 |
| 144 | 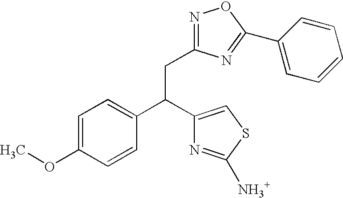 |
| 145 | 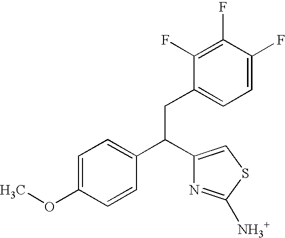 |
| 146 | 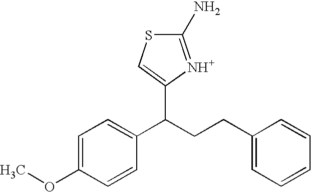 |
| 147 | 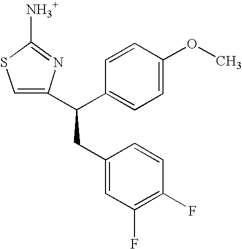 |
| 148 | 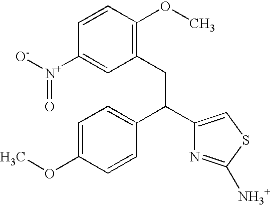 |
| 149 | 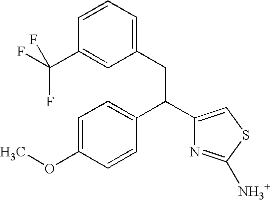 |
| 150 | 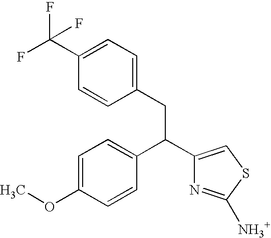 |
| 151 | 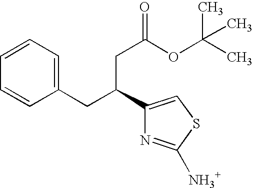 |
| 152 | 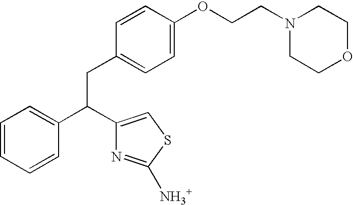 |
| 153 | 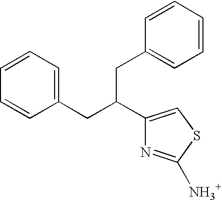 |
| 154 | 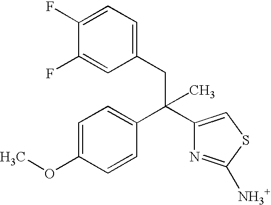 |
| 155 | 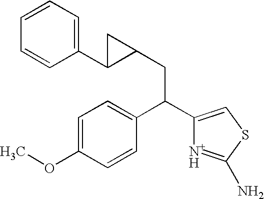 |
| 156 | 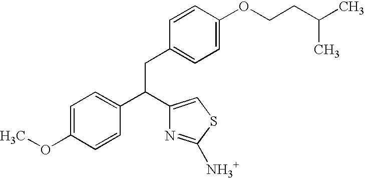 |
| 157 | 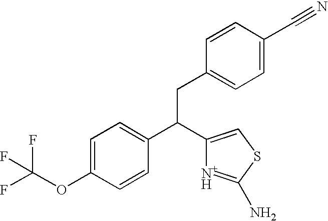 |
| 158 | 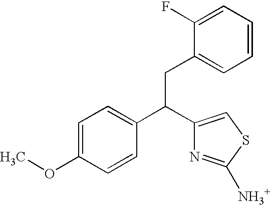 |
| 159 | 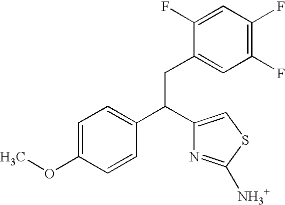 |
| 160 | 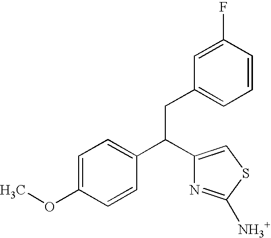 |
| 161 | 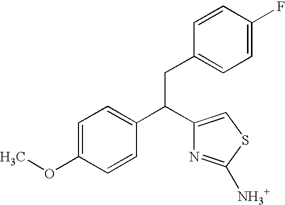 |
| 162 | 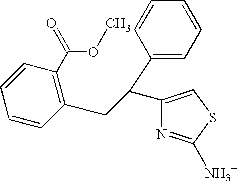 |
| 163 | 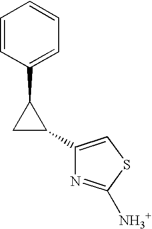 |
| 164 | 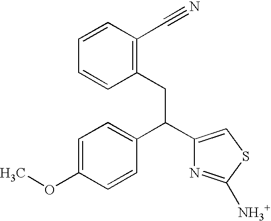 |
| 165 | 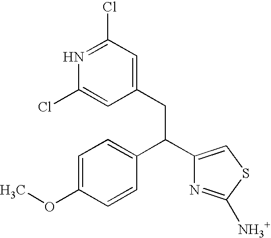 |
| 166 | 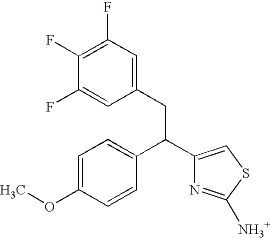 |
| 167 | 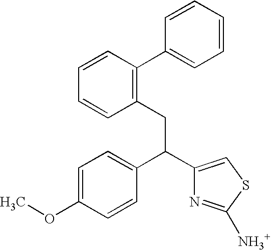 |
| 168 | 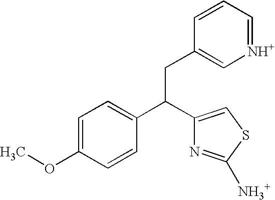 |
| 169 | 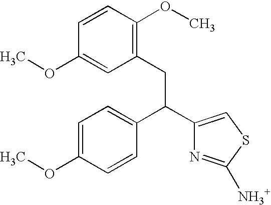 |
| 170 | 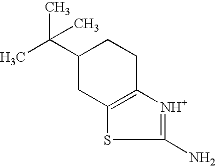 |
| 171 | 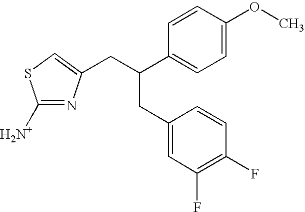 |
| 172 | 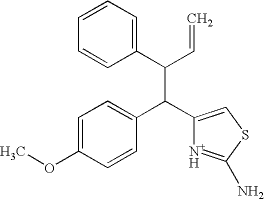 |
| 173 | 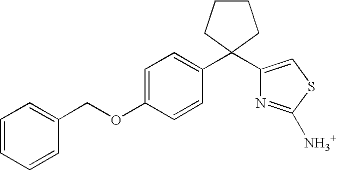 |
| 174 | 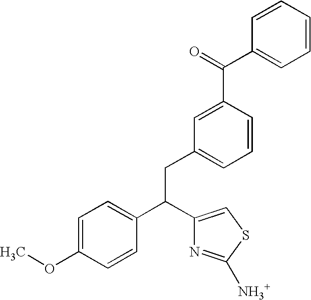 |
| 175 | 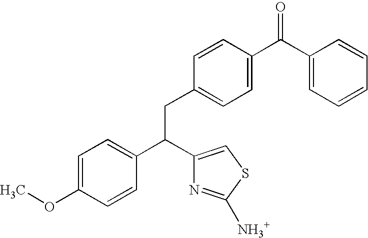 |
| 176 | 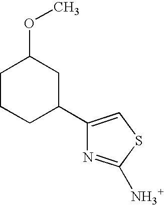 |
| 177 | 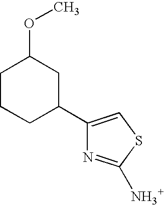 |
| 178 | 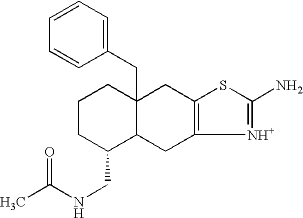 |
| 179 | 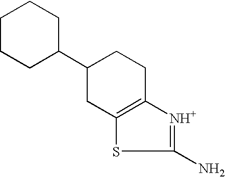 |
| 180 | 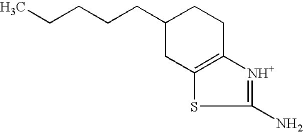 |
| 181 | 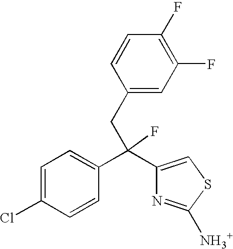 |
| 182 | 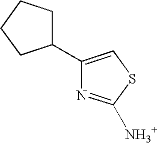 |
| 183 | 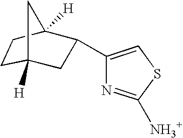 |
| 184 | 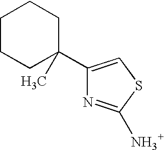 |
| 185 | 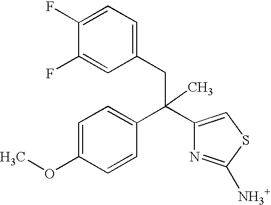 |
| 186 | 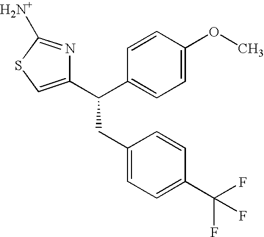 |
| 187 | 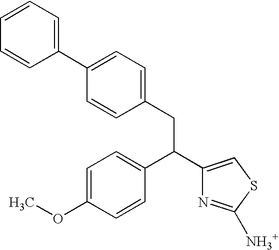 |
| 188 | 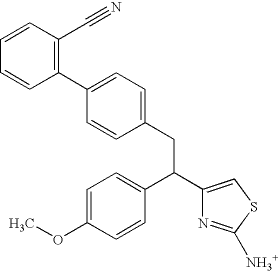 |
| 189 | 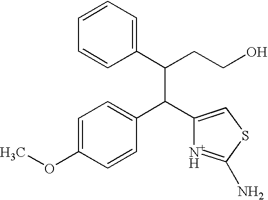 |
| 190 | 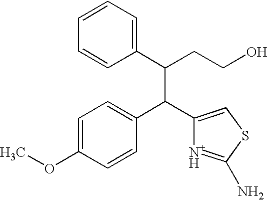 |
| 191 | 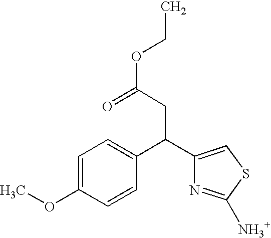 |
| 192 | 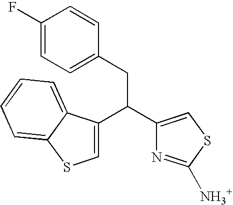 |
| 193 | 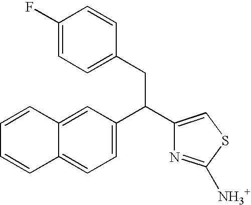 |
| 194 | 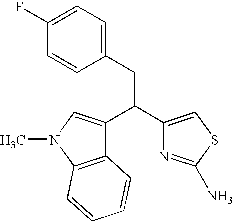 |
| 195 | 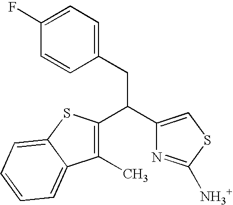 |
| 196 | 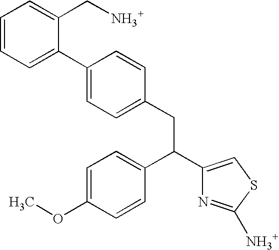 |
| 197 | 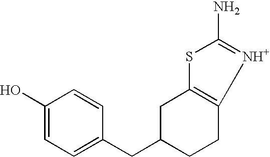 |
| 198 | 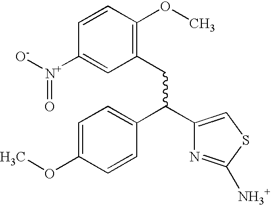 |
| 199 | 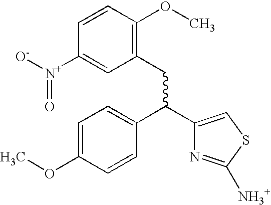 |
| 200 | 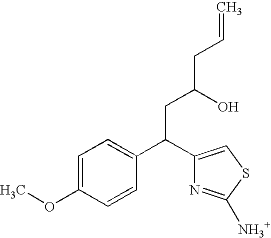 |
| 201 | 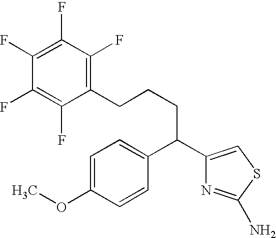 |
| 202 | 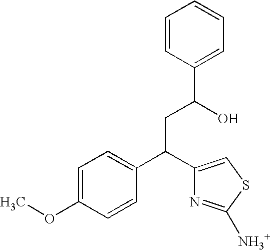 |
| 203 | 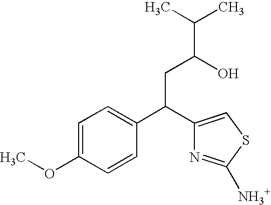 |
| 204 | 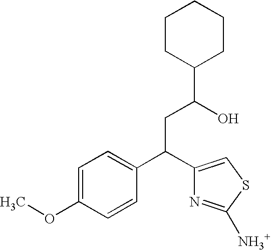 |
| 205 | 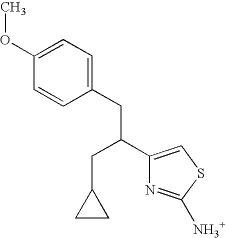 |
| 206 | 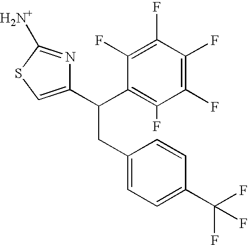 |
| 207 | 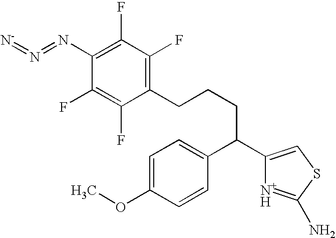 |
| 208 | 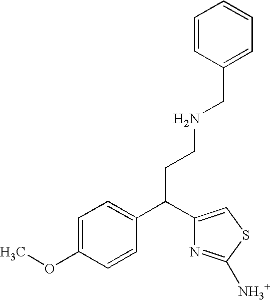 |
| 209 | 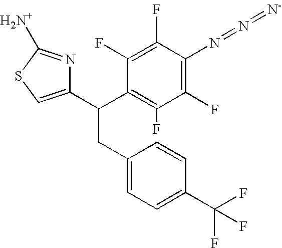 |
| 210 | 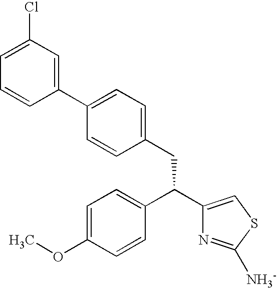 |
| 211 | 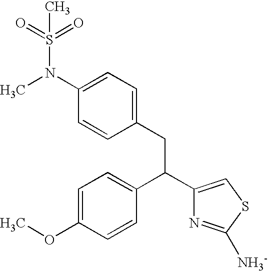 |
| 212 | 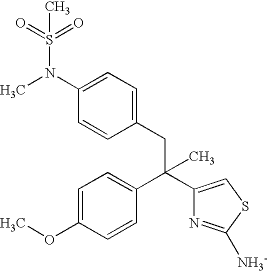 |
| 213 | 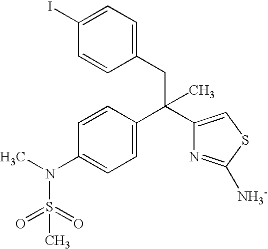 |
| 214 | 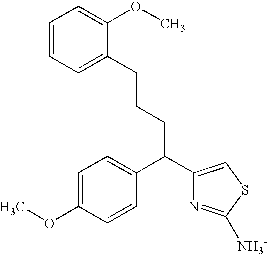 |
| 215 | 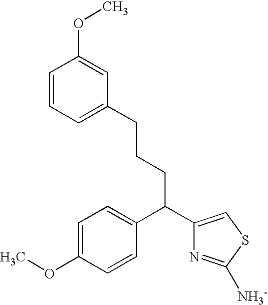 |
| 216 | 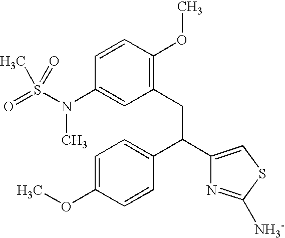 |
| 217 | 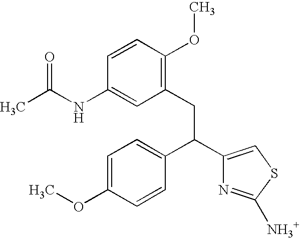 |
| 218 | 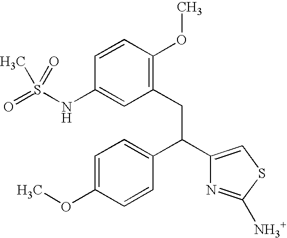 |
| 219 | 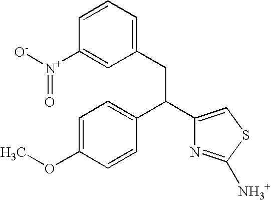 |
| 220 | 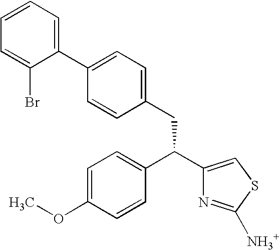 |
| 221 | 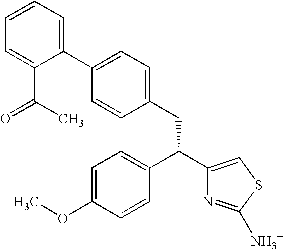 |
| 222 | 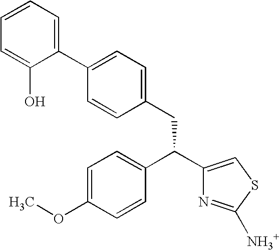 |
| 223 | 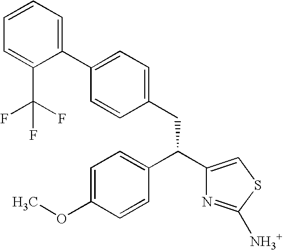 |
| 224 | 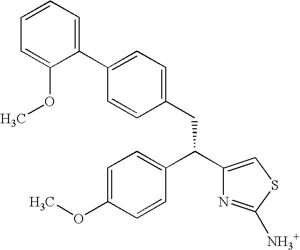 |
| 225 | 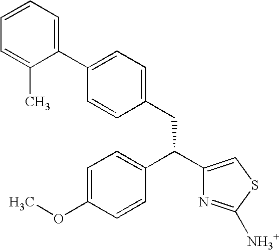 |
| 226 | 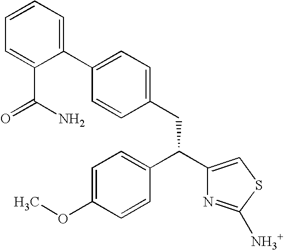 |
| 227 | 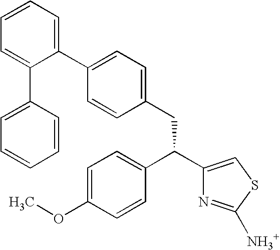 |
| 228 | 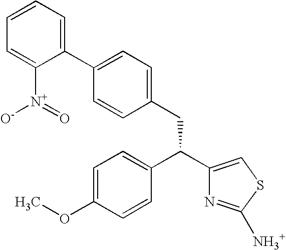 |
| 229 | 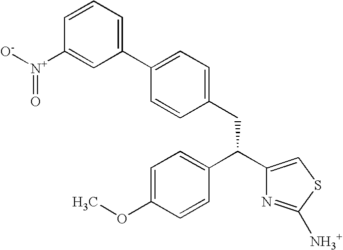 |
| 230 | 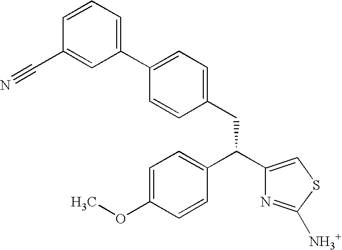 |
| 231 | 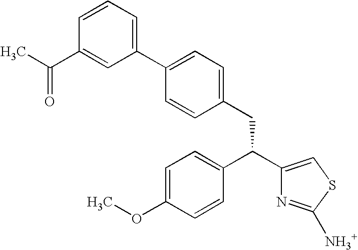 |
| 232 | 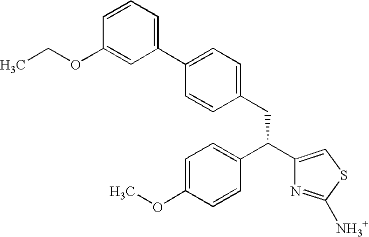 |
| 233 | 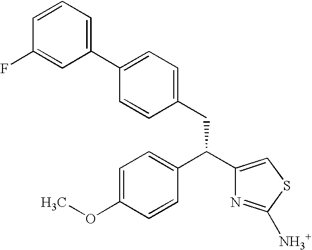 |
| 234 | 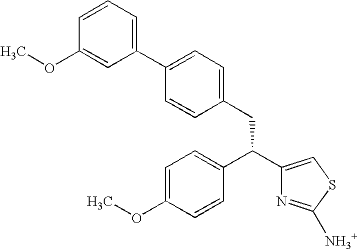 |
| 235 | 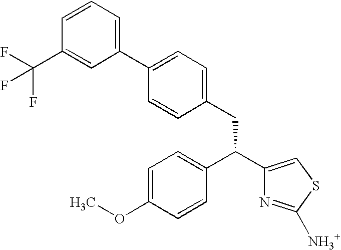 |
| 236 | 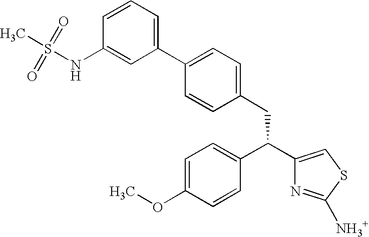 |
| 237 | 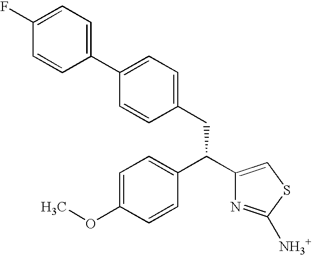 |
| 238 | 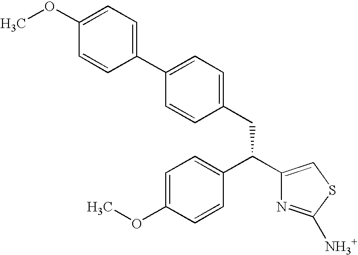 |
| 239 | 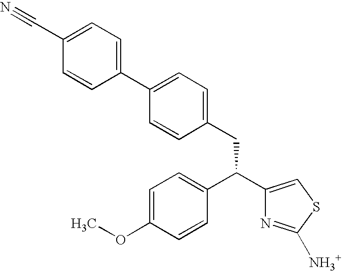 |
| 240 | 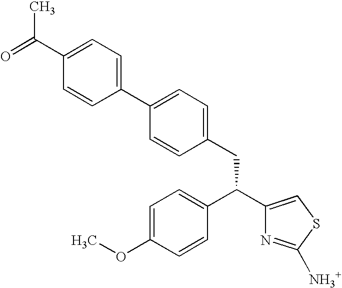 |
| 241 | 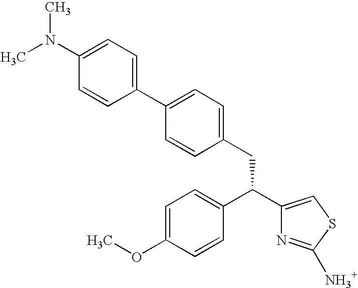 |
| 242 | 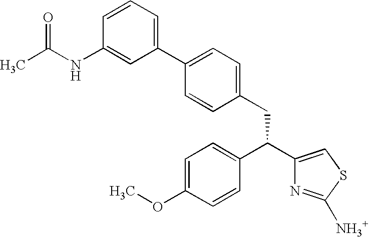 |
| 243 | 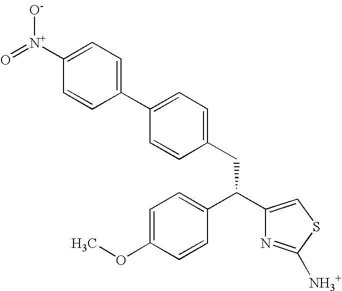 |
| 244 | 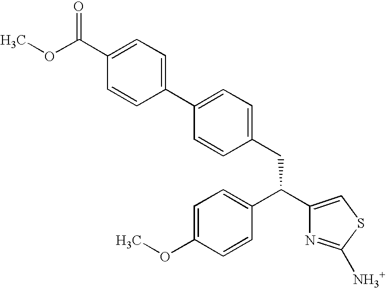 |
| 245 | 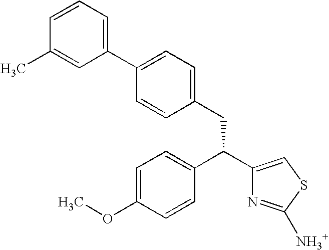 |
| 246 | 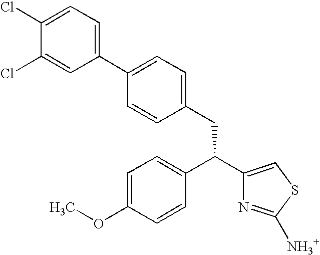 |
| 247 | 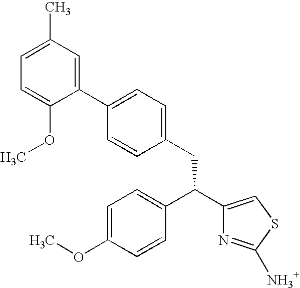 |
| 248 | 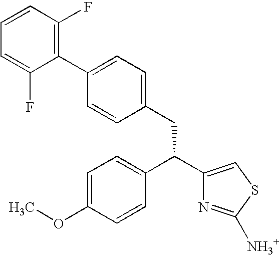 |
| 249 | 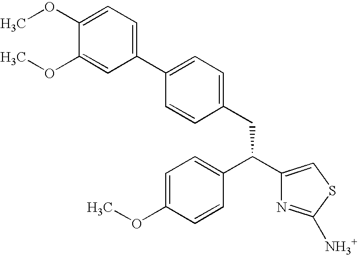 |
| 250 | 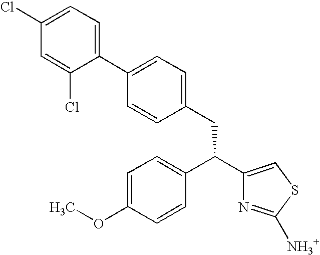 |
| 251 | 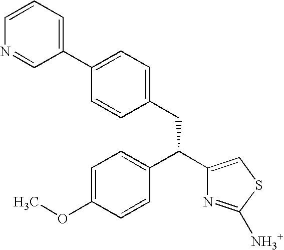 |
| 252 | 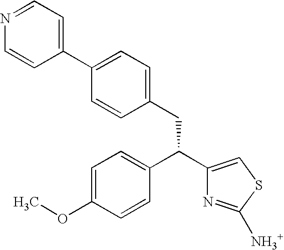 |
| 253 | 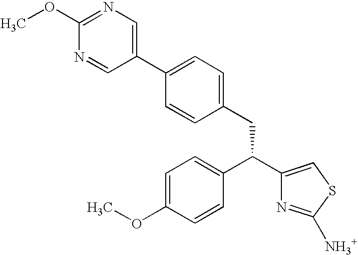 |
| 254 | 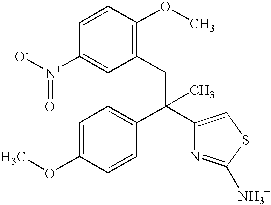 |
| 255 | 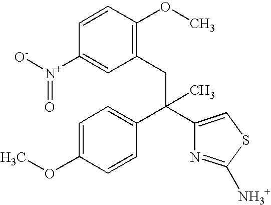 |
| 256 | 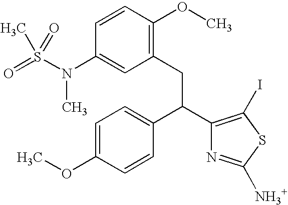 |
| 257 | 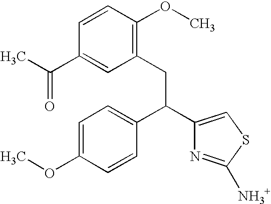 |
| 258 | 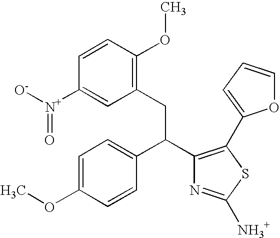 |
| 259 | 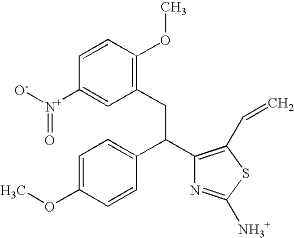 |
| 260 | 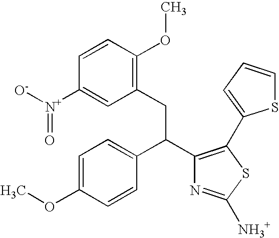 |
| 261 | 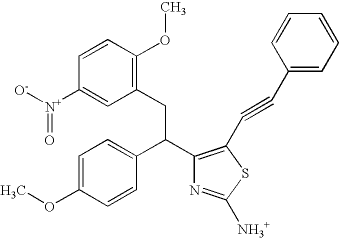 |
| 262 | 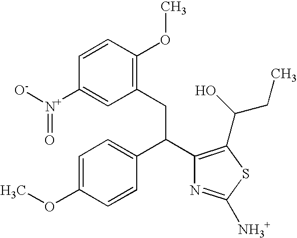 |
| 263 | 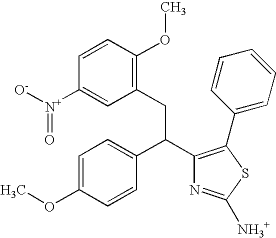 |
| 264 | 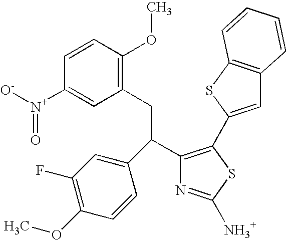 |
| 265 | 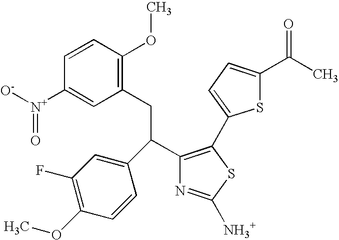 |
| 266 | 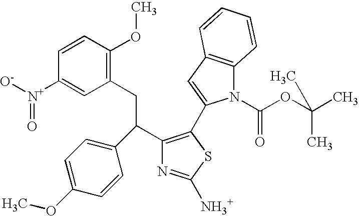 |
| 267 | 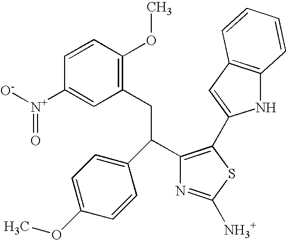 |
| 268 | 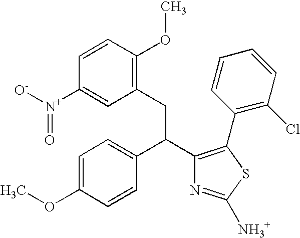 |
| 269 | 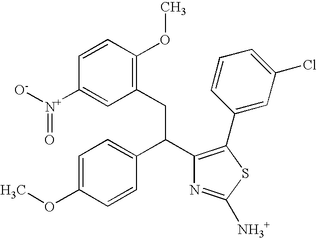 |
| 270 | 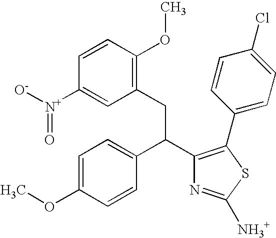 |

and pharmaceutically acceptable salts thereof.

19. The pharmaceutical composition comprising a therapeutically effective amount of a compound of claim 1 or a pharmaceutically acceptable salt thereof and a pharmaceutically acceptable carrier.

20. A method for treating Alzheimer's disease in a patient in need thereof comprising administering to the patient a therapeutically effective amount of a compound of claim 1 or a pharmaceutically acceptable salt thereof.

21. A method of inhibiting HIV protease in a subject in need thereof which comprises administering to the subject a therapeutically effective amount of a compound of claim 1 or a pharmaceutically acceptable salt thereof.

22. A method of treating infection by HIV in a subject in need thereof which comprises administering to the subject a therapeutically effective amount of a compound of claim 1 or a pharmaceutically acceptable salt thereof.

23. A method of treating AIDS in a subject in need thereof which comprises administering to the subject a therapeutically effective amount of a compound of claim 1 or a pharmaceutically acceptable salt thereof.
